(19) United States
(12) Patent Application Publication
(10) Pub. No.: US 2026/0209705 A1
(43) Pub. Date: Jul. 23, 2026

(54) ENGINEERED NATURAL KILLER CELLS AND RELATED METHODS

(71) Applicant: ASTELLAS PHARMA INC., Tokyo (JP)

(72) Inventors: Kim CLARY, Kenmore, WA (US); David RUSSELL, Seattle, WA (US); Yusuke WADA, Ibaraki (JP); Matthew HALL, Philadelphia, PA (US); Masayuki ITO, Seattle, WA (US); Eyayu BELAY, Lynwood, WA (US); Ryan DAVIS, Edmonds, WA (US); Joshua GRIMLEY, Seattle, WA (US); Tamar BOURSALIAN, Seattle, WA (US); Hyun Jung GOO, Seattle, WA (US)

(21) Appl. No.: 19/138,504

(22) PCT Filed: Dec. 28, 2023
(86) PCT No.: PCT/IB2023/063330

§ 371 Date: Jun. 12, 2025

Related U.S. Application Data
(60) Provisional application No. 63/477,785, filed on Dec. 29, 2022.

(51) Int. Cl. C12N 5/0783; A61K 35/17; A61K 38/20; A61K 39/00; A61K 39/395; A61P 35/00; C07K 14/705

(52) U.S. Cl. CPC C12N 5/0646; A61K 35/17; A61K 38/2013; A61K 38/2086; A61K 39/3955; A61P 35/00; C07K 14/70539; A61K 2039/505; C12N 2501/125; C12N 2501/2303; C12N 2501/2307; C12N 2501/2315; C12N 2501/2318; C12N 2501/2321; C12N 2501/26; C12N 2501/999; C12N 2506/45; C12N 2510/00

(57) ABSTRACT

Some embodiments relate to a population of engineered natural killer cells, wherein the NK cells express at least one polypeptide selected from the group consisting of human leukocyte antigen E, human leukocyte antigen F, and human leukocyte antigen G. The NK cells comprise a genetically engineered disruption of one or more copies of endogenous β-2 microglobulin and a genetically engineered disruption of one or more copies of a human leukocyte antigen class II-related gene selected from the group consisting of regulatory factor X associated ankyrin containing protein, regulatory factor 5, regulatory factor X associated protein, and class II transactivator. A method of producing human pluripotent stem cell-derived engineered natural killer cells under feeder free conditions also is provided, as is a method of treating a disease or disorder in a subject by administering the NK cells to the subject.

### CROSS REFERENCE TO RELATED APPLICATIONS

This application is a national phase filing under 35 C.F.R. § 371 of and claims priority to PCT Patent Application No. PCT/IB2023/063330, filed on Dec. 28, 2023, which claims the benefit of U.S. Provisional Patent Application No. 63/477,785, filed Dec. 29, 2022, the disclosure of which is hereby incorporated by reference in its entirety.

### FIELD OF THE DISCLOSURE

The presently disclosed subject matter relates to engineered natural killer (NK) cells and methods of preparing populations of engineered NK cells.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 58,824 byte file named “57819P_Seqlisting.XML”; created on Dec. 18, 2022.

### BACKGROUND

The engineering of patient-derived immune cells to express chimeric antigen receptors (CARs) has altered the landscape of adoptive cell therapies, providing scientists and clinicians the ability to harness the powerful cytolytic capabilities of immune cells and direct them to specific antigen-expressing targets in an MHC-independent manner. Natural killer (NK) cells, which arise through the lymphoid lineage, are part of the innate immune system and attractive for adoptive cell therapy as they have been found to detect and kill certain types of tumor cells. Typical sources of NK cells include peripheral blood NK cells (PB-NK), umbilical cord blood NK cells and NK cell lines, such as NK-92. While promising, these sources suffer from genetic instability, inconsistent cytotoxicity, limited expansion, product heterogeneity, and/or host immunogenicity. There is a need in the art for improved NK cell-based therapies to address limitations of current therapy options.

### SUMMARY

The disclosure provides a population of natural killer (NK) cells derived from pluripotent stem cells. The disclosure further provides a population of engineered NK cells, wherein the NK cells express at least one polypeptide selected from the group consisting of human leukocyte antigen E (HLA-E), human leukocyte antigen F (HLA-F), and human leukocyte antigen G (HLA-G). The NK cells also comprise a genetically engineered disruption of one or more copies (e.g., all copies) of endogenous β-2 microglobulin (B2M) and/or a genetically engineered disruption of one or more copies (e.g., all copies) of a human leukocyte antigen (HLA) class II-related gene selected from the group consisting of regulatory factor X associated ankyrin containing protein (RFXANK), regulatory factor 5 (RFX5), regulatory factor X associated protein (RFXAP), and class II transactivator (CIITA). For example, the NK cells comprise a genetically engineered disruption of endogenous B2M and RFXANK, and express HLA-E. In another example, the NK cells comprise a genetically engineered disruption of all copies of endogenous B2M and all copies of RFXANK, and express HLA-E. In various aspects, the NK cells further comprise a nucleic acid molecule encoding a suicide gene product (e.g., a herpes simplex virus thymidine kinase (TK) suicide gene). The NK cells are optionally derived from pluripotent stem cells (e.g., embryonic stem cells or induced pluripotent stem cells), and optionally express one or more cell surface markers selected from the group consisting of CD56, CD45, CD25, DNAM-1, NKp30, NKG2D, and NKp44. Optionally, the NK cells further express a chimeric antigen receptor (CAR); an Fc receptor (FcR); or a non-natural NKG2D receptor (see, for example, U.S. Pat. No. 10,259,858, and U.S. Patent Publication No. 2020/0138866), which, in some aspects, is capable of binding an antibody-based bispecific molecule (e.g., a MicAbody); or other type of receptor.

The disclosure further provides a method of producing pluripotent stem cell-derived NK cells under feeder free, optionally serum-free, conditions. The method comprises (a) culturing pluripotent stem cells (PSCs), optionally genetically engineered to disrupt one or more copies (e.g., all copies) of B2M and/or one or more copies (e.g., all copies) of a HLA class II-related gene selected from the group consisting of RFXANK, RFX5, RFXAP, and CIITA, and optionally express a HLA class I protein, e.g., HLA-E; (b) differentiating the pluripotent stem cells to precursor cells (e.g., using embryoid bodies) capable of differentiating to hematopoietic cell types, endothelial cell types, and/or mesodermal derivatives; and (c) differentiating the precursor cells to NK cells under feeder free, optionally serum-free, conditions in a culture media comprising one or more of SCF, IL-7, IL-15, and FIt3L. Optionally, the culture media of step (c) further comprises a pyrimido[4,5-b]indole derivative such as (1r,4r)-N1-(2-benzyl-7-(2-methyl-2H-tetrazol-5-yl)-9H-pyrimidol [4,5-b]indol-4-yl)cyclohexane-1,4-diamine dihydrobromide dihydrate (“UM171”). In various aspects, the PSC is a human PSC. In other various aspects, the method further comprises (d) culturing the NK cells in a culture media comprising IL-15 and IL-18 (and, optionally, IL-21), and in the presence of an antibody or ligand that binds a receptor on NK cells to promote activation and expansion of the NK cells. In one aspect, the antibody or ligand binds to a receptor selected from the group consisting of DNAM-1, OX40, NKG2D, 2B4, NKp30, and NKp46. A combination of antibodies or ligands which bind different targets selected from DNAM-1, OX40, NKG2D, 2B4, NKp30, and NKp46 may be included in step (d). For example, step (d) may comprise culturing the NK cells in the presence of an antibody that binds NKp30 and/or an antibody that binds DNAM-1.

The disclosure also provides a method of treating a disease in a subject in need thereof, the method comprising administering to the subject an effective amount of the population of NK cells, thereby treating the disease in the subject. In some aspects of the disclosure, the disease is cancer. Optionally, one or more cytokines, such as IL-15 and/or IL-2, are administered to the subject prior to, during, or after administering the population of NK cells. Also optionally, one or more antibody constructs are administered to the subject prior to, during, or after administering the population of NK cells.

While various embodiments in the specification are presented using “comprising” language, under various circumstances, a related embodiment may also be described using “consisting of” or “consisting essentially of” language. The disclosure contemplates embodiments described as “comprising” a feature to include embodiments which “consist of” or “consist essentially of” the feature. The term “a” or “an” refers to one or more. As such, the terms “a” (or “an”), “one or more,” and “at least one” can be used interchangeably herein. The term “or” should be understood to encompass items in the alternative or together unless context unambiguously requires otherwise.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein. However, the description also contemplates the same ranges in which the lower and/or the higher endpoint is excluded. When the term “about” is used in relation to a numerical number or general value, it means the recited number or value plus or minus 1%, 2%, 3%, 4%, 5%, 10%, or 15%, including all the values within this range, of that recited number or value. The actual variation intended is determinable from the context.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. Only such limitations which are described herein as critical to the presently disclosed subject matter should be viewed as such; variations of the presently disclosed subject matter lacking limitations which have not been described herein as critical are intended as aspects of the presently disclosed subject matter.

Additional features and variations of the presently disclosed subject matter will be apparent to those skilled in the art from the entirety of this application, including the figures and detailed description, and all such features are intended as aspects of the presently disclosed subject matter. Likewise, features of the presently disclosed subject matter described herein can be re-combined into additional embodiments that also are intended as aspects of the presently disclosed subject matter, irrespective of whether the combination of features is specified as an aspect or embodiment of the presently disclosed subject matter. The entire document is intended to be related as a unified disclosure, and it should be understood that all combinations of features described herein (even if described in separate sections) are contemplated, even if the combination of features is not found together in the same sentence, or paragraph, or section of this document.

### BRIEF DESCRIPTION OF THE DRAWINGS

FIG. 1 depicts a representative scheme for an NK differentiation process described herein: Step 0—PSC Culture; Step 1—Hematopoietic Differentiation; Step 2—NK Differentiation; Step 3A—NK Activation; and Step 3B—NK Expansion. Steps 0-3B are referenced in the figure descriptions below.

FIGS. 2A-2C are bar graphs illustrating the cell yield (FIG. 2A), percentage of CD56+CD45⁺ cells (FIG. 2B), and cell viability (FIG. 2C) resulting from the method of producing human PSC-derived engineered NK cells (FcR-UDC I-NK cells) described herein. The bar on the left of each graph represents conditions which did not include UM171, and the bar on the right in each graph represents conditions wherein 35 nM UM171 was present.

FIGS. 3A-D are bar graphs illustrating the Step 2 (NK Differentiation) yield and expansion of NK cells across multiple gene edited clones (FIGS. 3A-3B) and PSC cell lines (FIGS. 3C-D). In FIGS. 3A-3B, NK cells were derived from NIH3 PSC cell line (wild-type) or genetically engineered NIH3 PSC cell lines: UDC I-PSC, FcR-UDC I-PSC, and UDC I/II-PSC. In FIGS. 3C-3D, NK cells were derived from NIH3 and W1C1 PSC cell lines that were genetically engineered (UDC I-PSCs and UDC I/II-PSC) or parental NIH3 cell line (wild-type).

FIG. 4 is a graph illustrating the effects of various combinations of NK receptor agonists on the activation of CD56+ cells, as measured by expression of CD215/IL-15Rα (left panel) or CD25/IL-2Rα (right panel).

FIGS. 5A-5B are line graphs illustrating (FIG. 5A) cumulative fold-expansion and (FIG. 5B) cell viability after Step 3A and Step 3B of the NK differentiation process (illustrated in FIG. 1) testing different cell densities plated at the beginning of Step 3A. NK cells were generated from NIH3 UDC I-PSC using the method described herein.

FIG. 6 is a graph comparing cell sizes of NK cells derived from PSCs using the method described herein (“iPSC”) and NK cells from peripheral blood (“PBNK”) as determined by flow cytometry. NK cells derived from PSCs using the method described herein were on average about 18% larger in size (by volume) compared to NK cells isolated from peripheral blood.

FIGS. 7A-7E are bar graphs of the expected phenotype of NK cells derived from different gene edited clones: NIH3 wild-type and engineered NIH3 UDC I-PSC, FcR-UDC I-PSC, and UDC I/II-PSC. Asterisk indicates no data collected.

FIGS. 8A-8D are bar graphs of the expected phenotype of NK cells derived from different cells lines: NIH3 and W1C1 wild-type, and gene edited clones engineered UDC I-PSC and UDC I/II-PSC. Asterisk indicates no data collected.

FIGS. 9A-9C are bar graphs illustrating expression of common NK and T cell markers in FcR-UDC I-NK cells (Step 2 (left bar for each marker) and Step 3 (middle bar for each marker) derived from engineered FcR-UDC I-PSC compared to expanded PB-NK (right bar for each marker). Steps 2 and 3 are illustrated in FIG. 1.

FIGS. 10A-10C are bar graphs illustrating expression of common activation, exhaustion, and “memory-like” markers in FcR-UDC I-NK cells (Step 2 (left bar for each marker) and Step 3 (middle bar for each marker)) derived from engineered FcR-UDC I-PSC compared to expanded PB-NK (right bar for each marker). Steps 2 and 3 are illustrated in FIG. 1.

FIGS. 11A-11B are bar graphs illustrating expression of cytokine and chemokine receptors in FcR-UDC I-NK cells (Step 2 (left bar for each receptor) and Step 3 (middle bar for each receptor) derived from engineered FcR-UDC I-PSC compared to expanded PB-NK (right bar for each receptor). Steps 2 and 3 are illustrated in FIG. 1.

FIGS. 12A-12C are bar graphs illustrating expression of adhesion/homing, costimulatory, and apoptotic markers in FcR-UDC I-NK cells (Step 2 (left bar for each receptor) and Step 3 (middle bar for each receptor)) derived from engineered FcR-UDC I-PSC compared to expanded PB-NK (right bar for each receptor). Steps 2 and 3 are illustrated in FIG. 1.

FIG. 13 is a line graph illustrating natural cellular cytotoxicity (NCC) of the NK cells derived from engineered NIH3 and W1C1 PSC, UDC I-PSC (2F7 (NIH) and 1A6 (W1C1)), FcR-UDC I-PSC (3C1), and UDC I/II-PSC (10A7 (NIH) and 2A6 (W1C1)), against K562 tumor target cells. Percent specific lysis is provided on the y-axis, and effector to target cell ratio (E/T) is provided on the x-axis. The UDC-NK cells disclosed herein mediated cell lysis across multiple PSC cell lines and gene edited clones.

FIG. 14 is a line graph illustrating NCC of FcR-UDC I-NK cells compared to expanded PB-NK. Percent specific lysis is provided on the y-axis, and effector to target cell ratio (E/T) is provided on the x-axis. The UDC-NK cell population disclosed herein mediated cell lysis to a degree comparable to PB-NK.

FIG. 15 is a line graph illustrating that FcR-UDC I-NK exhibits no fratricide nor cytotoxicity against donor PBMCs, but exhibits NCC against K562 tumor target cells.

FIG. 16 is a line graph demonstrating the efficacy of engineered NK cells (FcR-UDC I-NK cell) to kill serially added K562 tumor cells in a dose-dependent manner.

FIGS. 17A-17D are line graphs illustrating antibody-dependent cellular cytotoxicity (ADCC) activity of the engineered FcR-UDC I-NK cells generated using the methods described herein. Percent specific lysis is provided on the y-axis, and effector to target cell ratio (E/T) is provided on the x-axis. In FIGS. 17A-17C, the FcR-UDC I-NK cell population disclosed herein demonstrated ADCC activity against 3 different CD20+tumor cells lines: Daudi (FIG. 17A), Ramos (FIG. 17B), and Raji (FIG. 17C) using commercially available Rituximab that binds CD20 as compared to an IgG1 control. In FIG. 17D, expanded PB-NK did not show any difference in ADCC activity between Rituximab and IgG1 control. Circles represent no antibody, squares represent human IgG1, and triangles represent Rituximab.

FIG. 18 is a bar graph illustrating NCC and ADCC activity of FcR-UDC I-NK cells across a broad range of B lymphoma cell lines.

FIG. 19A is bar graphs identifying the inducible cytokines after stimulation with Rituximab (R-mab)-coated Raji tumor cells in FcR-UDC I-NK cells (left bar for each condition noted) compared to expanded PB-NK (right bar for each condition noted). The x-axis for each graph recites “no stimulation,” “Raji+hIgG1,” and “Raji+R-mab.” Significant elevation was observed for TNF-alpha, interferon-gamma, GM-CSF, and IP-10. Moderate elevation was observed for granzyme-B, MIP-1alpha, MIP-1beta, RANTES, IL-8, IL-10, and MCP-1. Cytokines not detected included IL-1alpha, IL-1beta, IL-6, and IL-12 as shown in FIGS. 19B-19E.

FIGS. 20A-20D are bar graphs identifying cytokines and/or chemokines that were not elevated following co-culture with allogeneic PBMC versus co-culture with Raji plus Rituximab in FcR-UDC I-NK cells (left bar for each condition) compared to expanded PB-NK (right bar for each condition). The x-axis for each graph recites “no stimulation,” “Raji+Rituximab,” and “AlloPBMC.”

FIGS. 21A-21F are bar graphs illustrating in vivo persistence of FcR-UDC I-NK cells in the absence of any antigen in non-tumor-bearing mice. The NK cells (4×10⁶) were injected i.v. in the mice followed by administration of IL-2 (30,000 IU, 3×/wk.) and IL-15 (100 ng, daily for 7 days). The percent and absolute number of FcR-UDC I-NK cells in the blood, spleen, and bone marrow were measured.

FIGS. 22A-22E are bar graphs illustrating the in vivo tissue distribution of FcR-UDC I-NK cells in non-tumor-bearing mice. The NK cells (5×10⁶, 7.5×10⁶, and 10×10⁶) were injected i.v. followed by administration of IL-2 (30,000 IU, 3×/wk.) and IL-15 (100 ng, daily). The percentage of human CD56+ cells (FcR-UDC I-NK cells) within mouse plus human CD45 was measured in the lung (FIG. 22B), liver (FIG. 22D), spleen (FIG. 22E), blood (FIG. 22A), and bone marrow (FIG. 22C).

FIG. 23 is a graphic of an efficacy study of FcR-UDC I-NK cells plus Rituximab in a Raji i.p. tumor model. NSG mice were irradiated and injected (i.p.) with Raji tumor cells (3×10⁵). At day 4, FcR-UDC I-NK cells or PB-NK cells (each 5×10⁶) with or without Rituximab (300 μg) was administered. At day 7, an additional dose of Rituximab (300 μg) was administered. Imaging/monitoring of the NSG mice occur at indicated time points through day 120.

FIGS. 24A-24C illustrate (FIG. 24A) a survival curve and (FIGS. 24B-24C) tumor burden following the efficacy study of FIG. 23. In FIG. 24A, both FcR-UDC I-NK cells plus Rituximab and PB-NK plus Rituximab had a significant increase in median survival (days) as compared to untreated, Rituximab, FcR-UDC I-NK cells, and PB-NK alone. Rituximab and PB-NK alone increased mean survival (days) compared to untreated. In FIGS. 24B-24C, FcR-UDC I-NK cells plus Rituximab and PB-NK plus Rituximab provided a durable response in delaying tumor onset through day 60 in 50% of the mice.

FIG. 25 is a graphic of an efficacy study of FcR-UDC I-NK cells plus Rituximab in a Raji i.p. tumor model. NSG mice were irradiated and injected (i.p.) with Raji tumor cells (2×10⁵). At day 2, FcR-UDC I-NK cells (10×10⁶) with or without Rituximab (100 μg) was administered. At days 6, 10, and/or 16, additional doses of FcR-UDC I-NK cells or PB-NK cells (each 10×10⁶) were administered. Imaging/monitoring of the NSG mice occur at indicated time points through day 86.

FIGS. 26A-26B illustrate (FIG. 26A) tumor burden and (FIG. 26B) a survival curve following the efficacy study of FIG. 25. In FIG. 26A, FcR-UDC I-NK cells plus Rituximab (D2, 10, and 16) had longer durable response in delaying tumor onset compared to FcR-UDC I-NK cells plus Rituximab (D2, 6, and 10). In FIG. 26B, both FcR-UDC I-NK cells plus Rituximab (regardless of dosing regimen) had a significant increase in median survival (days) as compared to untreated, Rituximab, and FcR-UDC I-NK cells alone.

FIGS. 27A-27B are related to an efficacy study using NOGf mice that have mouse FcgR knocked out. FIG. 27A is a graphic of the study wherein Rituximab or FcR-UDC I-NK cells plus Rituximab were administered in a Raji tumor model. NOGf mice were injected (i.v.) with Raji tumor cells (1×10⁵). Rituximab (100 μg, i.p.) was administered on days 1, 8, and 15 following tumor cell implantation, and FcR-UDC-I-NK (10M) was administered i.v. on days 1, 4, 8, and 15. FIG. 27B illustrates tumor burden following the efficacy study of FIG. 27A. Cryopreserved FcR-UDC I-NK cells plus Rituximab demonstrated a greater degree of tumor growth inhibition compared to administration of Rituximab alone.

FIGS. 28A-28B are related to an efficacy study using NOGf mice engineered to express human interleukin 15 (IL-15) and that have mouse FogR knocked out. FIG. 28A is a graphic of the study wherein Rituximab (50 or 100 μg, i.p.) or FcR-UDC I-NK cells (3×10⁶ (3M) or 15×10⁶ (15M), i.v.) plus Rituximab (50 or 100 μg, i.p.) were administered in a Raji tumor model. FIG. 28B illustrates tumor burden following the efficacy study of FIG. 28A. 15M cryopreserved FcR-UDC I-NK cells plus Rituximab demonstrated a great degree of tumor growth inhibition compared to Rituximab alone.

FIG. 29 is a line graph illustrating the anti-tumor effect of FcR-UDC I-NK cells plus anti-claudin 6 (CLDN6) antibodies (ASP1893 mAb, a humanized anti-CLDN6 antibody and ASP1650, a human/mouse chimeric anti-CLDN6 antibody) against CLDN6-positive tumor cells (the ovarian cancer cell line PA-1). The specific lysis (%) is presented on the y-axis and the amount of antibody ASP1893 mAb or ASP1650 (ng/ml) is presented on the x-axis. Anti-CLDN6 antibodies ASP1893 mAb and ASP1650 induced comparable ADCC with FcR-UDC I-NK cells against CLDN6+ tumor cells.

FIGS. 30A-30M are line graphs illustrating the anti-tumor effect of FcR-UDC I-NK cells plus anti-CLDN6 antibodies against tumor cells expressing various levels of CLDN6. Assays were conducted with FcR-UDC I-NK cells with antibody ASP1893 mAb with control IgG1 antibody, or with no antibody. FIGS. 30A-30D represent assays using tumor cells which highly express CLDN6 (SKOV3-CLDN6, ES-2-CLDN6, PA-1, and NEC14 cells). FIGS. 30E-30G represent assays using tumor cells which express moderate levels of CLDN6 (ABC-1, OV90, and OVCAR3 cells). FIGS. 30H-30I represent assays using tumor cells which express low levels of CLDN6 (COV362 and NEC8-Luc cells). FIGS. 30J-30M represent assays using tumor cells which express no CLDN6 (COV318, ES-2, SKOV3, and NCI-H1373 cells). The specific lysis (%) is presented on the y-axis and the E/T ratio is presented on the x-axis. FcR-UDC I-NK cells mediated ADCC with ASP1893 mAb against tumor cells in CLDN6 expression dependent manner.

FIGS. 31A-31B illustrate cytokine secretion of FcR-UDC I-NK cells. The figures are bar graphs correlating interferon-gamma (IFN-g; pg/mL) or tumor necrosis factor-alpha (TNF-a) produced by FcR-UDC I-NK cells in the presence of tumor cells (left bar for each cell line tested), tumor cells with human IgG1 (middle bar for each cell line tested), and tumor cells with anti-CLDN6 antibody (ASP1893 mAb) (right bar for each cell line tested). SKOV3-CLDN6, ES-2-CLDN6, PA-1, and NEC14 cells display high levels of CLDN6. ABC-1 and OV90, cells display moderate levels of CLDN6. COV362 and NEC8-Luc cells express low levels of CLDN6. COV318, ES-2, and SKOV3 cells are CLDN6-negative. FcR-UDC I-NK cells exhibited IFN-g and TNF-α production upon stimulation with tumor cells and ASP1893 mAb in a CLDN6 expression-dependent manner.

FIG. 32 is a schematic of an in vivo assay examining the anti-tumor efficacy of UDC-NK cells in conjunction with anti-CLDN6 antibody in a PA-1 xenograft solid tumor mouse model. The NOGf IL-15 mice expressed human IL-15 and lacked mouse FcgR.

FIGS. 33A-33B illustrate tumor volume (FIG. 33A) and survival curve (FIG. 33B) following the efficacy study of FIG. 32. The combination treatment of FR-UDC-NK cells and anti-CLDN6 antibody resulted in tumor growth inhibition (FIG. 33A) and prolonged the survival of mice in PA-1 SC tumor model (FIG. 33B).

FIGS. 34A-34C are related to a study examining the anti-tumor efficacy of UDC-NK cells in conjunction with anti-CLDN6 antibody in a PA-1 xenograft solid tumor mouse model using NOGf mice engineered to express human interleukin 15 (IL-15) and that have mouse FcgR knocked out. FIG. 34A is a schematic of the in vivo assay wherein 7.5M FcR-UDC-NK cells were administered i.v. on days 16, 20, 23, and 27 and anti-CLDN6 antibody (300 μg, i.p.) was administered on days 16 and 23. FIGS. 34B-34C illustrate tumor volume (FIG. 34B) and survival curve (FIG. 34C) following the efficacy study of FIG. 34A. The combination treatment of FcR-UDC-NK cells and anti-CLDN6 antibody resulted in tumor growth inhibition (FIG. 34B) and prolonged the survival of mice in PA-1 SC tumor model (FIG. 34C).

FIGS. 35A-35B illustrate ADCC activity of FcR-UDC-NK cells. FIG. 35A is a line graph illustrating the anti-tumor effect of FcR-UDC I-NK cells plus anti-claudin 18.2 antibody (Zolbetuximab) against BxPC3-CLDN18.2-Luc tumor cells. The specific lysis (%) is presented on the y-axis and the E:T ratio is presented on the x-axis. FIG. 35B is a line graph illustrating the anti-tumor effect of FcR-UDC I/II-NK cells plus anti-claudin 18.2 antibody (Zolbetuximab) against BxPC3-CLDN18.2-Luciferase tumor target cells. The fluorescent intensity of the remaining target cells, normalized to the start of the experiment, is presented on the y-axis and the time of measurement is presented on the x-axis.

FIGS. 36A-36B are bar graphs illustrating interferon-gamma (FIG. 36A) or tumor necrosis factor-alpha (FIG. 36B) secretion by FcR-UDC I-NK in samples comprising BxPC3-CLDN18.2-Luc tumor cells alone, BxPC3-CLDN18.2-Luc tumor cells with human IgG, or BxPC3-CLDN18.2-Luc tumor cells with Zolbetuximab.

### DETAILED DESCRIPTION

The instant disclosure provides a population of natural killers (NK) cells with advantageous attributes for immune cell therapy. The materials and methods described herein enable production of, e.g., a substantially homogenous population of NK cells with reduced occurrence of undesired cells, improved safety profiles, strong persistence in vivo, enhanced ability to engraft in a subject, and/or enhanced ability to reduce tumor burden.

The disclosure provides a population of NK cells derived from pluripotent stem cells, including embryonic stem cells (ESC) and induced pluripotent stem cells (iPSC). The disclosure further provides a population of engineered NK cells, wherein the NK cells express at least one exogenous polypeptide selected from the group consisting of human leukocyte antigen E (HLA-E), human leukocyte antigen F (HLA-F), and human leukocyte antigen G (HLA-G). The NK cells are genetically engineered to disrupt of one or more copies (e.g., all copies) of the cells' endogenous β-2 microglobulin (B2M) and/or one or more copies (e.g., all copies) of a human leukocyte antigen (HLA) class II-related gene selected from the group consisting of regulatory factor X associated ankyrin containing protein (RFXANK), regulatory factor 5 (RFX5), regulatory factor X associated protein (RFXAP), and class II transactivator (CIITA). The disruptions allow the NK cells to, e.g., reduce or evade immune detection, and allow production of an NK cell population which does not require HLA matching for therapeutic applications. Optionally, the NK cells also express a chimeric antigen receptor (CAR) (e.g., a universal CAR), an Fc receptor (FcR or CD16), a non-natural NKG2D receptor, or other type of receptor.

NK cells are cytotoxic lymphocytes of the innate immune system capable of immune surveillance. NK cells are attractive therapeutics for their natural cytotoxicity to foreign, transformed, or virally infected cells, their antibody-dependent cellular cytotoxicity, and cytokine release profile which can amplify an immune response. NK cells are typically defined by the expression of CD56 or CD16 and the absence of CD3. In various aspects of the disclosure, the NK cells express one or more (e.g., two or more, three or more, four or more, etc.) cell surface markers selected from CD56, CD45, CD25, DNAM-1, NKp30, NKG2D, and/or NKp44. Methods of detecting cell surface markers are known in the art and include, for instance, flow cytometry-based methods.

The NK cells (or a PSC from which the NK cell is derived or an intermediate thereof) are genetically engineered to disrupt of one or more copies (e.g., all copies) of the cells' endogenous B2M gene. B2M is a critical component of the HLA class I (HLA-I) complex. HLA is a cell surface complex that mediates leukocyte-leukocyte interactions or interactions of leukocytes with other cells. HLA-I molecules are cell surface complexes which present antigens to CD8+ cytotoxic T cells, thereby mediating cellular immunity. HLA-I molecules comprise an HLA-I heavy chain and B2M. Genetic disruption of one or more (e.g., all) copies of the B2M gene reduces or abrogates production of the B2M protein (SEQ ID NO: 5), thereby reducing or abrogating display of HLA-I complexes on the NK cell. A representative nucleic acid sequence encoding B2M is provided in SEQ ID NO: 1.

Also contemplated are NK cells (or a PSC from which the NK cell is derived or an intermediate thereof) genetically engineered to disrupt one or more copies (e.g., all copies) of a subset of HLA-I. Six HLA class I alpha (α) chains have been identified, including three classical (HLA-A, HLA-B and HLA-C) and three non-classical (HLA-E, HLA-F, and HLA-G), which are responsible for specificity of peptide binding on the HLA-I binding cleft. In this regard, the disclosure contemplates NK cells genetically engineered to disrupt one or more copies (e.g., all copies) of an HLA-I gene selected from HLA-A, HLA-B, HLA-C, HLA-E, HLA-F, and HLA-G, as well as combinations thereof. For example, the NK cells (or a PSC from which the NK cell is derived or an intermediate thereof) may be genetically engineered to disrupt one or more copies (e.g., all copies) of HLA-A, HLA-B, and/or HLA-C. HLA molecules are further described in, e.g., Choo, Yonsei Med J., 48 (1): 11-23 (2007); www.ebi.ac.uk/ipd/imgt/hla/; and GenBank Accession Nos. NG_029217 (HLA-A), XP_041536701 (HLA-A), NG_023187 (HLA-B), NP_005505 (HLA-B), NG_029422 (HLA-C), NP_001229971 (HLA-C), NM_005516 (HLA-E), NP_005507 (HLA-E), NG_012009 (HLA-F), NP_001091949 (HLA-F), NG_029039 (HLA-G), and NP_001371219 (HLA-G).

The NK cells of the disclosure (or a PSC from which the NK cell is derived or an intermediate thereof) are genetically engineered to disrupt one or more (e.g., all) copies of an HLA class II (HLA-II)-related gene selected from RFXANK, RFX5, RFXAP, and CIITA. HLA-II molecules are transmembrane proteins found on antigen-presenting cells (APCs) and, at times, solid organs. HLA-II molecules comprise two homologous subunits, the alpha (α) subunit and the beta (β) subunit. HLA-II related genes encode HLA-II regulatory proteins that regulate the expression of HLA class II molecules. Genetic disruption of one or more copies (e.g., all copies) of one or more of the HLA-II related genes RFXANK, RFX5, RFXAP, and/or CIITA reduces or abrogates production of the encoded proteins, thereby reducing or abrogating display of HLA-II complexes on the NK cell. The NK cell (or a PSC from which the NK cell is derived or an intermediate thereof) may comprise disruptions of one or more copies (e.g., all copies) of the genes encoding one of RFXANK, RFX5, RFXAP, and/or CIITA (e.g., the cell is engineered to disrupt one or more copies (e.g., all copies) of the RFXANK gene); one or more copies (e.g., all copies) of the genes encoding any combination of two of RFXANK, RFX5, RFXAP, and/or CIITA; one or more copies (e.g., all copies) of the genes encoding any combination of three of RFXANK, RFX5, RFXAP, and/or CIITA; or one or more copies (e.g., all copies) of the genes encoding all of RFXANK, RFX5, RFXAP, and CIITA. RFXANK is encoded by, e.g., the sequence of SEQ ID NOs: 3 and 5; RFX5 is encoded by, e.g., the sequence of SEQ ID NOs: 7 and 9; RFXAP is encoded by, e.g., SEQ ID NO: 11; and CIITA is encoded by, e.g., SEQ ID NO: 13. In various aspects of the disclosure, the NK cells are engineered to disrupt all copies of B2M and RFXANK.

Any suitable technique for introducing a disruption in a target gene (e.g., an HLA-II related gene, a B2M gene, or any other gene of interest) may be used. Many techniques for disrupting endogenous coding sequences are known in the art, including use of gene editing systems such as CRISPR/Cas (clustered regularly interspaced short palindromic repeats and CRISPR-associated protein) systems, transcription activator-like effector nucleases (TALENs), and zinc finger nucleases, as well as targeting vectors that cleave and/or insert nucleic acid sequences into a target site in the cellular genome. An exemplary technique employs adeno-associated virus (AAV) vector-based editing, which optionally involves targeted insertion of an edited sequence (potentially a transgene) into the cellular genome so as to interrupt an endogenous coding sequence, thereby resulting in reduction or avoidance of production of the endogenous protein. In various aspects, serotype 3B AAV vectors are employed. Use of AAV vectors to engineer stem cells is further described in, e.g., Riolobos et al., Molecular Therapy, 21 (6), 1232-1241 (2013), hereby incorporated by reference in its entirety and particularly with respect to the disclosure of use of AAV vectors and disruption of HLA complex production. Methods of confirming disruption of a target gene are known in the art and include, but are not limited to, PCR detection methods (e.g., quantitative real time polymerase chain reaction (qRT-PCR)), RNA sequencing, next generation sequencing methods, flow cytometry, and immunostaining.

In various aspects, the engineered NK cells express at least one polypeptide selected from the group consisting of HLA-E, HLA-F, and HLA-G. In the context of the disclosure, the NK cell is engineered to express HLA-E, HLA-F, and/or HLA-G, i.e., a nucleic acid encoding HLA-E, HLA-F, and/or HLA-G is introduced into the NK cell (or a PSC from which the NK cell is derived or an intermediate thereof) and the encoded protein is produced and displayed on the cell surface. Optionally, the engineered NK cells express HLA-E. Optionally, the engineered NK cell expresses a single chain fusion HLA-E comprising at least of portion of B2M linked, either directly or via a linker sequence, to at least of portion of the HLA-I α-chain, e.g., HLA-E. Optionally, the engineered NK cell expresses a single chain fusion HLA-F or HLA-G comprising at least of portion of B2M linked, either directly or via a linker sequence, to at least of portion of the HLA-I alpha-chain, e.g., HLA-F or HLA-G. The engineered NK cell may overexpress a protein which is naturally produced by the cell, may express a protein encoded by an exogenous nucleic acid following knock-out or disruption of the corresponding endogenous coding sequence for the protein, or may express a protein which is not naturally encoded by the wild-type NK cell genome. Representative DNA and protein sequences for HLA-E are provided in SEQ ID NOs: 15 and 16. Representative DNA and protein sequences for HLA-F are provided in SEQ ID NOs: 17 and 18. Representative DNA and protein sequences for HLA-G are provided in SEQ ID NOs: 19 and 20. It will be appreciated that variants of the sequences provided herein may be used and, as such, the disclosure contemplates use of a nucleic acid comprising at least 75%, 80%, 85%, 90%, 91%, 92%, 93%, 94%, 95%, 96%, 97%, 98%, or 99% identity to the nucleic acid sequences disclosed herein. The disclosure further contemplates use of a nucleic acid which encodes a peptide comprising an amino acid sequence having at least 75%, 80%, 85%, 90%, 91%, 92%, 93%, 94%, 95%, 96%, 97%, 98%, or 99% identity to any of the amino acid sequences described herein. Preferably, a variant retains HLA-I functions such as, e.g., forming a functional peptide binding cleft to display peptides and/or engaging inhibitory receptors on NK cells. Cells which express HLA-E, HLA-F, and/or HLA-G minimize the risk of rejection when used as an adoptive cell therapy. In various aspects of the disclosure, the NK cells are engineered to disrupt all copies of B2M and RFXANK and engineered to express HLA-E, e.g., single chain fusion HLA-E.

The NK cells of the population also may express an exogenous receptor (i.e., a receptor encoded by an exogenous nucleic acid introduced into the NK cells or a PSC from which the NK cell is derived or an intermediate thereof). In various aspects, the NK cells of the population express a chimeric antigen receptor. “Chimeric antigen receptor” or “CAR” refers to an artificial immune cell receptor that is engineered to recognize and bind to an antigen expressed by a target cell, such as a tumor cell. Generally, a CAR is a chimera of a signaling domain of the T cell receptor (TCR) complex and an antigen-recognizing domain (e.g., a single chain fragment (scFv) of an antibody or other antibody fragment). See, e.g., Enblad et al., Human Gene Therapy. 2015; 26 (8): 498-505. There are various formats of CARs, each of which contains different components. “First generation” CARs join an antigen binding domain to the CD3zeta intracellular signaling domain of the T-cell receptor through hinge and transmembrane domains. A cytoplasmic or intracellular signaling domain produces stimulatory signals for proliferation and effector function when the CAR engages with a target antigen. Many intracellular signaling domains contain signaling motifs which are known as immunoreceptor tyrosine-based activation motifs (ITAMs). Examples of ITAM containing cytoplasmic signaling sequences include those derived from CD8, CD3zeta, CD3gamma, CD3epsilon, CD32 (Fc gamma Rlla), DAP10, DAP12, CD79a, CD79b, FcγRly, FcγRIIIγ, FcεRIβ (FCERIB), and FcεRIγ (FCERIG). Intracellular signaling domains, including those derived from CD3 zeta (CD3), are known in the art (TCR zeta, e.g., GenBank Accession No. BAG36664.1). Exemplary hinge domains include, but are not limited to, a CD28 hinge, a CD8 alpha hinge, a human IgG4 hinge domain, and a human IgG4 hinge domain combined with a CH3 human IgG4 domain. Hinge domains are further described in, e.g., Hudecek et al. (2013) Clin. Cancer Res., 19:3153, International Patent Publication No WO 2014031687, and U.S. Pat. No. 8,822,647. “Second generation” CARs incorporate an additional domain, e.g., CD28, 4-1BB (41BB), or ICOS, to supply a costimulatory signal. Additional costimulatory domains include, but are not limited to, CD27, CD134, OX40, CD149, DAP10, CD30, IL2-R, IL7r6, IL21-R, NKp30, NKp44, CD40, CD137, PD-1, ICOS, lymphocyte function-associated antigen-1 (LFA-1), CD2, CD7, LIGHT, NKG2C, B7-H3, or DNAM-1 costimulatory domains (or any combination thereof). “Third generation” CARs contain two costimulatory domains fused with the TCR CD3zeta chain. Third generation costimulatory domains may include, e.g., a combination of CD3zeta, CD27, CD28, 4-1BB, ICOS, or OX40. CARs so constructed can trigger, e.g., cell activation upon binding the targeted antigen in a manner similar to an endogenous T cell receptor, but independent of the major histocompatibility complex (MHC).

Optionally, the NK cells may express a fragment crystallizable (Fc) region receptor (FcR), e.g., CD16 (FcγRIII). In this regard, the NK cell may be engineered to express an FcR, i.e., a nucleic acid encoding an FcR is introduced into the NK cell (or a PSC from which the NK cell is derived or an intermediate thereof) and the encoded protein is produced and displayed on the cell surface. Generally, an FcR comprises an extracellular domain, a transmembrane domain, and an intracellular domain. The extracellular domain generally includes a domain which binds to an immunoglobulin (lg) Fc region. The intracellular domain comprises a sufficient portion of an Fc receptor to allow the ITAM to initiate cell signaling when an Ig Fc region binds to the extracellular domain. Cells expressing FcR mediate the efficacy of both antibody-based therapeutics and, to a lesser extent, B-cell mediated adaptive immune responses. NK cells are mediators of antibody-dependent cell-mediated cytotoxicity (ADCC) driven by Fc binding to FcRs. In ADCC, an NK cell FcR binds an antibody and forms a lytic synapse at the interface with the target cell, triggering the release of cytokines and lytic granules. These granules contain enzymes like perforin and granzyme B, which induce the destruction of the antibody-coated target cell. Engineered NK cells expressing an Fc region receptor are referenced herein as “FcR-UDC-NK cells” or “FcR-NK cells.”

The NK cell may express an Fc-gamma (γ) receptor, an Fc-alpha (α) receptor, or an Fc-epsilon (ε) receptor. The Fcγ family of receptors bind IgG and include FcγRI (CD64), FcγRII (CD32, including CD32a, CD32b, and CD32c), and FcγRIII (CD16, including CD16a and CD16b). CD 16a has two allelic variants, 158V and 158F, that possess different affinities for IgG; the NK cell of the disclosure may express CD16a, CD16b, or both. Cancer patients that are homozygous for the 158V higher affinity allele of CD16a reportedly respond better to monoclonal antibody (mAb) therapeutics (see, e.g., Cartron et al. Blood. 99, 754-58 (2002). FcRn also binds lgG. The Fcε family of receptors bind IgE and include FcεRI and FcεRII (CD23). The Fcα family of receptors bind IgA and include FcαRI (CD89) and Fcα/μR. The NK cells may express any one or more of these FcRs. Additionally, the FcR may be a chimeric FcR, wherein part of the FcR structure is derived from one type of FcR and another part is derived from a different FcR (or is synthetic by which functions in a similar fashion to an FcR subunit).

The NK cell may express a non-natural NKG2D receptor, such as a receptor comprising non-natural NKG2D receptor ectodomain that binds modified NKG2D ligand α1-α2 domains with greater affinity than it does natural α1-α2 domains. Non-natural NKG2D receptors are further described in, for example, U.S. Pat. No. 10,259,858, and U.S. Patent Publication No. 2020/0138866, each of which are incorporated by reference in their entireties and particularly with respect to their disclosure of modified NKG2D ectodomains.

The NK cells of the population also may express an exogenous polypeptide (i.e., a polypeptide encoded by an exogenous nucleic acid introduced into the NK cells or a PSC from which the NK cell is derived or an intermediate thereof). In various aspects, the NK cells of the population express a cytokine, chemokine, or the like, including, but not limited to, IL-15 or IL-2

Optionally, the NK cells further comprise a nucleic acid molecule encoding a suicide gene product. Combinations of nucleic acid molecules encoding different suicide gene products also are contemplated. Suicide genes are useful in selectively killing cells by inducing apoptosis or converting a nontoxic compound to a toxic compound, resulting in death of unwanted cells. Examples of suicide gene products include, but are not limited to, caspases (e.g., caspase 9), thymidine kinases, cytosine deaminases, cytochrome P450, and DNases. See, e.g., U.S. Patent Publication No. 2022/0025001, hereby incorporated by reference. Optionally, the nucleic acid encoding the suicide gene product is operably linked to an inducible promoter. See, e.g., Straathof et al. Blood, 10⁵ (11), 4247-4254 (2005), hereby incorporated by reference. Optionally, the engineered NK cells comprise a herpes simplex virus thymidine kinase (TK) suicide gene. See, e.g., Bonini, et al., Science, 276, 1719-1724 (1997), hereby incorporated by reference. Optionally, the engineered NK cells comprise one or more copies of the TK suicide gene. In various aspects, the NK cell comprises a nucleic acid encoding a hygromycin-thymidine kinase fusion protein. For example, NK cells may comprise a nucleic acid encoding HSV TK and a nucleic acid encoding a hygromycin-thymidine kinase fusion protein. In another embodiment, the NK cells may be eliminated by targeted antibody-mediated depletion using any number of NK cell-specific targets, including, but not limited to, CD56, CD45, CD25, DNAM-1, NKp30, NKG2D, and/or NKp44.

Methods of genetically engineering a host cell to stably produce one or more gene products of interest are known in the art. Exemplary methods involve the use of viral vectors. Viral vectors may include any suitable viral vectors including, for example, retrovirus, adenovirus, parvovirus (for example, adeno-associated viruses), coronavirus, ortho-myxovirus (for example, influenza virus), rhabdovirus (for example, rabies and vesicular stomatitis virus), paramyxovirus (for example, measles and Sendai), picornavirus, alphavirus, herpesvirus (for example, Herpes Simplex virus types 1 and 2, Epstein-Barr virus, cytomegalovirus), and poxvirus (for example, vaccinia, fowlpox, and canarypox). Exogenous nucleic acids also may be introduced into a host cell via gene editing techniques using, e.g., the systems described herein. Preferably, the nucleic acid encoding the CAR or FcR and the nucleic acid encoding HLA-E, HLA-F, or HLA-G are integrated into cellular genome, such as into the B2M or the HLA-II loci. Thus, in various aspects, the B2M loci are disrupted by inserting in the B2M loci a nucleic acid encoding a different gene product to replace the expression of the endogenous wild type B2M protein. Alternatively or in addition, certain HLA-II loci are optionally disrupted by inserting in the HLA-II loci a nucleic acid encoding a different gene product to replace the expression of the endogenous wild type HLA-II protein.

In various aspects of the disclosure, the NK cells are derived from pluripotent stem cells. The term “pluripotent stem cells” includes embryonic stem cells, embryo-derived stem cells, and induced pluripotent stem cells, regardless of the method by which the pluripotent stem cells are derived. Pluripotent stem cells are typically defined functionally as stem cells that are: (a) capable of inducing teratomas when transplanted in immunodeficient (SCID) mice; (b) capable of differentiating to cell types of all three germ layers (e.g., can differentiate to ectodermal, mesodermal, and endodermal cell types); and (c) express one or more markers of embryonic stem cells (e.g., express October 4, alkaline phosphatase, SSEA-3 surface antigen, SSEA-4 surface antigen, Nanog, TRA-1-60, TRA-1-81, SOX2, REX1, etc.). Pluripotent stem cells can be generated using, for example, methods known in the art. Exemplary pluripotent stem cells include embryonic stem cells. “Embryonic stem cells” (ESC) include, e.g., cells derived from the inner cell mass of human blastocysts or morulae, including those that have been serially passaged as cell lines. Embryonic stem cells, regardless of their source or the particular method used to produce them, can be identified based on, for instance, (i) the ability to differentiate into cells of all three germ layers, (ii) expression of at least Oct-4 and alkaline phosphatase, and (iii) ability to produce teratomas when transplanted into immunodeficient animals.

Another exemplary pluripotent stem cell is an induced pluripotent stem cell (iPSC). iPSCs are generated by, e.g., reprogramming a somatic cell by expressing a combination of factors (“reprogramming factors”) such as, but not limited to, Oct4, Sox2, c-Myc, Nanog, Lin28, and/or Klf4. Induced pluripotent stem cells can be generated using, as a starting point, virtually any somatic cell of any developmental stage. For example, the cell can be from an embryo, fetus, neonate, juvenile, or adult donor. Exemplary somatic cells that can be used include CD34+ cord blood cells, fibroblasts, such as dermal fibroblasts obtained by a skin sample or biopsy, synoviocytes from synovial tissue, foreskin cells, cheek cells, or lung fibroblasts. In certain embodiments, the somatic cell is not a fibroblast.

The pluripotent stem cells can be from any species. Embryonic stem cells have been successfully derived from, for example, mice, multiple species of non-human primates, and humans. Thus, one of skill in the art can generate embryonic stem cells from any species, including but not limited to, human, non-human primates, rodents (mice and rats), ungulates (cows, sheep, etc.), dogs (domestic and wild dogs), cats (domestic and wild cats such as lions, tigers, and cheetahs), rabbits, hamsters, gerbils, squirrel, guinea pig, goats, elephants, panda (including giant panda), pigs, raccoon, horse, zebra, marine mammals (dolphin, whales, etc.) and the like. Similarly, iPSCs can be from any species. iPSCs have been successfully generated using, for instance, mouse and human cells. Accordingly, one can readily generate an iPSC using a donor cell from any species, such as any species described herein.

The population of NK cells of the disclosure has a variety of attributes making the population advantageous for therapeutic applications, e.g., superior engraftment, homing, viability, persistence, immune avoidance, and/or cytotoxicity. For example, in various aspects, the population of engineered NK cells is capable of engrafting into a subject following administration, using, for example, protocols known to one of skill in the art. Put another way, the population of NK cells is capable of homing to a target tissue (e.g., bone marrow) and/or persisting in a subject for a period after administration (e.g., from 24 hours to a few days or weeks or months). In various aspects, at least a portion of the population of engineered NK cells (e.g., at least 1%, at least 2%, at least 5%, at least 10%, at least 20%, at least 30%, at least 40% or the population) is capable of engrafting into a target tissue. For example, in various aspects, at least 50% (e.g., at last 55%, at least 60%, at least 65%, at least 70%, at last 75%, at least 80%, at least 85%, at least 90%, at least 95%, or 100%) of the population of engineered NK cells is capable of engrafting into a target tissue (e.g., blood or bone marrow) of the subject. Optionally, the population of engineered NK cells, once administered to a subject, is capable of remaining engrafted in the target tissue for at least 1 day (e.g., at least 2 days, at least 5 days, at least 10 days). In this regard, at least a portion of the population of NK cells is capable of remaining engrafted in the target tissue for at least 20 days (e.g., at least 50 days, at least 100 days, at least 200 days, at least 500 days, or at least 1000 days) following administration.

Alternatively or in addition, the NK cells of the population are optionally capable of inducing antibody-dependent cell-mediated cytotoxicity (ADCC) and/or natural cellular cytotoxicity (NCC) of a cancer cell. NK cells mediate NCC via adhesion and conjugation to target cells and release of perforin and granzymes, which induce apoptosis in the target cell. ADCC is a mechanism by which immune cells bearing the Fc receptor kill cells coated with antibody upon binding of the Fc receptor to the Fc portion of the antibody. NK cells mediate ADCC through, e.g., CD16, engagement of which results in release of cytolytic granules and inflammatory mediators. Methods of characterizing ADCC activity and NCC activity are known in the art and described herein and in, e.g., U.S. Patent Publication No. 2020/0131475 (which describes an assay involving use of Raji cells (derived from Burkett's lymphoma) preincubated with anti-CD20 antibody as targets for ADCC assays (Tsirigotis et al., J of Steroid Biochem and Mol Bio 108:267-271 (2008)); U.S. Patent Publication No. 2022/0040230; and Bhatnagar et al., Eur. J. Immunol., 44 (11): 3368 (2014). In various aspects, the administration of the population of NK cells to a subject suffering from cancer decreases tumor load or burden in the subject by at least 1%, at least 2%, at least 5%, at least 10%, at least 20%, at least 30%, at least 40%, at least 50%, at least 60%, at least 70%, at least 80%, at least 90%, or 100%. Tumor load may be characterized using any suitable method including, but not limited to, X-ray, computerized tomography (CT), magnetic resonance imaging (MRI), positron emission tomography (PET), ultrasound, endoscopy and laparoscopy, tumor marker levels, cytology, histology, biopsy sampling, and/or counting of target cells in circulation.

Optionally, at least a portion of the NK cells in the population (e.g., at least 10%, at least 20%, at least 30%, at least 40%, at least 50%, at least 60%, at least 70%, at least 80%, at least 90% or 100%) are larger in volume compared to peripheral blood NK cells. Methods of determining cell volume are known in the art, as illustrated in Model, Cytometry, 93 (3): 281 (2018), incorporated by reference in its entirety and particularly with respect to descriptions of methods of measuring cell volume. In various aspects of the disclosure, NK cells of the population have at least about a 5% increase in cell volume as compared to peripheral blood NK cells. For example, NK cells of the population have at least about a 10% increase or at least about a 15% increase in cell volume (e.g., about 5% to about 20%, about 7% to about 18%, about 10% to about 15%, about 10% to about 20%, or about 15% to about 20% increase) as compared to peripheral blood NK cells.

The disclosure also provides a kit comprising one or more containers comprising the population of NK cells described herein. The kit may further comprise instructions and written information on indications and usage of the NK cells. Sterile sealed containers, e.g., vials, bottle, vessel, and/or packages comprising the population of NK cells, optionally with suitable instructions for use, are also contemplated. In a further aspect, the disclosure provides an article of manufacture, or unit dose form, comprising: (a) the population of NK cells described herein; (b) a container containing said population; and (c) a label affixed to said container, or a package insert included in said container referring to the use of said NK cells in the treatment of a disease or disorder (e.g., cancer).

Also provided herein are pharmaceutical compositions comprising the population of NK cells and a pharmaceutically acceptable carrier, excipient, or diluent. In exemplary aspects, the composition is a sterile composition. The pharmaceutical composition according to the disclosure may be formulated for delivery via any route of administration. “Route of administration” may refer to any administration pathway known in the art, including but not limited to aerosol, nasal, oral, transmucosal, transdermal, or parenteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, intratumoral, transmucosal, or transtracheal. The composition may be in the form of a solution or suspension for infusion or for injection. Each component of the carrier is “pharmaceutically acceptable” in that it must be compatible with the other ingredients of the formulation and safe for use in vivo.

The disclosure further provides a method of treating a disease or disorder in a subject in need thereof, the method comprising administering to the subject an effective amount of the population of engineered NK cells or the pharmaceutical composition comprising the population of engineered NK cells, thereby treating the disease or disorder in the subject. Similarly, the disclosure provides use of the population of engineered NK cells in the treatment of a disease or disorder, as well as use of the population of NK cells in the preparation of a medicament for treating a disease or disorder in a subject in need thereof.

In various aspects, the disease or disorder is cancer. Examples of cancers include, but are not limited to, prostate cancer, lung cancer (e.g., non-small cell lung cancer), colon cancer, endometrial cancer, soft tissue carcinoma, rectum cancer, urinary bladder cancer, melanoma, kidney cancer, renal cancer (e.g., renal cell carcinoma), oral cavity cancer, pharynx cancer, pancreatic cancer, uterine cancer, thyroid cancer, parathyroid cancer, skin cancer, head and neck cancer (e.g., head and neck squamous cell carcinoma (HNSCC), cervical cancer, brain cancer (e.g., glioblastoma), liver cancer (e.g., hepatocellular carcinoma), bone cancer (e.g., osteosarcoma), or ovarian cancer. Hematopoietic cancers are particularly contemplated. Examples of cancers also include, but are not limited to, leukemias and lymphomas, such as acute myeloid leukemia, Hairy Cell Leukemia, Chronic Lymphocytic Leukemia, and Non-Hodgkin's Lymphoma (e.g., Diffuse Large B-cell Lymphoma, Burkitt Lymphoma, Mantel cell Lymphoma, and follicular lymphoma). Other cancers include, but are not limited to, alveolar rhabdomyosarcoma, breast cancer, cancer of the anus, anal canal, or anorectum, cancer of the eye, cancer of the intrahepatic bile duct, cancer of the joints, cancer of the nose, nasal cavity, or middle ear, esophageal cancer, gastrointestinal cancer, Hodgkin lymphoma, malignant mesothelioma, multiple myeloma, rectal cancer, renal cancer (e.g., renal cell carcinoma (RCC)), small intestine cancer, soft tissue cancer, stomach cancer, testicular cancer, and ureter cancer.

In various aspects, the disease or disorder is an autoimmune disease or disorder. Autoimmunity is an immune response to self-antigens, which results in the body attacking normal cells and tissues. There are more than 80 types of autoimmune diseases. The body's response to autoimmune conditions mimics the response against infection, involving activation of immune cells, inflammation, and tissue damage. In various aspects the autoimmune condition is one of over 80 diseases or syndromes hallmarked by the loss of B-cell tolerance and the production of autoantibodies. In various aspects, the autoimmune condition is Graft versus Host Disease (GvHD), systemic lupus erythematosus (SLE), multiple sclerosis, Sjogren's syndrome, systemic sclerosis/scleroderma, cutaneous sclerosis, ulcerative colitis, inflammatory bowel disease, bullous pemphigus, insulin dependent diabetes, mellitus or Type 1 diabetes, Crohn's disease, psoriatic arthritis or rheumatoid arthritis.

The method may comprise administering the population of NK cells prior to, simultaneously with, or following, a different therapy, such as chemotherapy, surgical resection of a tumor, immunotherapy (such as cytokine therapy or administration of an antibody construct), or radiation therapy. For example, the disclosure contemplates a method wherein the population of engineered NK cells described herein is administered to a subject in need thereof as part of a treatment regimen that also comprises administration of one or more cytokines to the subject. In some aspects, the cytokine is an interleukin (e.g., IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20. IL-21, IL-22, IL-23, IL-24, IL-25, IL-26, IL-27, IL-28, IL-29, IL-30, IL-31, IL-32, or IL-33). For example, in various aspects of the method, the method further comprises administering IL-15 and/or IL-2 to the subject.

In various aspects, the method may comprise administering the population of engineered NK cells described herein (such as FcR-UDC I-NKs (e.g., differentiated cells having a disruption in the B2M gene resulting in the abrogation of the HLA class I protein and expressing an exogenous single chain fusion HLA-E as well as CD16) or FcR-UDC I/II-NKs (e.g., FcR-UDC I-NKs further engineered with a knockout mutation of a HLA class II related gene, such as RFXANK, resulting in disruption of the HLA class II protein) as part of a treatment regimen which also comprises administration of one or more antibody constructs (e.g., antibodies) to the subject. The disclosure further contemplates a treatment regimen wherein an antigen binding antibody fragment or an antibody-like protein product (i.e., a protein based on antibody structural features that allow antigen binding) is administered to a subject. “Antibody construct” refers to antibodies, antigen-binding fragments of antibodies, or antibody-like protein products. “Antibody” refers generally to an intact immunoglobulin. Disclosure herein referencing antibodies also applies to antibody fragments and antibody-like protein products. Suitable antigen-binding antibody fragments include, e.g., a F(ab)2 or a Fab fragments. Antibody-like protein products include, but are not limited to, single chain Fvs (scFv), diabodies, nanobody constructs, antibody mimetics, and the like. In various aspects, the method comprises administering an intact antibody or antibody fragment or antibody-like protein product that comprises an Fc region. In various aspects, the antibody, antibody fragment, or antibody-like protein product binds a cell surface antigen (e.g., a tumor antigen), such as, but not limited to, 5T4, ACE, ADRB3, AKAP-4, ALK, Androgen receptor, AOC3, APP, Axin1, AXL, B7H3, B7-H4, BCL2, BCMA, bcr-abl, BORIS, BST2, C242, C4.4a, CA125, CA6, CA9, CAIX, CCL11, CCR5, CD123, CD133, CD138, CD142, CD15, CD15-3, CD171, CD179a, CD18, CD19, CD19-9, CD2, CD20, CD22, CD23, CD24, CD25, CD27L, CD28, CD3, CD30, CD31, CD300LF, CD33, CD352, CD37, CD38, CD4, CD40, CD41, CD44, CD44v6, CD5, CD51, CD52, CD54, CD56, CD62E, CD62P, CD62L, CD70, CD71, CD72, CD74, CD79a, CD79b, CD80, CD90, CD97, CD125, CD138, CD141, CD147, CD152, CD154, CD326, CEA, CEACAM5, CFTR, CLDN3, CLDN18.2, CLDN18.1, CLDN6, CLEC12A, CLL-1, cll3, c-MET, Crypto 1 growth factor, CS1, CTLA-4, CXCR2, CXORF61, Cyclin B1, CYP1B1, Cadherin-3, Cadherin-6, DLL3, E7, EDNRB, EFNA4, EGFR, EGFRVIII, ELF2M, EMR2, ENPP3, EPCAM, EphA2, Ephrin A4, Ephrin B2, EPHB4, ERBB2 (Her2/neu), ErbB3, ERG (TMPRSS2 ETS fusion gene), ETBR, ETV6-AML, FAP, FCAR, FCRL5, FGFR1, FGFR2, FGFR3, FGFR4, FLT3, Folate receptor alpha, Folate receptor beta, FOLR1, Fos-related antigen 1, Fucosyl GM1, GCC, GD2, GD3, GM3, GPC1, GPC2, GPC3, GPNMB, GPR20, GPRC5D, GUCY2C, HAVCR1, HER2, HER3, HGF, HMI.24, HMWMAA, HPV E6, hTERT, ICAM, ICOS-L, IGF-I receptor, IGLL1, IL-2 receptor, IL-4 receptor, IL-13Ra2, IL-1 1Ra, interferon receptor, integrins (including α₄, αᵥβ₃, αᵥβ₅, αᵥβ₆, α₁β₄, α₄β₁, α₄≢₇, α₅β₁, α₆β₄, α_{11b}β₃ integrins), KIT, LAGE-la, LAIR1, LAMP-1, LCK, LFA-1 (CD11a), L-selectin (CD62L), LILRA2, LIV-1, LMP2, LRRC15, LY6E, LY6K, LY75, MAD-CT-1, MAD-CT-2, MAGE A1, MelanA/MART1, Mesothelin, ML-IAP, MSLN, mucin, MUC1, MUC16, MYCN, myostatin, NA17, NCA-90, NCAM, NOTCH1, NOTCH2, NOTCH3, NOTCH4, NY-BR-1, NY-ESO-1, OR51E2, OY-TES1, p53, p53 mutant, PANX3, PAP, PAX3, PAX5, p-CAD, PD-L1, PD-L2, PDGFR, PDGFR-beta, PIK3CA, PLAC1, Polysialic acid, PRSS21, PSCA, PSMA, PTK7, RAGE-1, STEAP1, TAG72, TARP, TCRB, TEM1/CD248, TEM7R, TGF-1, TGF-β2, TGS5, Tie 2, TIM-1, Tn Ag, TRAC, TRAIL-R1, TRAIL-R2, TROP-2, TRP-2, TRPV1, TSHR, tumor antigen CTAA16.88, UPK2, VEGF, VEGFR1, VEGFR2, vimentin, WT1, XAGE1, or combinations thereof. Examples of antibodies for use in connection with the method disclosed herein include, but are not limited to, Rituximab, Cetuximab, Trastuzumab, Panitumumab, Ofatumumab, Brentuximab, Pertuzumab, Ado-trastuzumab emtansine, Obinutuzumab, Nimotuzumab, Bevacizumab, Alemtuzumab, Gemtuzumab, Ranibizumab, Olaratumab, Ontuximab, Isatuximab, Sacituzumab, Daratumumab, Lintuzumab, Balantamab, Indatuximab, Dinutuximab, Alemtuzumab, Ibritumomab, Tositumomab, Panitumumab, Tremelimumab, Ticilimumab, Catumaxomab, Oregovomab, Zolbetuximab (described in, e.g., International Patent Publication No. WO 2016166124, hereby incorporated by reference in its entirety and particularly with respect to the description of Zolbetuximab), ASP1650 (described in, e.g., International Patent Publication No. WO 2012156018, hereby incorporated by reference in its entirety and particularly with respect to the description of SEQ ID NOs: 35 and 36, CDR sequences within SEQ ID NOs: 35 and 36, and SEQ ID NOs: 27 and 25), ASP1893 mAb (a humanized version of ASP1650 (i.e., sharing the CDR sequences of ASP1650)), and Veltuzumab. Additional examples of antibodies include, but are not limited to, Adalimumab, Eculizumab, and Natalizumab. In some aspects, the antibody is Rituximab, ASP 1893 mAb, ASP1650, or Zolbetuximab.

For example, the disclosure provides a method of treating a disease or disorder in a subject in need thereof, the method comprising administering to the subject an effective amount of the population of engineered NK cells described herein and further administering to the subject an antibody which binds a cell surface antigen on a target cell, the elimination of which provides a therapeutic benefit to the subject. In various aspects, the disclosure provides a method of treating cancer in a subject in need thereof, wherein the method comprises administering to the subject an effective amount of the population of engineered NK cells and an effective amount of antibody specific for a cancer cell antigen (such as any of the anti-cancer antibodies described herein). In various aspects of the disclosure, an effective amount refers to treating cancer in a patient by administering multiple doses and/or multiple rounds of doses of NK cells and antibodies as a treatment course. As merely examples of aspects of the disclosure, the subject may suffer from breast cancer, and the NK cells are administered with an anti-HER2 antibody (such as Trastuzumab); or the subject may suffer from colon carcinoma, and the NK cells are administered with an anti-EGFR antibody (such as cetuximab or panitumumab); or the subject may suffer from AML, and the NK cells are administered with an anti-CD123 antibody or an anti-FLT3 antibody. The subject may suffer from a CD20-positive cancer. In various aspects, the subject is suffering from a CLDN18.2-positive cancer, such as gastric or gastroesophageal junction (GEJ) adenocarcinoma, and the NK cells are administered with an anti-CLDN18.2 antibody. Alternatively, the subject may be suffering from a CLDN6-positive cancer (e.g., ovarian cancer, in particular ovarian adenocarcinoma or ovarian teratocarcinoma, lung cancer, including small cell lung cancer (SCLC) and non-small cell lung cancer (NSCLC), in particular squamous cell lung carcinoma and adenocarcinoma, gastric cancer, breast cancer, hepatic cancer, pancreatic cancer, skin cancer, in particular basal cell carcinoma or squamous cell carcinoma, malignant melanoma, head and neck cancer, in particular malignant pleomorphic adenoma, sarcoma, in particular synovial sarcoma or carcinosarcoma, bile duct cancer, cancer of the urinary bladder, in particular transitional cell carcinoma and papillary carcinoma, kidney cancer, in particular renal cell carcinoma including clear cell renal cell carcinoma or papillary renal cell carcinoma, colon cancer, small bowel cancer, including cancer of the ileum, in particular small bowel adenocarcinoma and adenocarcinoma of the ileum, testicular embryonal carcinoma, placental choriocarcinoma, cervical cancer, testicular cancer, in particular testicular seminoma, testicular teratoma and embryonic testicular cancer, uterine cancer, a germ cell tumor such as a teratocarcinoma or an embryonal carcinoma, in particular a germ cell tumor of the testis, and the metastatic forms thereof), and the NK cells are administered with an anti-CLDN6 antibody. Optionally, the antibody is Rituximab, Zolbetuximab, ASP1893 mAb, or ASP1650.

In various aspects, the disclosure provides a method of treating an autoimmune condition in a subject in need thereof, wherein the method comprises administering to the subject an effective amount of the population of engineered NK cells and an effective amount of antibody specific for an immune cell antigen. Optionally, the antibody is Rituximab.

In some aspects of the disclosure, the engineered NK cells are optionally co-administered with an antibody as part of a treatment regime which may optionally include a chemotherapeutic treatment that is not an antibody. Optionally, the chemotherapeutic agent is selected from fluorouracil, oxaliplatin, and/or capecitabine.

As used herein, the term “treat,” as well as words related thereto, do not necessarily imply 100% or complete treatment or remission. Rather, there are varying degrees of treatment of which one of ordinary skill in the art recognizes as having a potential benefit or therapeutic effect. In this respect, the methods of treating a disease or disorder can provide any amount or any level of treatment. Furthermore, the treatment provided by the method may include treatment of one or more conditions or symptoms or signs of the disease being treated. For instance, the treatment method of the present disclosure may inhibit one or more symptoms of the disease. Also, the treatment provided by the methods of the present disclosure may encompass slowing the progression of the disease.

Treatment for cancer may be determined by any of a number of ways. Any improvement in the subject's wellbeing is contemplated (e.g., at least or about a 10% reduction, at least or about a 20% reduction, at least or about a 30% reduction, at least or about a 40% reduction, at least or about a 50% reduction, at least or about a 60% reduction, at least or about a 70% reduction, at least or about an 80% reduction, at least or about a 90% reduction, or at least or about a 95% reduction of any parameter described herein). For example, a therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth or appearance of new lesions; (6) decrease in tumor size or load (i.e., burden); (7) absence of clinically detectable disease, (8) decrease in levels of cancer markers; (9) an increased patient survival rate or survival time; and/or (10) some relief from one or more symptoms associated with the disease or condition (e.g., pain).

For example, in various aspects, administration of the engineered NK cells of the disclosure increases survival time of the subject by at least 20 days (e.g., at least 30 days, at least 45 days, at least 60 days, at least 90 days, or more) relative to survival time of the subject in the absence of administering the population of engineered NK cells. Alternatively or in addition, administering the NK cells to the subject decreases tumor load in the subject. In various aspects, the methods of the disclosure further comprise monitoring treatment in the subject. Disease states may be monitored by, e.g., clinical examination, X-ray, computerized tomography (CT, such as spiral CT), magnetic resonance imaging (MRI), positron emission tomography (PET), ultrasound, endoscopy and laparoscopy, tumor marker levels in the context of cancer (e.g., carcinoembryonic antigen (CEA)), cytology, histology, biopsy sampling, and/or counting of target cells in circulation. Other methods and criteria known in the field include, e.g., RECIST (Response Evaluation Criteria In Solid Tumors) and irRC (immune response criteria). The term “effective amount” refers to a sufficient quantity of NK cells (or co-therapy, such as antibody therapy) to achieve a desired biological response in a clinically relevant time period; for example, “therapeutically effective” refers to a sufficient quantity of NK cells which ameliorates one or more causes or symptoms of a condition or disease. The amount of engineered NK cells administered to a subject may comprise, e.g., a dose of 10⁴ to 10¹¹, 10⁵ to 10¹⁰, or 10⁶ to 10⁹ cells/kg body weight.

The subject is a mammal, including, but not limited to, mammals of the order Rodentia, such as mice and hamsters; mammals of the order Logomorpha, such as rabbits; mammals from the order Carnivora, including Felines (cats) and Canines (dogs); mammals from the order Artiodactyla, including Bovines (cows) and Swines (pigs); or of the order Perssodactyla, including Equines (horses). In some aspects, the mammal is of the order Primate, Ceboid, or Simoid (monkey) or of the order Anthropoid (humans and apes). In some aspects, the mammal is a human. The terms “subject in need thereof” include subjects already afflicted with a disease or disorder, as well as those in which the disease or disorder is to be prevented.

The disclosure also provides a method of producing pluripotent stem (PS) cell-derived natural killer (NK) cells under feeder free, optionally serum-free, conditions. The method comprises (a) culturing pluripotent stem cells, optionally the PSCs are genetically engineered to disrupt one or more copies (e.g., all copies) of endogenous B2M and/or one or more copies (e.g., all copies) of a HLA class II-related gene selected from the group consisting of RFXANK, RFX5, RFXAP, and CIITA, and optionally expresses an engineered non-classical HLA class I protein associated with B2M, e.g., HLA-E; (b) differentiating the pluripotent stem cells to precursor cells capable of differentiating to hematopoietic cell types, endothelial cell types, and/or mesodermal derivatives; and (c) differentiating the precursor cells to NK cells under feeder free, optionally serum-free, conditions in a culture media comprising SCF, IL-7, IL-15, and Flt3L (Flt3 ligand). In various aspects, step (b) comprises differentiating the pluripotent stem cells using embryoid bodies to obtain precursor cells capable of differentiating to hematopoietic cell types, endothelial cell types, and/or mesodermal derivatives. In various aspects, the PSC is a human PSC. In other various aspects, the method further comprises (d) culturing the NK cells in a culture media comprising IL-15 and IL-18 (and, optionally, IL-21). In various aspects, step (d) comprises culturing the NK cells in the presence of an antibody or ligand that binds a receptor on NK cells to promote activation and/or expansion of the NK cells. In various aspects, the antibody or ligand binds to DNAM-1, OX40, NKG2D, 2B4, NKp30, or NKp46. Optionally, the method comprises culturing the NK cells in the presence of an antibody that binds DNAM-1, an antibody that binds OX40, an antibody that binds NKG2D, an antibody that binds 2B4, an antibody that binds NKp30, and/or an antibody that binds NKp46; a combination of any of the foregoing antibodies may be provided during the culturing step. Alternatively, the method may comprise culturing the NK cells in the presence of a ligand that binds DNAX Accessory Molecule 1 (DNAM-1), a ligand that binds OX40 (e.g., OX40L), a ligand that binds Natural Killer Group 2D (NKG2D) (e.g., MHC class I chain-related protein A (MICA) or UL16 binding protein 1 (ULBP1)), a ligand that binds 2B4 (CD244), a ligand that binds Natural Killer Protein 30 (NKp30) (e.g., B7H6), and/or a ligand that binds Natural Killer Protein 46 (NKp46, CD355), to activate and/or expand the NK cell. A combination of any of the foregoing ligands may be provided during the culturing step. Optionally, the ligand comprises at least the portion of a native ligand that mediates receptor binding and activation fused to an Fc domain.

In various aspects, the method described herein is particularly well suited for the large-scale production of human PSC-derived NK cells. Previous methods of NK production under feeder-free conditions allowed up to 5- to 500-fold expansion of PBNK cells. See, e.g., Lu et al., J. Hematol. Oncol. 14:7 (2021). Remarkably, the method of the disclosure allows for at least a 1000-fold expansion of cells. Indeed, the method of the disclosure allows, in some embodiments, at least a 2000-fold, at least a 3000-fold, at least a 4000-fold, at least a 5000-fold, at least a 6000-fold, at least a 7000-fold, at least an 8000-fold, at least a 9000-fold, or at least a 10,000-fold expansion (or any range therein having these values as endpoints). The level of expansion achieved under feeder-free conditions using the materials and methods described herein is superior to previous methods.

The method of producing human PSC-derived engineered NK cells is performed under “feeder free conditions.” “Feeders” are cells often co-cultured with cells of interest, e.g., PSC and/or NK cells; feeder cells which release growth factors and nutrients into the cellular milieu which support and promote target cell (e.g., PSC) growth. Cell types used as feeder cells include, e.g., fibroblasts (mouse or human), as well as endothelial cells and other stromal cells (e.g., OP9 cells). Feeder free conditions refers to culture conditions (or media) essentially free of feeder (e.g., stromal) cells and/or which has not been pre-conditioned by the cultivation of feeder cells. The method of the disclosure surprisingly provides a robust population of NK cells without the need for feeder cells or media conditioned by feeder cells. Indeed, even when compared to methods of NK production under feeder condition, the method of this disclosure allows for comparable to NK production. The use of feeder-free condition is cost-effective and minimizes xenogeneic contaminants.

“Culturing” refers to maintaining a cell in culture medium under conditions suitable for the survival and/or proliferation of the cell. The steps described herein may be performed using any suitable vessel (e.g., microwell plate, flask, bioreactor, etc.) and environmental conditions which permit cell growth and differentiation. Any suitable cell culture media for growth and differentiation of stem cells may be used in the method. Examples of media include, but are not limited to, mTeSR™1, TeSR™2, mTeSR™ Plus, TeSR™E8, APEL™, STEMdiff™ APEL™2, or StemSpan™ from STEMCELL Technologies (Vancouver, Canada), primate ES/IPS cell medium from ReproCELL (Boston, MA), and StemPro®-34 and StemPro® hESC SFM from Invitrogen (Carlsbad, CA), Gibco® PFHM-II media from ThermoFisher Scientific (Waltham, MA), and X-VIVO™ from Lonza (Basel, Switzerland). The media optionally comprises supplements such as, for example, amino acids (e.g., glutamine, arginine, or asparagine), hydrolysate, vitamins, nucleosides, hormones and/or other growth factors (such as insulin, transferrin, insulin-like growth factor, or epidermal growth factor), ions (e.g., sodium, chloride, calcium, or magnesium), buffers, small molecules (e.g., a GSK-3 inhibitor, a MEK inhibitor, or a Rho Kinase (ROCK) inhibitor), and/or sugar(s) (e.g., glucose or galactose). Medium lacking one or more of these supplements also is contemplated. In various aspects, the media is chemically defined media, i.e., cell culture media in which all of the chemical components are known, and which typically does not include serum, serum albumin, and hydrolysate.

The method comprises culturing human pluripotent stem cells, optionally, genetically engineered to disrupt one or more copies (e.g., all copies) of endogenous B2M and/or one or more copies (e.g., all copies) of RFXANK, RFX5, RFXAP, or CIITA (or a combination thereof), and optionally express an engineered non-classical HLA class I protein associated with B2M, e.g., HLA-E, as described above with respect to the generation of a population of NK cells. The disclosure relating to PSCs and NK cells provided above also apply to the PSC for use in the method. For example, the human PSCs optionally comprise a nucleic acid molecule encoding one or more suicide gene products which may be present in one or more copies, such as HSV thymidine kinase, and are optionally embryonic stem cells or induced PS cells, as described above.

Any suitable initial seeding density may be employed to establish a culture for differentiation. For example, an initial seeding density of about 0.5×10⁵ to about 10×10⁵ human pluripotent stem cells/mL may be used, (e.g., about 1×10⁵ to about 5×10⁵ or about 1.5×10⁵ to about 3.5×10⁵ or about 2×10⁵ to about 2.5×10⁵ cells/mL). The human pluripotent stem cells may be cultured for any suitable amount of time before the first differentiation step, e.g., one to seven days (such as one to four days or one to three days), to provide a sufficient number of human pluripotent stem cells suitable for differentiation. The method then comprises differentiating the human pluripotent stem cells to generate precursor cells capable of differentiating to hematopoietic cell types, endothelial cell types, and/or mesodermal derivatives. In various aspects, the pluripotent stem cells are differentiated to precursor cells using embryoid bodies (EBs). See, e.g., Itskovitz-Eldor et al., Mol Med., 6 (2): 88-95 (2000), hereby incorporated by reference in its entirety. EBs are aggregates or clusters of multipotent cells. EBs may be employed as an intermediate stage in the production of precursor cells with the potential of further differentiating into hematopoietic cell types, endothelial cell types, and/or mesodermal derivatives (or as an intermediate stage in the production of hematopoietic cell types, endothelial cell types, and/or mesodermal derivatives themselves). In various aspects, the method does not comprise differentiating the human pluripotent stem cells to hemangioblast or hemangio-colony forming cells as precursor cells.

The differentiation step to produce precursor cells comprises culturing the human PSCs under conditions suitable for producing the precursor cells (e.g., using EBs). In exemplary aspects, the human pluripotent stem cells are cultured in media comprising cytokines that promote the formation of EBs. In various aspects, the media comprises at least one of bone morphogenic protein 4 (BMP4), vascular endothelial growth factor (VEGF), or stem cell factor (SCF). Combinations of BMP4, SCF, and VEGF are contemplated, including use of all three cytokines to induce differentiation. The cytokines may be used in any suitable amount. For example, about 1 ng/ml to about 40 ng/ml of BMP4 may be provided (e.g., about 5 ng/ml to about 35 ng/ml, about 10 ng/ml to about 30 ng/ml, about 15 ng/ml to about 25 ng/ml, about 16 ng/ml, about 17 ng/ml, about 18 ng/mL, about 19 ng/mL, about 20 ng/ml, about 21 ng/ml, about 22 ng/ml, about 23 ng/ml, or about 24 ng/mL of BMP4 may be provided). Optionally, about 1 ng/ml to about 40 ng/ml of VEGF may be provided (e.g., about 5 ng/ml to about 35 ng/ml, about 10 ng/ml to about 30 ng/ml, about 15 ng/ml to about 25 ng/ml, about 16 ng/ml, about 17 ng/ml, about 18 ng/ml, about 19 ng/ml, about 20 ng/ml, about 21 ng/ml, about 22 ng/ml, about 23 ng/ml, or about 24 ng/ml of VEGF is provided). Also, optionally, about 10 ng/ml to about 60 ng/ml of SCF may be provided (e.g., about 25 ng/ml to about 55 ng/ml, about 30 ng/ml to about 50 ng/ml, about 35 ng/mL to about 45 ng/ml, about 36 ng/ml, about 37 ng/ml, about 38 ng/ml, about 39 ng/ml, about 40 ng/ml, about 41 ng/mL, about 42 ng/ml, about 43 ng/ml, or about 44 ng/ml of SCF is provided). In various aspects, the method comprises exposing the human pluripotent stem cells to fibroblast growth factor (FGF, such as bFGF); alternatively, the method does not comprise the use of FGF (e.g., bFGF). The differentiation step may be performed for any suitable amount of time, e.g., about one day to about 21 days, such as about one day to about 14 days, about 7 days to about 18 days, about 10 days to about 14 days, or about 14 days. In various aspects, the first differentiation step is performed up to about 14 days. Media may be changed periodically during the differentiation step culture period. Generally, at least a portion of the media (e.g., half the media) is exchanged every other day until the end of the differentiation step. The conditions described herein result in high quality EBs, which lead to improved differentiation and NK cell yield.

The method further comprises differentiating the precursor cells (e.g., using EBs) to NK cells under feeder free, optionally serum-free, conditions in a culture media comprising SCF, IL-7, IL-15, and FIt3L (i.e., step (c) or second differentiation step). In various aspects, precursor cells are harvested and replated in a vessel suitable for growth, expansion, and differentiation. The harvested cells are cultured in media comprising SCF (e.g., about 5 ng/ml to about 40 ng/ml, about 10 ng/ml to about 35 ng/ml, about 15 ng/ml to about 30 ng/ml, about 15 ng/ml to about 25 ng/ml, about 16 ng/ml, about 17 ng/ml, about 18 ng/ml, about 19 ng/ml, about 20 ng/ml, about 21 ng/ml, about 22 ng/ml, about 23 ng/ml, or about 24 ng/ml of SCF), IL-7 (e.g., about 5 ng/ml to about 40 ng/mL, about 10 ng/ml to about 35 ng/ml, about 15 ng/ml to about 30 ng/ml, about 15 ng/ml to about 25 ng/ml, about 16 ng/ml, about 17 ng/ml, about 18 ng/ml, about 19 ng/ml, about 20 ng/ml, about 21 ng/ml, about 22 ng/ml, about 23 ng/ml, or about 24 ng/ml of IL-7), IL-15 (e.g., about 1 ng/ml to about 30 ng/ml, about 5 ng/ml to about 20 ng/ml, about 7 ng/ml to about 15 ng/ml, about 9 ng/ml to about 12 ng/ml, about 10 ng/ml of IL-15), and Flt3L (e.g., about 1 ng/ml to about 30 ng/ml, about 5 ng/ml to about 20 ng/mL, about 7 ng/ml to about 15 ng/ml, about 9 ng/ml to about 12 ng/ml, or about 10 ng/ml of Flt3L). The second differentiation step may be performed for any suitable amount of time, e.g., about one day to about 35 days, such as one day to about 21 days, about 7 days to about 32 days, about 10 days to about 28 days, about 14 days to about 25 days, or about 21 days. In various aspects, this step is performed up to about 21 days. Media may be changed periodically during the second differentiation step culture period. In some aspects, the culture is not disturbed for an initial period of time (e.g., one, two, three, or four days calculated from the start of the differentiation step), although this is not required. At least a portion of the media (e.g., half the media) may be exchanged every other day, twice a week, once a week, etc. until the end of the differentiation step.

In various aspects, IL-3 is included in the media during the second differentiation step. In this regard, about 1 ng/ml to about 10 ng/ml of IL-3 is optionally provided (e.g., about 3 ng/ml to about 8 ng/ml, about 4 ng/ml to about 6 ng/ml, about 2 ng/ml, about 3 ng/ml, about 4 ng/ml, about 5 ng/ml, about 6 ng/ml, about 7 ng/ml, about 8 ng/ml, or about 9 ng/ml of IL-3 is provided). IL-3 is optionally only provided for a portion of the second differentiation step culture period, e.g., only for the first one, two, three, four, five, six, or seven days of culture, and is not included in the media for the remaining time of the second (NK) differentiation step.

In a preferred aspect of the disclosure, the culture media of the second differentiation step (step (c), differentiation of precursor cells to NK cells) comprises UM171. Any suitable amount of UM171 may be employed to achieve a desired effect; typically about 10 nM to about 60 nM (e.g., about 15 nM to about 55 nM, about 20 nM to about 50 nM, about 25 nM to about 45 nM, about 30 nM to about 40 nM, about 31 nM, about 32 nM, about 33 nM, about 34 nM, about 35 nM, about 36 nM, about 37 nM, about 38 nM, or about 39 nM) is employed. UM171 is a pyrimido-indole derivative that significantly increases the yield of NK cells (i.e., NK cell expansion) and reduces variability in the resulting NK population. Thus, a substantially homogenous population of NK cells can be achieved without the need for purification or filtration of the final NK cell product. The term “homogenous” refers to a population of cells wherein each cell is the same or substantially the same as the other cells in the population (i.e., expresses one or more of the same cell surface markers indicative of NK cells, such as CD56, CD45, CD25, DNAM-1, NKp30, NKG2D, and/or NKp44). In various aspects of the method, at least 90%, at least 95%, at least 96%, at least 97%, at least 98%, at least 99%, or more of the cells of the resulting human pluripotent stem cell-derived engineered NK cell population are the same or substantially the same as other cells in the population (e.g., display one or more of the cell surface markers above). In this regard, the disclosure contemplates a method which does not comprise a purification, isolation, or a filtration step following differentiation of the precursor cells to NK cells (or optionally following the step of culturing the NK cells to promote activation) to remove undesired cell types. In various aspects, UM171 is present only in the culture media of step (c) and is not present in the culture media of any other step(s) of the method (although this is not required).

The method of the disclosure optionally comprises a further culture step (step (d) to promote activation and/or expansion of the NK cells. While this step is not required, it may be desirable to provide a population of NK cells with particularly desirable characteristics (e.g., improved cytotoxicity, improved persistence in vivo, improved engraftment, etc.). The activation step comprises culturing the NK cells (from the second differentiation step, step (c)) in a culture media comprising IL-15 and IL-18. IL-15 and IL-18 may be provided in any suitable amount, including, for example, about 1 ng/ml to about 30 ng/ml, about 5 ng/ml to about 20 ng/ml, about 7 ng/ml to about 15 ng/ml, about 9 ng/ml to about 12 ng/ml, or about 10 ng/ml of each of IL-15 and IL-18. Optionally, the NK cells are cultured in the presence of IL-15, IL-18, and IL-21 during at least a portion of the activation step (step (d)). Suitable amounts of IL-21 include, but are not limited to, about 1 ng/ml to about 30 ng/mL, about 5 ng/ml to about 20 ng/ml, about 7 ng/ml to about 15 ng/ml, about 9 ng/ml to about 12 ng/ml, or about 10 ng/ml of IL-21). In various aspects, culture media utilized in the activation step does not comprise IL-12.

In various aspects, the NK cells are cultured in the presence of an antibody that binds to DNAM-1 (DNAX accessory molecule, CD222), an antibody that binds to OX40, an antibody that binds to NKG2D, an antibody that binds to 2B4, an antibody that binds to NKp30, an antibody that binds to NKp46, or a combination of any of the foregoing antibodies. For example, step (d) may comprise culturing the NK cells in the presence of an antibody that binds NKp30. NKp30 is also referenced as CD337 and is a 30 kD type I transmembrane protein which is a stimulatory receptor on NK cells. Alternatively or in addition, step (d) may comprise culturing the NK cell in the presence of an antibody that binds 2B4. 2B4, also referenced as CD244, is a member of the signaling lymphocyte activation molecule (SLAM) family that mediates non-MHC restricted killing. Alternatively or in addition, step (d) may comprise culturing the NK cell in the presence of an antibody that binds DNAM-1. DNAM-1 is also referenced as CD226 and is a 65 kD glycoprotein member of the Ig-superfamily expressed on the surface of NK cells, T cells, and monocytes/macrophages. For example, step (d) may comprise culturing the NK cell in the presence of an antibody that binds NKp30 and an antibody that binds 2B4. In a further example, step (d) may comprise culturing the NK cell in the presence of an antibody that binds NKp30 and an antibody that binds DNAM-1. Anti-NKp30 antibodies, 2B4 antibodies, and anti-DNAM-1 antibodies are commercially available from, e.g., SinoBiological (Wayne, PA) and BioLegend (San Diego, CA). In various aspects, step (d) may comprise culturing the NK cells in the presence of a ligand that binds DNAM-1, a ligand that binds OX40 (e.g., OX40L), a ligand that binds NKG2D (e.g., MICA or ULBP1), a ligand that binds 2B4 (CD244), a ligand that binds NKp30 (e.g., B7H6), and/or a ligand that binds NKp46, to activate the NK cell. A combination of any of the foregoing ligands may be provided during the culturing step. The antibody, antibodies, ligand, or ligands may be provided in the culture media or may be coated on the vessel.

The activation step (step (d)) may be performed for any suitable amount of time, e.g., one, two, three, four, five, six, seven, eight, nine, 10, 11, 12, 13, or 14 days. In various aspects, step (d) is performed for about five days to about 10 days. Additionally, the activation step may be performed in multiple vessels to accommodate proliferation of the NK cell population. For example, the activation step may comprise culturing the NK cells in one vessel (e.g., a flask, such as T75 flask) for an initial period of time (e.g., one, two, three, or four days), then transferring the cells to a larger vessel (e.g., a bioreactor) for a second period of time (e.g., one, two, three, or four days). Also, in various aspects, IL-21 is provided for only a portion of the activation step, when present at all. In this regard, the method may comprise inclusion of IL-21 in the culture media for an initial period time (e.g., one or more of days 0-4 of the activation step), but not include IL-21 in the culture media for the remainder of the culture period.

The NK cells are cultured in a vessel (e.g., flask or bioreactor) suitable for growth, expansion, and activation of NK cells. The vessel is optionally coated with a substance which promotes cell adhesion or one or more matrix component, such as laminin or laminin fragments, entactin, collagen, gelatin, vitronectin, fibronectin, Synthemax® (Corning Incorporated), Matrigel®, polylysine, thrombospondin, ProNectin-F™, and the like. An exemplary matrix component is RetroNectin®, a 63-kD fragment of recombinant human fibronectin fragment also referenced in the art as rFN-CH-296. Optionally, a Notch ligand is provided, such as DLL4. In various aspects, the vessel is coated with a Notch ligand, e.g., DLL4, and RetroNectin®. The DLL4 and RetroNectin® may be provided in any suitable amount, including relative amounts resulting in a DLL4:RetroNectin® ratio of 5:1, 4:1, 3:1, 2:1, 1:1, 1:2, 1:3, 1:4, or 1:5. Coated vessels may be employed for any of the method steps disclosed herein, including step (c) and/or step (d).

Remarkably, it has been determined that the activation step (step (d) may be performed multiple times without negatively impacting the characteristics of the NK cell population (e.g., without significantly reducing the functionality of the cells). If desired, step (d) may be repeated up to five times (i.e., repeated twice, three times, four times, or five times) to further expand and/or activate the NK cells.

In various aspects of the method, IL-2 and/or IL-6 is not included in the culture media of one or more (e.g., any) steps of the method.

The disclosure further provides a method of increasing the purity of a population of NK cells derived from pluripotent stem cells. The method comprises differentiating embryoid bodies (i.e., differentiating cells within embryoid bodies) to NK cells under feeder free, optionally serum-free, conditions in a culture media comprising SCF, IL-7, IL-15, and FIt3L and in the presence of UM171. In various aspects, the culture media further comprises IL-3. Optionally, the method is performed in a vessel coated with RetroNectin® and DLL4. Also optionally, the method is performed for about one day to about 21 days. The result of the method is a substantially pure population of CD56+, CD45+NK cells (i.e., a population of NK cells with low levels of unwanted cell types (contaminating cells). The method allows for the production of PSC-derived NK cells without the need for extensive further purification of the resulting cell population to obtain a substantially pure population of NK cells. In various aspects, the method results in a population of cells wherein at least about 75% of the cells are CD56+, CD45+NK cells (e.g., at least about 80%, at least about 85%, at least about 90%, at least about 95%, at least about 96%, at least about 97%, at least about 98%, or at least about 99% of the cells are CD56+CD45+ NK cells).

The method of the disclosure results in a robust population of NK cells suitable for use in, e.g., therapeutic applications. For example, the NK cells optionally express one or more cell surface markers selected from the group consisting of CD56, CD45, CD25, DNAM-1, NKp30, NKG2D, and NKp44, including any combination of the cell surface markers. In a preferred aspect, the NK cells express at least CD56. The NK cells also optionally express an engineered non-classical HLA class I protein associated with B2M, e.g., HLA-E, HLA-F, and/or HLA-G, as described above. The NK cells also may be engineered to express a CAR (e.g., a universal CAR), a FcR, a non-natural NKG2D receptor, or other type of receptor (e.g., as described above). The resulting NK cell population also preferably exhibits the characteristics described above, e.g., are capable of engrafting into a subject following administration and optionally remaining engrafted for at least 20 days, capable of inducing ADCC and/or NCC of a cancer cell, and capable of decreasing tumor load. In various aspects, the method results in production of at least 10⁹, at least 10¹⁰, or at least 10¹¹ CD56-positive NK cells.

The following example is given merely to illustrate the presently disclosed subject matter and not in any way to limit its scope.

### EXAMPLES

The examples describe exemplary methods of producing human pluripotent stem cell-derived engineered natural killer (NK) cells under feeder-free, serum-free conditions.

### Example 1: Engineered Human Pluripotent Stem Cells

Human pluripotent stem cells (PSCs) (NIH2, NIH3 and W1C1) were genetically engineered to (i) disrupt the B2M gene resulting in the abrogation of the HLA class I protein, and (ii) express an exogenous single chain fusion HLA-E (“UDC I-PSCs”). The UDC I-PSCs were further genetically engineered with a knockout mutation of a HLA class II related gene, RFXANK, resulting in disruption of the HLA class II protein (“UDC I/II-PSCs”). Alternatively, the UDC I-PSCs or UDC I/II-PSCs were further genetically engineered to express a gene encoding an Fc receptor/CD16 (“FcR-UDC I-PSCs” or “FcR-UDC I/II-PSCs”). UDC I-PSCs, UDC I/II-PSCs, and/or FcR-UDC I-PSCs are collectively referred to herein as “UDC PSCs.” Differentiated cells are referenced herein as, e.g., “FcR-UDC I-NK” or “FcR-UDC I/II-NK.”

### Example 2: NK Differentiation from Parental and Engineered Human Pluripotent Stem Cells

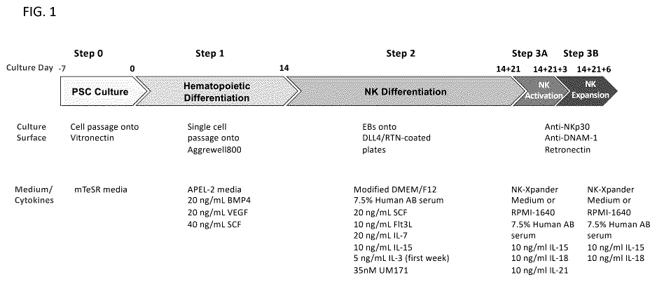

Parental and engineered UDC PSCs (collectively, “PSCs”) were thawed, expanded, and cultured in mTeSR™ (STEMCELL Technologies, Cambridge, MA) feeder-free, animal component-free media for up to seven days on vitronectin. The PSCs were dissociated into single cells and transferred into microwell plates and cultured in STEMdiff™ APEL™2 and Gibco® PFHM-II media (STEMCELL Technologies, Cambridge, MA; ThermoFisher Scientific, Waltham, MA) for fourteen days to differentiate into precursor cells (via embryoid bodies) capable of differentiating to hematopoietic cell types, endothelial cell types, and/or mesodermal derivatives. The media was supplemented with BMP4 (20 ng/ml), VEGF (20 ng/ml), and SCF (40 ng/ml). Half media changes were performed every other day starting on day 3. The embryoid bodies were replated on DLL4/Retronectin-coated T75 flasks to differentiate into NK cells. The embryoid bodies were cultured under feeder free conditions in differentiation media comprising SCF (20 ng/ml), IL-7 (20 ng/ml), IL-15 (10 ng/ml), Flt3L (10 ng/mL), IL-3 (5 mg/mL) and 35 nM UM171 for 21 days in modified DMEM/F12. Half media changes were performed twice a week. The resulting NK cells (“UDC-NK”) were washed and harvested. The UDC-NK cells were activated and expanded by culturing the UDC-NK cells in flasks coated with anti-NKp30 antibodies, anti-DNAM-1 antibodies, and Retronectin in CTS™ NK-Xpander™ or RPMI-1640 medium comprising IL-15 (10 ng/ml), IL-18 (10 ng/ml), and IL-21 (10 ng/ml) for three days, after which the UDC-NK cells were transferred to a G-REX® or spinner flask bioreactor and cultured in the presence of IL-15 and IL-18 for another three days. A representative schematic of the NK differentiation process is shown in FIG. 1.

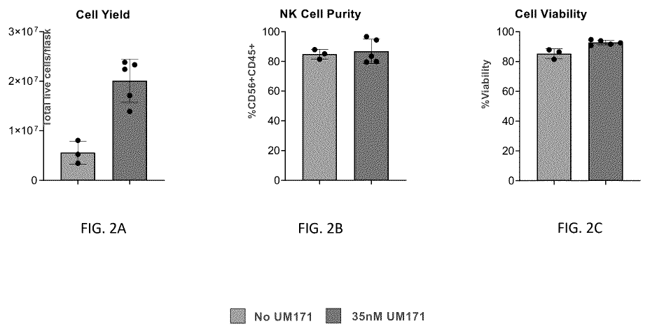

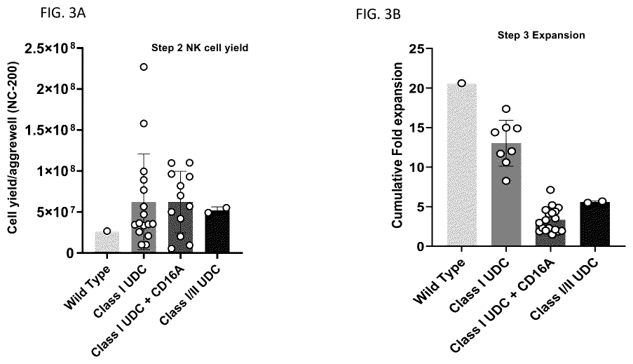

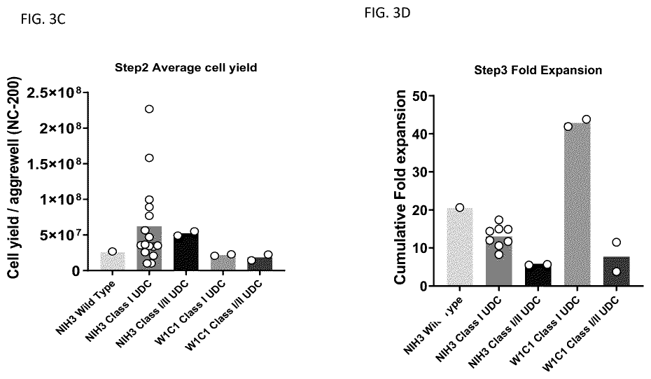

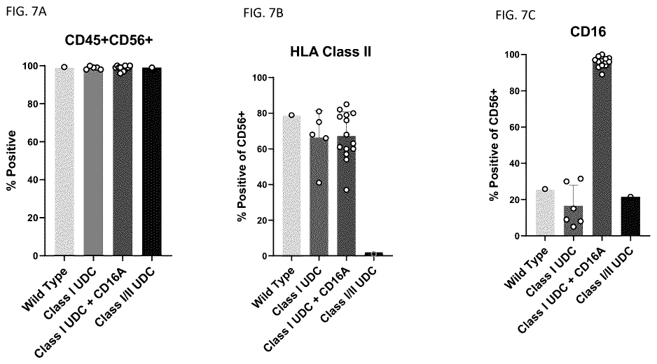

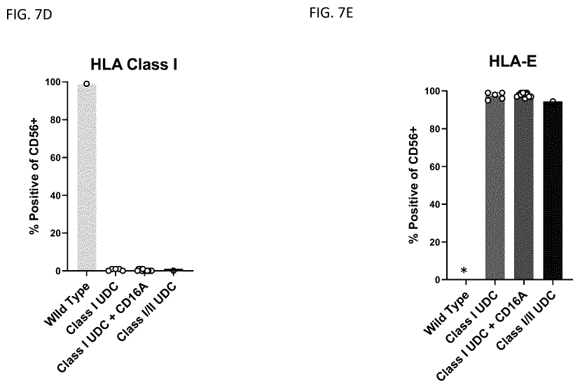

The method produced a substantially homogenous population of UDC NK cells regardless of parental cell line or engineered UDC PSC. Starting with about 4×10⁶ iPSC, at the beginning of Step 0 (as illustrated in FIG. 1), approximately 5×10¹⁰ of NK cells were produced, up to a 10,000-fold expansion. The addition of UM171 improved UDC-NK cell yield during NK differentiation as shown in FIGS. 2A-2C. Further, across multiple production runs and gene-edited clones, at least 90% of cells were CD56- and CD45-positive without requiring any purification step to remove undesired cell types (see FIG. 7A). Both UDC I-PSC and FR-UDC I-PSC produced at least 90% CD45+CD56+NK cells. In addition, as shown in FIGS. 3A & 3B, activation and expansion of NK cells resulted in consistent, high cell yield across PSC cell line (NIH3 and W1C1) and gene edited clones (UDC I-PSC, UDC I/II-PSC, and FcR-UDC I-PSC).

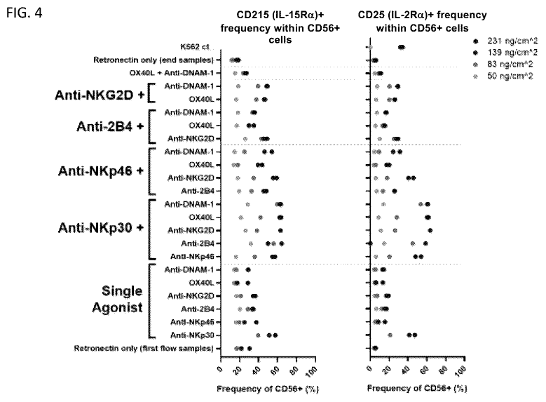

For the activation and expansion of the UDC-NK cell (Step 3 or step (d)), combinations of NK agonists were tested (see FIG. 4). Single NK agonists were also tested and showed moderate NK activation. The UDC-NK cells (Step 2) showed robust expansion and high cell viability. As shown in FIGS. 5A-5B, across four different cell seeding densities, the UDC NK cells (Step 2) cultured on Retronectin, anti-NKp30 antibodies, anti-DNAM-1 antibodies with media comprising IL-15, IL-18, and IL-21 demonstrated upwards of 16- to 32-fold expansion, as compared to Step 2 control which only had 3- to 4-fold expansion, and greater than 90% cell viability.

An analysis of the cell population subset and cellular impurities at the end of the NK differentiation process show very low levels of CD3+ T cells and little, if any, remaining PSCs. Table 1 provides the details of the cell population subsets/impurities.

TABLE 1

| | | % of Cells Expressing | | |
|---|---|---|---|---|
| Cell Phenotype | Cell Type | Mean ± s.d. | Range | Batches |
| CD45+ | Hematopoietic Cells | 99.5 ± 0.8 | 98.0-99.9 | 5 |
| CD45+ CD56+ | NK Cells | 96.7 ± 1.8 | 93.9-98.7 | 5 |
| CD45+ CD56− CD11b+ | Myeloid Cells | 2.00 ± 0.70 | 1.14-2.87 | 5 |
| CD45+ CD56− CD15+ | Granulocytes | 0.50 ± 1.06 | 0.007-2.40 | 5 |
| CD45+ CD56− CD33+ | Myeloid Lineage Cells | 0.12 ± 0.09 | 0.04-0.27 | 5 |
| CD45+ CD56− CD14+ | Monocytes | 0.02 ± 0.02 | 0.001-0.04 | 5 |
| CD45+ CD56− CD3+ | T Cells | 0.01 ± 0.01 | 0.001-0.030 | 5 |
| CD45+ CD56+ CD3+ | Possible NKT Cells | 0.14 ± 0.18 | ND-0.33 | 5 |
| CD45+ CD56− CD19+ | B Cells | 0.015 ± 0.010 | 0.001-0.030 | 5 |
| CD45+ CD56− CD34+ | Hematopoietic Stem and | 0.011 ± 0.013 | ND-0.030 | 5 |
| | Progenitor Cells | | | |
| SSEA3+ Tra-181+ | PSC | 0.0002 ± 0.0004 | ND-0.001 | 5 |

ND = not detected

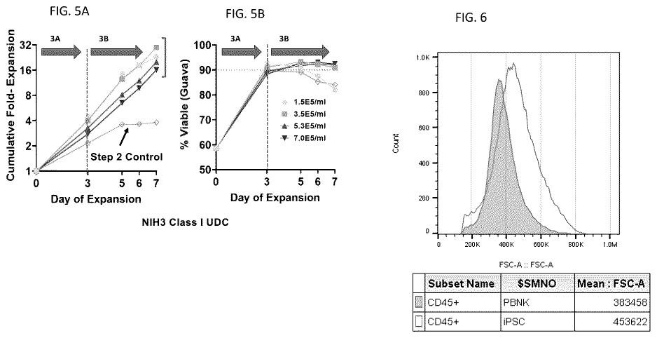

The UDC-NK cells of the presently disclosed subject matter were on average approximately 18% larger by volume compared to peripheral blood NK cells (PB-NK) as demonstrated by flow cytometry (see FIG. 6).

### Example 3: In Vitro Characterization of UDC PSC-Derived NK Cells

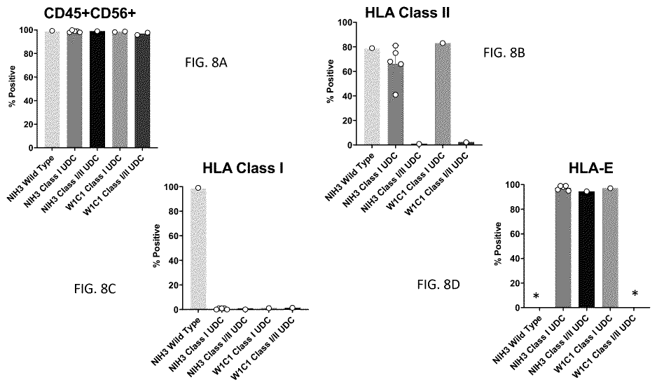

The UDC-NK cells derived from the UDC I-PSC, UDC I/II-PSC, and UDC I-FcR-PSC expressed the expected cell phenotype across different gene edited clones. As shown in FIG. 7A, the parental- and UDC PSC-derived UDC I-PSC-derived NK cells all had a high positivity for CD45+CD56+. UDC I-PSC-derived NK cells (UDC I-NK cells) failed to express HLA class I, but expressed HLA class II and HLA-E, and wild-type levels of CD16 (FcR). See FIGS. 7B-7E. FcR-UDC I-PSC-derived UDC-NK (FcR-UDC I-NK cells) cells had similar expression profile, except expressed higher levels of CD16 as compared to wild-type. Lastly, UDC I/II-PSC-derived UDC-NK cells (UDC I/II-NK cells) failed to express both HLA class I or class II protein, expressed HLA-E, and express wild-type levels of CD16. This data demonstrates that the NK cells derived from the gene edited clones maintained the gene edits after differentiation from the PSCs. The expected cell phenotype was also demonstrated across different PSC cell lines (NIH3 and W1C1) (see FIGS. 8A-8B). Based at least upon on the foregoing, the NK differentiation process disclosed herein is robust across different gene edited clones as well as PSC cell lines.

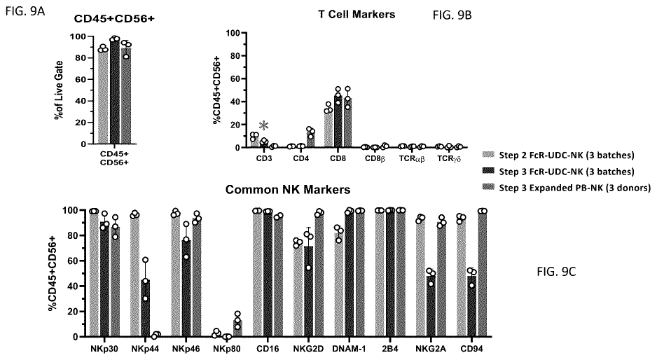

The UDC-NK cells expressed common NK markers and other biomarkers similar to PB-NK. As shown in FIG. 9C, common NK markers, e.g., NKp30, NKp46, NKG2D, DNAM-1, 2B4, NKG2A, and CD94, with some exceptions, were expressed on FcR-UDC I-NK cells (both Step 2 and Step 3 illustrated in FIG. 1, i.e., step (b) and (c), respectively) like expanded PB-NK cells (PB-NK cells were expanded with the Step 3 of the NK differentiation protocol). Similarly, T cell marker CD8 was commonly expressed in FcR-UDC I-NK cells and expanded PB-NK but not CD4, TCRαβ, or TCRγδ, and little to no CD3. FIG. 9B. The lack of TCRαβ plays an important role in safety.

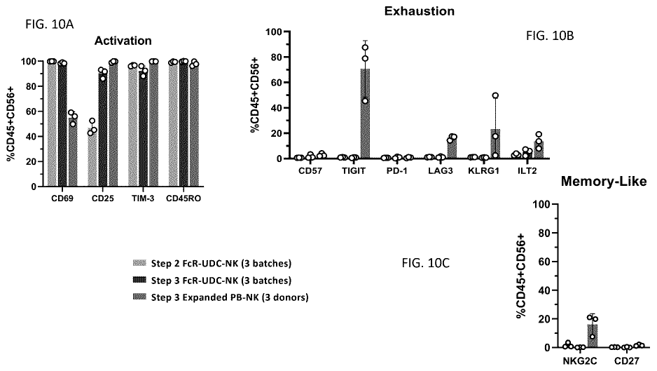

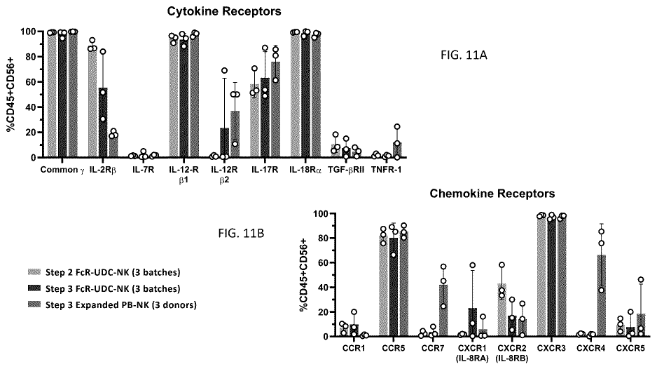

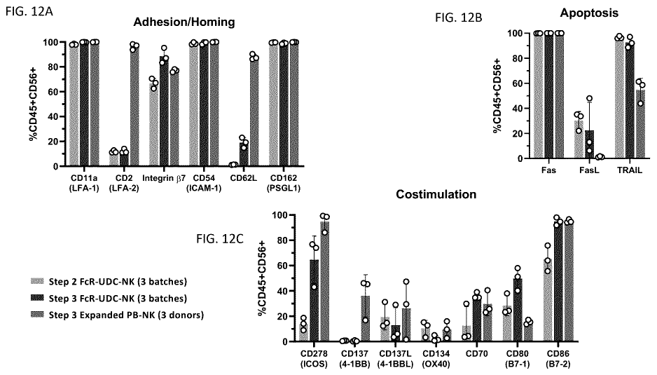

The UDC-NK cells expressed several activation markers and little, if any, exhaustion or “memory-like” markers. In FIGS. 10A-10C, FcR-UDC I-NK cells (Step 2 and Step 3) and expanded PB-NK were measured for activation, exhaustion, and “memory-like” markers. Apart from CD69, activation markers were, for the most part, similar between FcR-UDC I-NK cells and expanded PB-NK. FcR-UDC I-NK cells showed higher levels of CD69 compared to expanded PB-NK. Unlike expanded PB-NK, FcR-UDC I-NK cells showed little, if any, exhaustion or “memory-like” markers. Expanded PB-NK had higher levels of exhaustion markers TIGIT, LAG3, KLRG1 and ILT2, and higher levels of memory-like marker NKG2C. Differential expression of cytokine and chemokine receptors (see FIGS. 11A-11B) and adhesion/homing, costimulatory, and apoptotic markers (see FIGS. 12A-12C) are shown for FcR-UDC I-NK cells and expanded PB-NK.

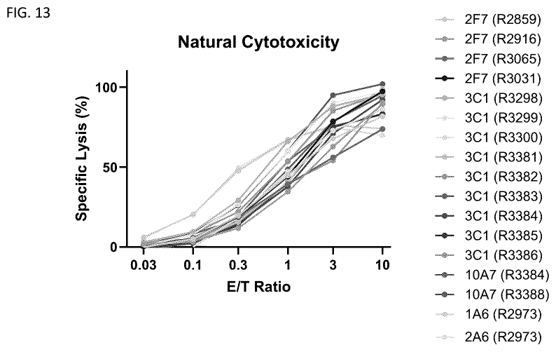

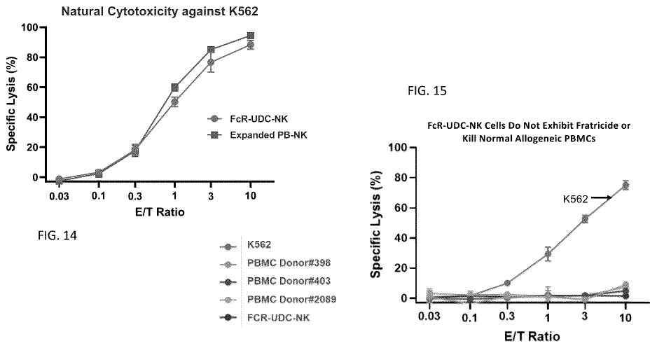

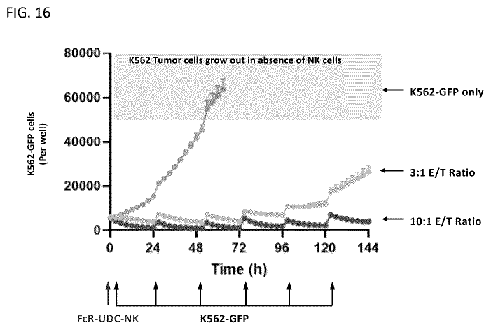

The UDC-NK cells also demonstrated natural cellular cytotoxicity (NCC) activity across multiple runs, gene edited clones, and PSC cell lines. Using a 2-hour europium release assay, NCC activity of UDC-NK cells was measured by their ability to kill K562 tumor target cells. In FIG. 13, 1×10⁴ K562-GFP tumor cells were added per well and a corresponding number of UDC-NK cells were added per the E:T ratio. The results demonstrate consistent and similar in vitro NCC activity (i.e., specific lysis) of the UDC-NK cells against K562 tumor cells regardless of PSC cell line or gene edited clones across multiple runs. Further, FcR-UDC I-NK cells exhibited similar NCC activity to expanded PB-NK; at the same time, no cytotoxicity nor fratricide was observed against normal allogeneic peripheral blood mononuclear cells (PBMC) or FcR-UDC I-NK cells themselves respectively (see FIG. 15). Surprisingly, these UDC-NK cells demonstrated serial NCC activity against K562 tumor cells such that the UDC-NK cells were capable of killing freshly added K562 tumor cells in a dose-dependent manner (see FIG. 16). IncuCyte serial killing assay was performed using 1.5×10⁴ (3:1 E:T) or 5×10⁴ (10:1 E:T) UDC-NK cells (FcR-UDC I-NK) (c.3C1). Every 24 hours, 5×10³ K562-GFP target cells added for a total of 6 times. Wells were imaged every 3 hours for 6 days.

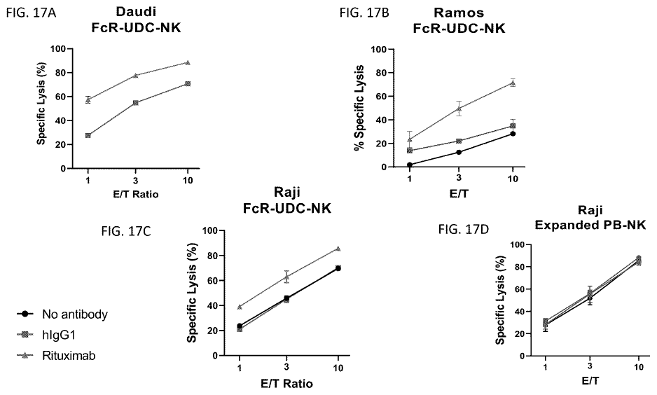

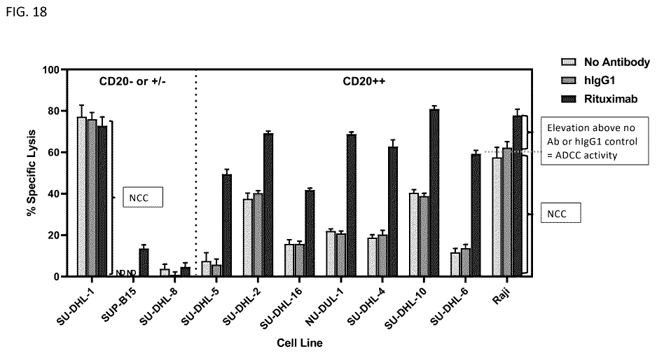

Similar to NCC, the UDC-NK cells exhibit potent antibody-dependent cellular cytotoxicity (ADCC) against a broad range of CD20+tumor cell lines. Using a 4-hour flow-based ADCC assay, tumor cells were labeled with cell trace violet and antibodies were dosed at 1 μg/ml. FcR-UDC I-NK cells showed an increase in ADCC activity in conjunction with Rituximab against three (3) tumor cell lines (Daudi, Ramos, and Raji) as compared to a control IgG1 antibody (see FIGS. 17A-17C). In contrast, expanded PB-NK cells plus Rituximab showed no increase in ADCC activity against Raji tumor cells as compared to the control IgG1 antibody (see FIG. 17D). Further, the FcR-UDC I-NK cells also demonstrated NCC and ADCC activity against a broad range of B lymphoma cell lines (see FIG. 18). These results demonstrate ADCC activity of UDC-NK cells against a broad range of CD20+ and B lymphoma cell lines.

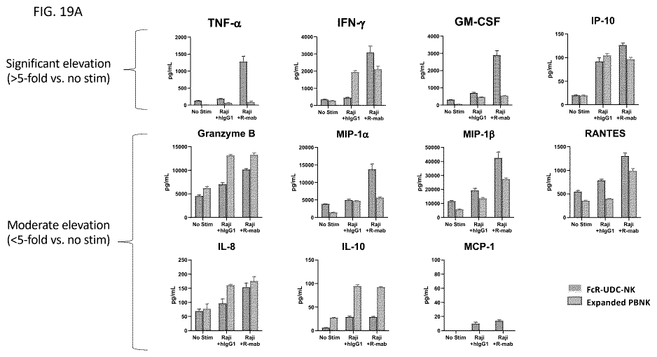

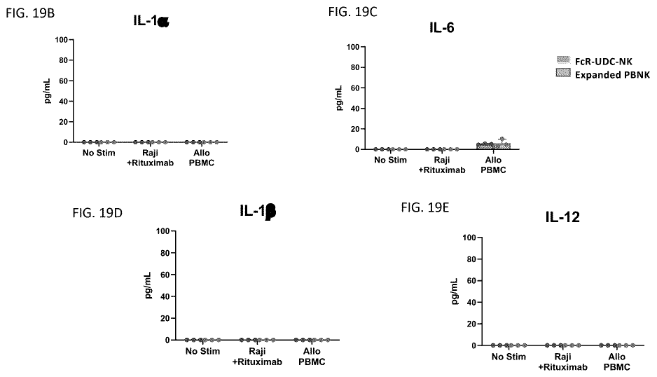

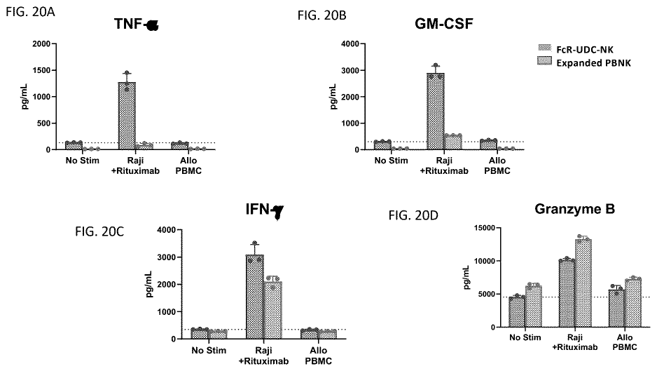

The UDC-NK cells produced cytokine profiles indicative of active NK cells capable of inducing ADCC after stimulation with Rituximab-coated Raji tumor cells. Using a standard cytokine secretion assay, 1×10⁵ FcR-UDC I-NK cells/well were co-cultured with Rituximab-coated Raji tumor cells at a 1:1 E:T ratio for 25 hours. Cytokine secretion was measured using a 15-Plex Cytokine Luminex immunoassay (see FIG. 19A). Moreover, FcR-UDC I-NK cells did not elevate cytokines/chemokines secretion following co-culture with allogeneic PBMCs demonstrating specificity of the FcR-UDC I-NK cells (see FIGS. 19B-E and 20A-D).

### Example 4: In Vivo UDC-PSC-Derived NK Cells Persistence and Distribution

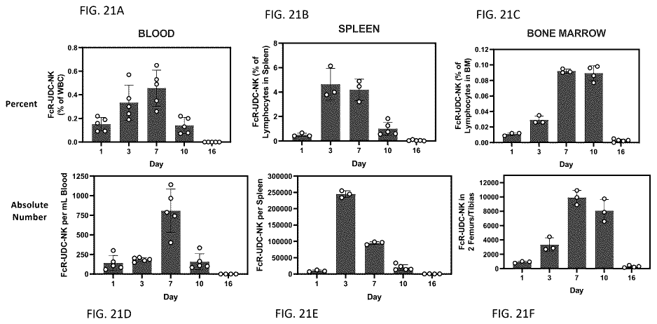

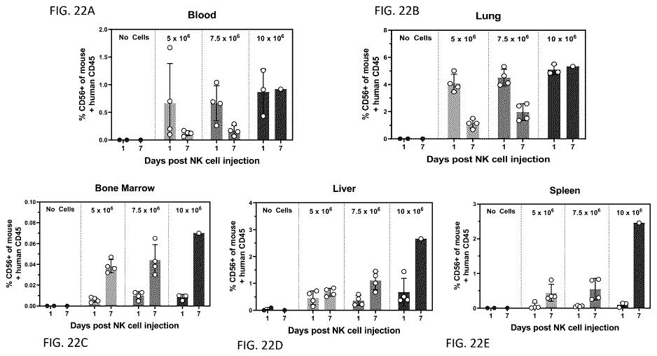

The UDC-NK cells also exhibited persistence in vivo in the blood, spleen, and bone marrow in the absence of antigen. In NSG mice, 4×10⁶ FcR-UDC I-NK cells (c.3C1) were injected intravenously (i.v.) followed by IL-2 (30,000 IU, 3×/wk.) and IL-15 (100 ng, daily for 7 days). For each tissue, the percentage of FcR-UDC I-NK cells within lymphocytes/WBC was determined by flow cytometry and staining for human CD45 and human CD56. Total cell counts were determined using a flow cytometric method (per mL blood, per spleen, per BM cells in 2 femurs+2 tibia), and the % FCR-UDC I-NK was applied to this number to calculate the number of FcR-UDC I-NK in each tissue. As shown in FIGS. 21A-21F, FcR-UDC I-NK cells persisted in vivo in the absence of antigen for greater than 10 days post i.v. injection. Further, FcR-UDC I-NK cells distributed to multiple tissues: lung>liver>spleen>blood>bone marrow (see FIGS. 22A-22E).

### Example 5: In Vivo Anti-Tumor Efficacy Studies Using UDC-NK Cells in a Raji i.p. Tumor Model

The efficacy of the UDC-NK cells against tumors in vivo was studied in a Raji intraperitoneal (i.p.) tumor model. In a representative study, NSG mice were divided into six group (n=7 or 8) as shown in Table 2 below.

TABLE 2

| Group No. | Treatment |
|---|---|
| 1 | Untreated |
| 2 | Rituximab alone |
| 3 | FcR-UDC-NK cells alone |
| 4 | Expanded PB-NK alone |
| 5 | FcR-UDC-NK cells + Rituximab |
| 6 | Expanded PB-NK + Rituximab |

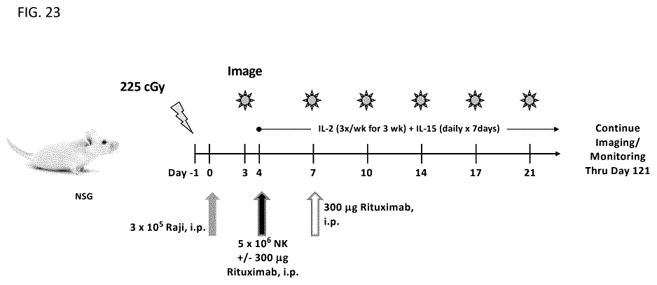

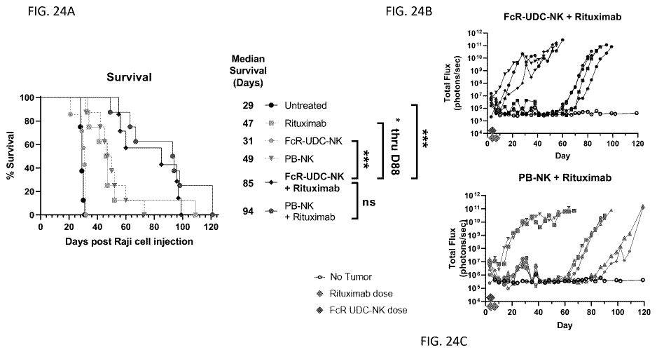

In FIG. 23, NSG mice were irradiated and injected i.p. with Raji (Burkitt's lymphoma) cells (3×10⁵ cells). Raji cells are resistant to NK cells in the absence of an anti-CD20 antibody. At day four, 5×10⁶ FcR-UDC I-NK cells and/or Rituximab (300 μg) was administered to the NSG mice. At day 7, a second dose of Rituximab (300 μg) was administered. NSG mice were administered daily doses of IL-15 (100 ng) for 7 days, and IL-2 (30,000 IU) was administered 3×/week for 3 weeks. The NSG mice were imaged and assessed for survival, BLI (tumor burden), and body weight and other health conditions at various days through day 121. As shown in FIGS. 24A-24B, the combination of FcR-UDC I-NK and Rituximab significantly prolonged survival of lymphoma-bearing NSG mice, beyond that observed using Rituximab or FcR-UDC I-NK cells alone (see FIG. 24A). The efficacy was similar between FcR-UDC I-NK cells plus Rituximab and PB-NK cells plus Rituximab (see FIG. 24B). However, the responder rate was approximately 50%.

In another study, mice were divided into five group (n=8) as shown in Table 3 below.

TABLE 3

| Group No. | Treatment |
|---|---|
| 1 | Untreated |
| 2 | Rituximab alone (Day 2) |
| 3 | FcR-UDC-NK cells alone (Day 2, 6, and 10) |
| 4 | FcR-UDC-NK cells + Rituximab (Day 2) and FcR- |
| | UDC-NK cells alone (Day 6 and 10) |
| 5 | FcR-UDC-NK cells + Rituximab (Day 2) + FcR-UDC- |
| | NK cells alone (Day 10 and 16) |

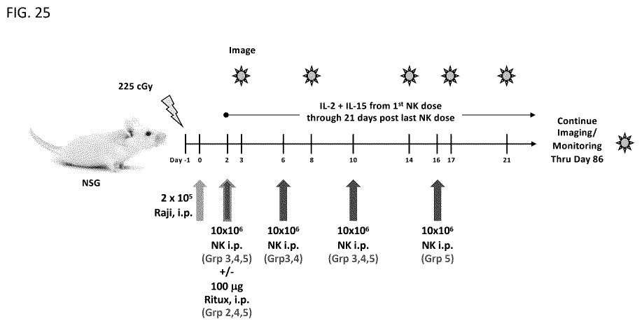

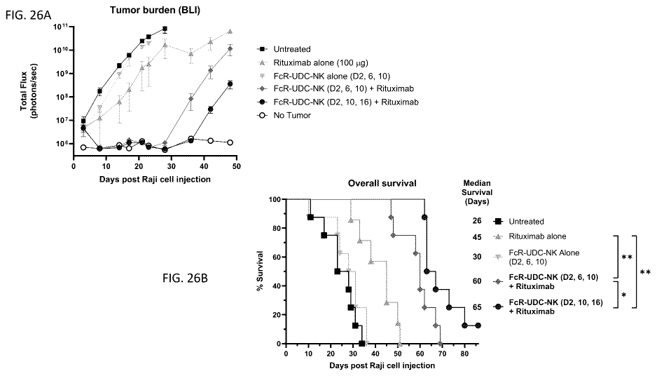

In FIG. 25, NSG mice were irradiated and injected i.p. with Raji (Burkitt's lymphoma) cells (2×10⁵ cells). At day 2, 10×10⁶ FcR-UDC I-NK cells and/or 100 μg Rituximab was administered to the mice. At day 6, 10, and/or 16, 10×10⁶ FcR-UDC I-NK cells were administered i.p. to the mice. Mice were administered daily doses of IL-15 (100 ng) and IL-2 (30,000 IU) 3×/week from day 2 through 21 days post last NK dose. The NSG mice were imaged and assessed for survival, BLI (tumor burden), and body weight and other health conditions at various days through day 86. FcR-UDC I-NK and Rituximab significantly prolonged survival of lymphoma-bearing mice, beyond that observed using Rituximab or FcR-UDC-NK cells alone (see FIG. 26B). All mice treated with FcR-UDC-NK plus Rituximab responded to treatment and increasing the interval between doses prolonged the duration of the response (see FIG. 26A).

### Example 6: In Vivo Anti-Tumor Efficacy Studies Using UDC-NK Cells in a Raji i.v. Tumor Model

The efficacy of the UDC-NK cells against tumors in vivo was studied in a Raji i.v. tumor model. The Raji i.v. model is a more aggressive, disseminated model and represents a more clinically relevant, physiological model of lymphoma. In a representative study using NOGf mice (FcResolv NOG) that have mouse FcgR knocked out, NOGf mice were divided into three groups as shown in Table 4 below.

TABLE 4

| Group No. | Treatment | No. of Mice |
|---|---|---|
| 1 | Untreated | 8 |
| 2 | Rituximab alone (Day 1, 8, 15) | 8 |
| 3 | FcR-UDC-NK cells + Rituximab | 8 |
| | (Days 1, 8, and 15) and FcR-UDC- | |
| | NK cells alone (Day 4) | |

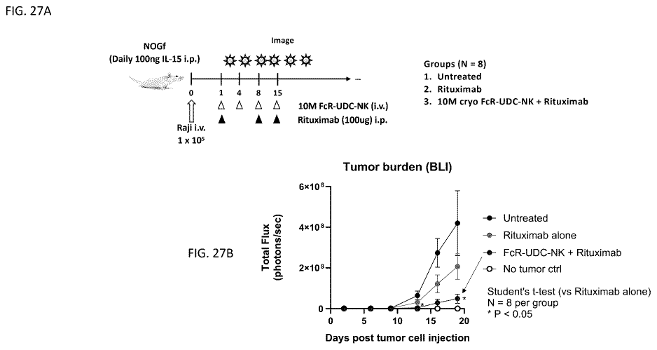

In FIG. 27A, NOGf mice were injected i.v. with Raji cells (1×10⁵ cells), at Day 1 with Rituximab (100 pug, i.p.) or in combination with FcR-UDC I-NK cells (10×10⁶, i.v.). At Day 8 and 15 Rituximab alone (100 μg, i.p.) or in combination with FcR-UDC I-NK cells (10×10⁶, i.v.) were administered to the mice. For group 3, FcR-UDC I-NK cells alone (10×10⁶, i.v.) were administered to the mice on day 4. Mice were administered daily doses of IL-15 (100 ng, i.p.). As shown in FIG. 27B, the combination of FcR-UDC I-NK plus Rituximab achieved a significantly greater reduction of tumor burden compared to Rituximab alone as well as untreated.

In another study, mice were divided into four group (n=8) as shown in Table 5 below.

TABLE 5

| Group No. | Treatment | No. of Mice |
|---|---|---|
| 1 | Untreated | 8 |
| 2 | Rituximab alone (Days 1, 8, 11) | 8 |
| 3 | 3M FcR-UDC I-NK cells + Rituximab | 8 |
| | (Days 1, 8, and 11) and 3M FcR- | |
| | UDCI-NK cells alone (Day 4) | |
| 4 | 15M FcR-UDC I-NK cells + Rituximab | 8 |
| | (Days 1 and 11), 15M FcR-UDC I-NK | |
| | cells alone (Day 4), and Rituximab | |
| | alone (Day 8) | |

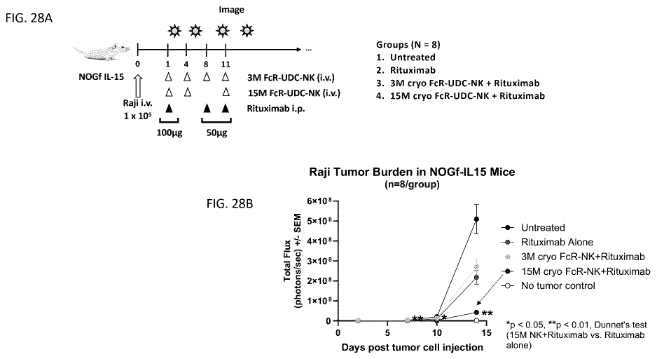

In FIG. 28A, NOGf mice engineered to express human IL-15 were injected i.v. with Raji cells (1×10⁵ cells), at Day 1 with Rituximab alone (100 μg, i.p.) or in combination with FcR-UDC I-NK cells (3×10⁶ or 15×10⁶). At Day 4, FcR-UDC I-NK cells were given alone (3×10⁶ or 15×10⁶, i.v.). At Day 8 and 11, Rituximab alone (50 μg, i.p.) was administered or was administered in combination with 3×10⁶ FcR-UDC I-NK cells (i.v.), or on Day 11 Rituximab was administered in combination with 15×10⁶ FcR-UDC I-NK cells (i.v.). As shown in FIG. 28B, the combination of 15×10⁶ FcR-UDC I-NK cells+Rituximab achieved the greatest reduction in tumor burden.

### Example 7: Anti-Tumor Efficacy Studies Using UDC-NK Cells and an Anti-Claudin 6 (CLDN6) Antibody

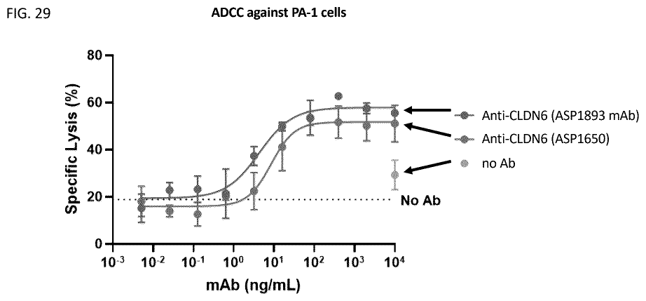

Antibody-dependent cell cytotoxicity (ADCC) was evaluated using the UDC-NK cells described herein in combination with anti-CLDN6 antibodies against the CLDN6+ovarian cancer cell line PA-1. Briefly, in a four-hour flow-based ADCC assay, CellTrace Violet-labeled PA-1 cells were co-cultured with FcR-UDC I-NK cells in the presence of titrated concentrations of anti-CLDN6 antibodies, ASP1650 (also known as IMAB027) and ASP1893 mAb. As shown in FIG. 29, the FcR-UDC I-NK cells demonstrated enhanced cytotoxicity against PA-1 cells in an anti-CLDN6 antibody dose-dependent manner. The EC50 using antibody ASP1893 mAb (humanized IgG1) was about 4.32 ng/ml, and the EC50 using antibody ASP1650 (chimeric IgG1) was about 8.49 ng/ml.

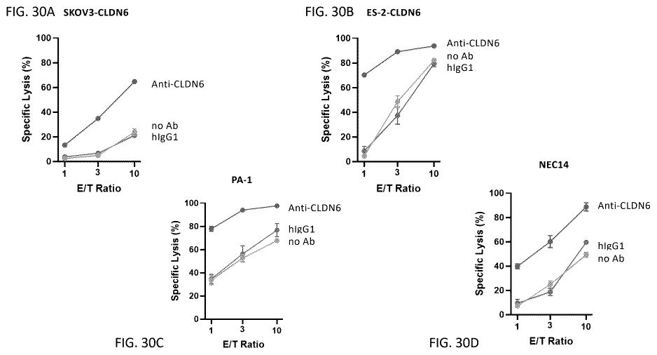

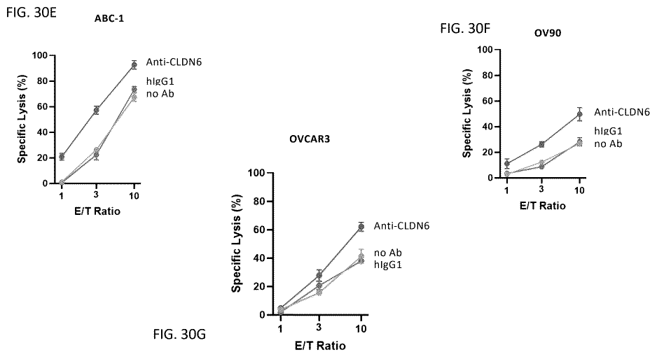

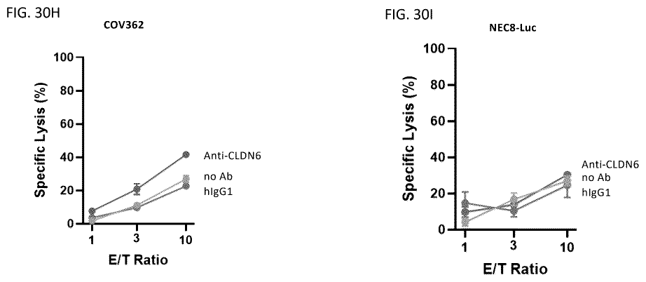

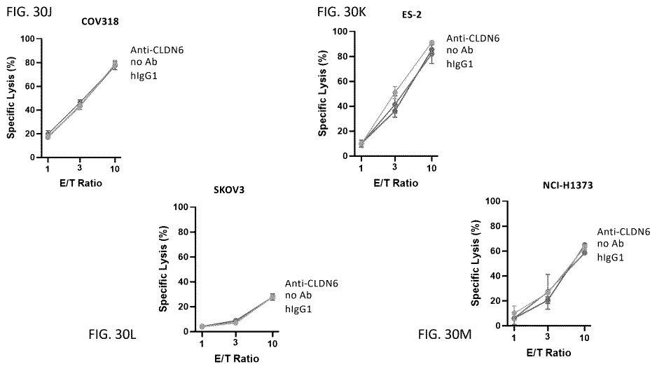

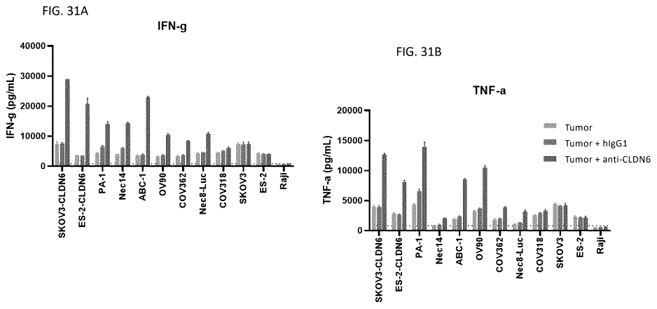

A tumor cell line panel was also tested for surface CLDN6 expression and categorized into CLDN6 hi, CLDN6mid, CLDN6low, and CLDN6-groups. The FcR-UDC I-NK cells were tested in a four-hour flow-based ADCC assay against a panel of tumor cell lines with variable levels of surface CLDN6. As shown in FIGS. 30A-30M, FcR-UDC I-NK cells demonstrated ADCC activity against CLDN6+tumor cells in a CLDN6 expression-dependent manner. Interestingly, FcR-UDC I-NK cells showed natural cytotoxicity (NCC) without anti-CLDN6 antibodies against a broad range of tumor cell lines, regardless of CLDN6 surface expression.

TNF-α and IFN-γ production by UDC-NK cells after stimulation with anti-CLDN6 antibody-coated tumor cells also was examined. Briefly, 105 FcR-UDC I-NK cells and 105 tumor cells were co-cultured in a 96-well plate in the presence or absence of 1 g/ml of anti-CLDN6 antibody (ASP1893 mAb) or control hIgG1 antibody. 24 hours after co-culture, culture supernatants were harvested for TNF-α and IFN-γ measurements by ELISA (R&D Systems). FIGS. 31A-31B show that FcR-UDC I-NK cells exhibited elevated TNF-α and IFN-γ secretions after stimulation with a panel of tumor cell lines compared to the basal cytokine levels without stimulation. FcR-UDC I-NK cells demonstrated superior cytokine production upon stimulation with CLDN6+tumor cells and ASP1893 mAb.

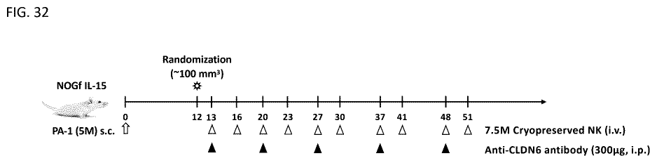

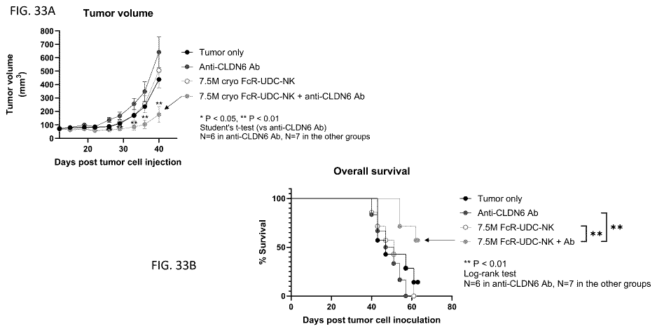

The efficacy of the UDC-NK cells against tumors in vivo was studied in a CLDN6+PA-1 cell xenograft mouse tumor model. In a representative study, mice were divided into four group (n=6 or 7) as shown in Table 6 below. Briefly, 5×10⁶ PA-1 cells were engrafted into the flank of NOGf mice engineered to express human IL-15 subcutaneously. 13 days after tumor engraftment, mice were treated with 7.5×10⁶ cryopreserved FcR-UDC I-NK cells i.v. with or without anti-CLDN6 (300 μg ASP1650) i.p. as illustrated in FIG. 32. Cryopreserved FcR-UDC-I-NK cells were thawed and immediately injected to mice. FIGS. 33A-33B show that combination treatment with FcR-UDC I-NK cells and anti-CLDN6 antibody demonstrated statistically significant anti-tumor efficacy and improved overall survival. Monotherapy of FcR-UDC I-NK cells or anti-CLDN6 antibody showed no significant anti-tumor efficacy.

TABLE 6

| Group No. | Treatment | No. of mice |
|---|---|---|
| 1 | Untreated | 7 |
| 2 | Anti-CLDN6 Ab alone | 6 |
| 3 | 7.5M UDC-NK | 7 |
| 4 | 7.5M UDC-NK + anti-CLDN6 Ab | 7 |

The above example demonstrates that UDC-NK cells exhibit natural cytotoxicity in vitro without anti-CLDN6 antibodies against a broad range of tumor cell lines, regardless of CLDN6 surface expression. The example further demonstrates that UDC-NK cells of the disclosure exhibit potent anti-tumor activity in vitro and in vivo when used in conjunction with an anti-CLDN6 antibody.

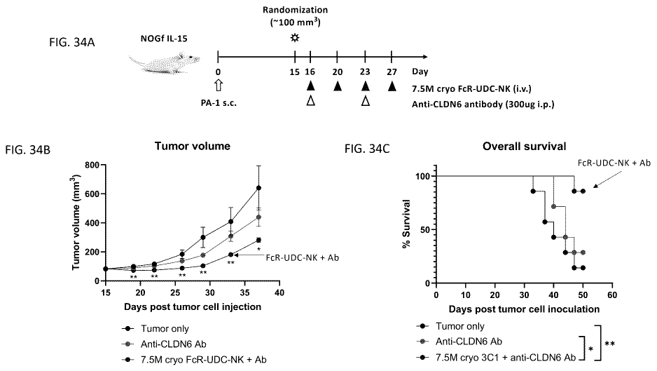

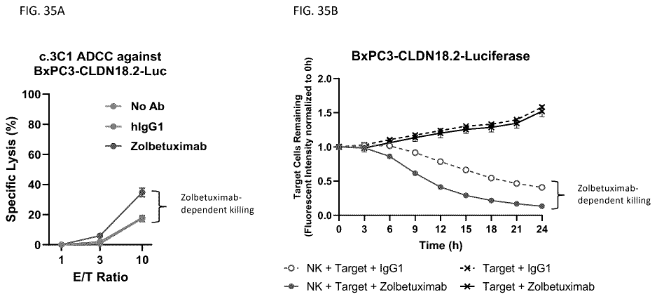

In another study, a PA-1 cell xenograft mouse tumor model using NOGf mice expressing human IL-15 was employed. As illustrated in FIG. 34A, 7.5×10⁶ FcR-UDC I-NK cells were administered i.v. and/or anti-CLDN6 antibody (300 μg) was administered i.p. The combination treatment of FcR-UDC-NK cells and anti-CLDN6 antibody resulted in tumor growth inhibition (FIG. 34B) and prolonged the survival (FIG. 34C). Greater than 50% survival rate was observed at about 50 days in mice administered FcR-UDC 1-NK cells+anti-CLDN6 antibody.

### Example 8: Anti-Tumor Studies Using UDC-NK Cells and an Anti-Claudin 18.2 (CLDN18.2) Antibody

This example demonstrates that UDC-NK cells demonstrate anti-tumor activity when used in combination with an anti-CLDN18.2 antibody, Zolbetuximab. Briefly, ADCC assays were performed as a four-hour co-culture with FcR-UDC I-NK cells and BxPC3-CLDN18.2-Luc tumor target cells, similar to the assay described in Example 7. Zolbetuximab was present at 1 g/mL. The results are illustrated in FIG. 35A, which shows Zolbetuximab-dependent killing of CLDN18.2-expressing tumor cells. The anti-tumor effect of FcR-UDC I/II-NK cells in combination with anti-claudin 18.2 antibody (Zolbetuximab) against BxPC3-CLDN18.2-Luciferase tumor cells is illustrated in FIG. 35B, which demonstrates that the highest level of cell killing was achieved using FcR-UDC I/II-NK cells in combination with Zolbetuximab.

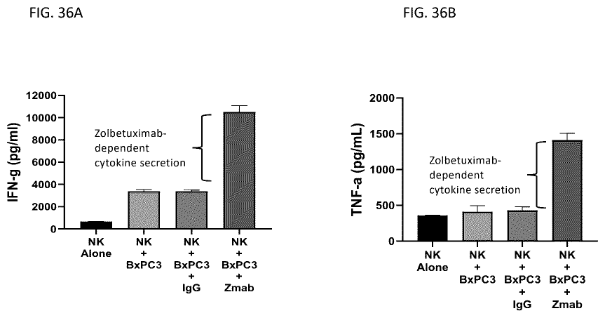

A cytokine secretion assay also was performed using methods similar to those in Example 7. BxPC3-CLDN18.2-Luc cells were cultured with FcR-UDC I-NK cells of the disclosure, FcR-UDC I-NK cells and human lgG, or FcR-UDC I-NK cells and Zolbetuximab. Supernatants were collected after a 24-hour co-culture in the presence of IL-15 (10 ng/ml), and the level of interferon-gamma or tumor necrosis factor-alpha was examined. Zolbetuximab was present at 1 μg/mL. The results are illustrated in FIGS. 36A-36B. Cytokine secretion by FcR-UDC I-NK cells was greatly increased in samples comprising Zolbetuximab and BxPC3-CLDN18.2-Luc tumor target cells.

The examples provided herein establish that the engineered NK cells of the disclosure exhibit anti-tumor activity against a broad range of tumor types, including lymphoma, ovarian cancer, and pancreatic cancer. The examples report that NK cells exhibit ant-tumor activity alone, and anti-tumor activity is potentiated by use of antibody constructs against tumor antigens (such as CLDN6, CLDN18.2, and CD20). While the working examples utilized tumor cells to demonstrate the cell-killing activity of the engineered NK cells of the disclosure, it will be appreciated that the data may be extrapolated to additional cell types.

All references, including publications, patent applications, and patents, cited herein are hereby incorporated by reference to the same extent as if each reference were individually and specifically indicated to be incorporated by reference and were set forth in its entirety herein.

What is claimed:

1. A population of engineered natural killer (NK) cells, wherein the NK cells express at least one polypeptide selected from the group consisting of human leukocyte antigen E (HLA-E), human leukocyte antigen F (HLA-F), and human leukocyte antigen G (HLA-G);
wherein the NK cells comprise a genetically engineered disruption of one or more copies of endogenous β-2 microglobulin (B2M); and
wherein the NK cells comprise a genetically engineered disruption of one or more copies of a human leukocyte antigen (HLA) class II-related gene selected from the group consisting of regulatory factor X associated ankyrin containing protein (RFXANK), regulatory factor 5 (RFX5), regulatory factor X associated protein (RFXAP), and class II transactivator (CIITA).

2. The population of engineered NK cells of claim 1, wherein the NK cells further comprise a nucleic acid molecule encoding a suicide gene product.

3. The population of engineered NK cells of claim 2, wherein NK cells comprise a herpes simplex virus thymidine kinase (TK) suicide gene.

4. The population of engineered NK cells of any one of claims 1-3, wherein the NK cells are derived from pluripotent stem cells.

5. The population of engineered NK cells of claim 4, wherein the pluripotent stem cells are embryonic stem cells.

6. The population of engineered NK cells of claim 4, wherein the pluripotent stem cells are induced pluripotent stem cells.

7. The population of engineered NK cells of any one of claims 1-6, wherein the NK cells express one or more cell surface markers selected from the group consisting of CD56, CD45, CD25, DNAM-1, NKp30, NKG2D, and NKp44.

8. The population of engineered NK cells of any one of claims 1-7, wherein the population of engineered NK cells is capable of engrafting into a subject following administration.

9. The population of engineered NK cells of claim 8, wherein at least 50% of the population of engineered NK cells is capable of engrafting into a target tissue of the subject.

10. The population of engineered NK cells of claim 8 or claim 9, wherein the population of engineered NK cells is capable of remaining engrafted in the target tissue of the subject for at least 20 days following administration.

11. The population of engineered NK cells of any one of claims 8-10, wherein the target tissue is blood or bone marrow.

12. The population of engineered NK cells of any one of claims 1-11, wherein the NK cells are capable of inducing antibody-dependent cell-mediated cytotoxicity (ADCC) of a cancer cell.

13. The population of engineered NK cells of any one of claims 1-12, wherein the NK cells are capable of inducing natural cell cytotoxicity (NCC) of a cancer cell.

14. The population of engineered NK cells of claim 12 or claim 13, wherein the NK cells decrease tumor load by at least 10%.

15. The population of engineered NK cells of any one of claims 1-14, wherein the NK cells have at least about a 5% increase in cell volume as compared to peripheral blood NK cells.

16. The population of engineered NK cells of any one of claims 1-15, wherein the cells express a chimeric antigen receptor or an Fc receptor.

17. The population of engineered NK cells of any one of claims 1-16, wherein the NK cells comprise a genetically engineered disruption of all copies of endogenous B2M.

18. The population of engineered NK cells of any one of claims 1-17, wherein the NK cells comprise a genetically engineered disruption of all copies of an HLA class II-related gene selected from the group consisting of RFXANK, RFX5, RFXAP, and CIITA.

19. A method of producing human pluripotent stem cell-derived engineered natural killer (NK) cells under feeder free conditions, the method comprising
(a) culturing human pluripotent stem cells genetically engineered to disrupt one or more copies of endogenous β-2 microglobulin (B2M) and one or more copies of a human leukocyte antigen (HLA) class II-related gene selected from the group consisting of regulatory factor X associated ankyrin containing protein (RFXANK), regulatory factor 5 (RFX5), regulatory factor X associated protein (RFXAP), and class II transactivator (CIITA);
(b) differentiating the human pluripotent stem cells to precursor cells capable of differentiating to hematopoietic cell types, endothelial cell types, and/or mesodermal derivatives;
(c) differentiating the precursor cells to NK cells under feeder free conditions in a culture media comprising SCF, IL-7, IL-15, and FIt3L; and
(d) culturing the NK cells in a culture media comprising IL-15 and IL-18, and in the presence of an antibody or ligand that binds a receptor on NK cells to promote activation of the NK cells,
thereby generating human pluripotent stem cell-derived engineered NK cells.

20. A method of producing human pluripotent stem cell-derived engineered natural killer (NK) cells under feeder free conditions, the method comprising
(a) culturing human pluripotent stem cells genetically engineered to disrupt one or more copies of endogenous β-2 microglobulin (B2M) and one or more copies of a human leukocyte antigen (HLA) class II-related gene selected from the group consisting of regulatory factor X associated ankyrin containing protein (RFXANK), regulatory factor 5 (RFX5), regulatory factor X associated protein (RFXAP), and class II transactivator (CIITA);
(b) differentiating the human pluripotent stem cells using embryoid bodies to precursor cells capable of differentiating to hematopoietic cell types, endothelial cell types, and/or mesodermal derivatives; and
(c) differentiating the precursor cells to NK cells under feeder free conditions in a culture media comprising SCF, IL-7, IL-15, and FIt3L;
thereby generating human pluripotent stem cell-derived engineered NK cells.

21. The method of claim 20, further comprising (d) culturing the NK cells in a culture media comprising IL-15 and IL-18, and in the presence of an antibody or ligand that binds a receptor on an NK cell to promote activation of the NK cells.

22. The method of claim 19 or claim 21, wherein step (d) comprises culturing the NK cells in the presence of an antibody that binds to DNAM-1, an antibody that binds to OX40, an antibody that binds to NKG2D, an antibody that binds to 2B4, an antibody that binds to NKp30, or an antibody that binds to NKp46, or a combination thereof.

23. The method of claim 22, wherein step (d) comprises culturing the NK cells in the presence of an antibody that binds NKp30 and an antibody that binds DNAM-1.

24. The method of claim 19 or claim 21, wherein step (d) comprises culturing the NK cells in the presence of a ligand that binds to DNAM-1, a ligand that binds to OX40, a ligand that binds to NKG2D, a ligand that binds to 2B4, a ligand that binds to NKp30, or a ligand that binds to NKp46, or a combination thereof.

25. The method of claim 24, wherein step (d) comprises culturing the NK cells in the presence of a ligand that binds NKp30 and a ligand that binds DNAM-1.

26. The method of any one of claim 19 or 21-25, wherein the culture media in step (d) further comprises IL-21.

27. The method of any one of claims 19-26, wherein the human pluripotent stem cells of step (a) further comprise a nucleic acid molecule encoding a suicide gene product.

28. The method of claim 27, wherein the NK cells comprise a herpes simplex virus thymidine kinase (TK) suicide gene.

29. The method of any one of claims 19-28, wherein the human pluripotent stem cells are human embryonic stem cells.

30. The method of any one of claims 19-28, wherein the human pluripotent stem cells are human induced pluripotent stem cells

31. The method of any one of claims 19-30, wherein the culture media of step (c) further comprises UM171.

32. The method of any one of claims 19-31, wherein the NK cells express one or more cell surface markers selected from the group consisting of CD56, CD45, CD25, DNAM-1, NKp30, NKG2D, and NKp44.

33. The method of any one of claims 19-32, wherein the NK cells express at least one polypeptide selected from the group consisting of human leukocyte antigen E (HLA-E), human leukocyte antigen F (HLA-F), and human leukocyte antigen G (HLA-G).

34. The method of any one of claims 19-33, wherein the NK cells are capable of engrafting into a subject following administration.

35. The method of claim 34, wherein at least 50% of the NK cells are capable of engrafting into the subject.

36. The method of claim 34 or claim 35, wherein the NK cells are capable of remaining engrafted in the subject for at least 20 days following administration.

37. The method of any one of claims 34-36, wherein the NK cells are capable of engrafting into the blood and bone marrow.

38. The method of any one of claims 19-37, wherein the NK cells are capable of inducing antibody-dependent cell-mediated cytotoxicity (ADCC) of a cancer cell.

39. The method of any one of claims 19-38, wherein the NK cells are capable of inducing natural cell cytotoxicity (NCC) of a cancer cell.

40. The method of claim 38 or claim 39, wherein the NK cells decrease tumor load by at least 10%.

41. The method of any one of claims 19 and 21-40, wherein step (d) is repeated up to five times.

42. A method of increasing the purity of a population of NK cells derived from pluripotent stem cells, wherein the method comprises differentiating embryoid bodies to NK cells under feeder free conditions in a culture media comprising SCF, IL-7, IL-15, and FIt3L and in the presence of UM171.

43. The method of claim 42, wherein the culture media further comprises IL-3.

44. The method of claim 42 or claim 43, wherein the method is performed in a vessel coated with a recombinant human fibronectin fragment and DLL4.

45. The method of any one of claims 42-44, wherein the method is performed for about one day to about 21 days.

46. A pharmaceutical composition comprising the population of engineered NK cells of any one of claims 1-18, and a pharmaceutically acceptable carrier.

47. A method of treating a disease or disorder in a subject in need thereof, the method comprising administering to the subject an effective amount of the population of engineered NK cells of any one of claims 1-18, or the pharmaceutical composition of claim 46, thereby treating the disease or disorder in the subject.

48. The method of claim 47, wherein the disease or disorder is cancer.

49. The method of claim 47 or claim 48, wherein the method further comprises administering one or more cytokines to the subject.

50. The method of claim 49, wherein the one or more cytokines are IL-15 and/or IL-2.

51. The method of any one of claims 47-50, wherein the administering increases survival time of the subject by at least 20 days relative to survival time of the subject in the absence of administering the population of engineered NK cells.

52. The method of any one of claims 47-50, wherein the administering decreases tumor load in the subject.

53. The method of any one of claims 47-52, wherein the method further comprises administering one or more antibodies to the subject.

54. The method of claim 47, wherein the disease or disorder is an autoimmune disorder.

55. The method of claim 54, wherein the method further comprises administering one or more antibodies to the subject.


SEQUENCE LISTING (20 sequences)

SEQ ID NO: 1 - DNA, 987 bp, Homo sapiens
  source 1..987: mol_type = other DNA; note = B2M
  CDS 61..420: protein_id = 2; translation = MSRSVALAVLALLSLSGLEAIQRTPKIQVYSRHPAENGKSNFLN CYVSGFHPSDIEVDLLKNGERIEKVEHSDLSFSKDWSFYLLYYTEFTPTEKDEYACRV NHVTLSQPKIVKWDRDM
aatataagtggaggcgtcgcgctggcgggcattcctgaagctgacagcattcgggccgagatgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaagcagcatcatggaggtttgaagatgccgcatttggattggatgaattccaaattctgcttgcttgctttttaatattgatatgcttatacacttacactttatgcacaaaatgtagggttataataatgttaacatggacatgatcttctttataattctactttgagtgctgtctccatgtttgatgtatctgagcaggttgctccacaggtagctctaggagggctggcaacttagaggtggggagcagagaattctcttatccaacatcaacatcttggtcagatttgaactcttcaatctcttgcactcaaagcttgttaagatagttaagcgtgcataagttaacttccaatttacatactctgcttagaatttgggggaaaatttagaaatataattgacaggattattggaaatttgttataatgaatgaaacattttgtcatataagattcatatttacttcttatacatttgataaagtaaggcatggttgtggttaatctggtttatttttgttccacaagttaaataaatcataaaacttgatgtgttatctctta

SEQ ID NO: 2 - protein, 119 aa, Homo sapiens
  source 1..119: note = B2M
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 3 - DNA, 1369 bp, Homo sapiens
  source 1..1369: mol_type = other DNA; note = RFXANK
  CDS 506..1219: protein_id = 4; translation = MELTQPAEDLIQTQQTPASELGDPEDPGEEAADGSDTVVLSLFP CTPEPVNPEPDASVSSPQGSSLKHSTTLTNRQRGNEVSALPATLDCDNLVNKPDERGF TPLIWASAFGEIETVRFLLEWGADPHILAKERESALSLASTGGYTDIVGLLLERDVDI NIYDWNGGTPLLYAVRGNHVKCVEALLARGADLTTEADSGYTPMDLAVALGYRKVQQV IENHILKLFQSNLVPADPE
ccggacgccgcacggctcctgttccggtgtcagagggcccgccctccccgctcctcagtctttgcggacaagaaaggggctgtgtgagacgcagggaaggaggcacacccgggggtggcgcagtgaggagggggcgcgacggccaggaggctggtggagcgacacccaggcaggagagggggaagaactctctccctttctgaacccccttttccttgagagacgagttgggggagtcctccacgcattacccactcgggccgcaaaaactcccttctttagccctctgcccccgcccttgcttataagcctttgagaccgcagaagggaccttgttgtggaacgggacggccaagaggaagccagatcgctgagggtccggtctccagtttgcctcctgctatatccattggaagagaaaagtttgtgacttgggcccccaagttttgagagaactgggctttcggcgcggggggacagaggaggctcgtggggagctttccccatggagcttacccagcctgcagaagacctcatccagacccagcagacccctgcctcagaacttggggaccctgaagaccccggagaggaggctgcagatggctcagacactgtggtcctcagtctctttccctgcacccctgagcctgtgaatcctgaaccggatgccagtgtttcctctccacagggcagctccctgaagcactccaccactctcaccaaccggcagcgagggaacgaggtgtcagctctgccggccaccctagactgtgacaacctcgtcaacaagccagacgagcgcggcttcacccccctcatctgggcctccgcctttggagagattgagaccgttcgcttcctgctggagtggggtgccgacccccacatcctggcaaaagagcgagagagcgccctgtcgctggccagcacaggcggctacacagacattgtggggctgctgctggagcgtgacgtggacatcaacatctatgattggaatggagggacgccactgctgtacgctgtgcgcgggaaccacgtgaaatgcgttgaggccttgctggcccgaggcgctgacctcaccaccgaagccgactctggctacaccccgatggaccttgccgtggccctgggataccggaaagtgcaacaggtgatcgagaaccacatcctcaagctcttccagagcaacctggtgcccgctgaccctgagtgaaggccgcctgccggggactcagacactcagggaacaaaatggtcagccagagctggggaaacccagaactgacttcaaaggcagcttctggacaggtggtgggaggggacccttcccaagaggaaccaataaaccttctgtgcagaatga

SEQ ID NO: 4 - protein, 237 aa, Homo sapiens
  source 1..237: note = RFXANK
MELTQPAEDLIQTQQTPASELGDPEDPGEEAADGSDTVVLSLFPCTPEPVNPEPDASVSSPQGSSLKHSTTLTNRQRGNEVSALPATLDCDNLVNKPDERGFTPLIWASAFGEIETVRFLLEWGADPHILAKERESALSLASTGGYTDIVGLLLERDVDINIYDWNGGTPLLYAVRGNHVKCVEALLARGADLTTEADSGYTPMDLAVALGYRKVQQVIENHILKLFQSNLVPADPE

SEQ ID NO: 5 - DNA, 1438 bp, Homo sapiens
  source 1..1438: mol_type = other DNA; note = RFXANK
  CDS 506..1288: protein_id = 6; translation = MELTQPAEDLIQTQQTPASELGDPEDPGEEAADGSDTVVLSLFP CTPEPVNPEPDASVSSPQAGSSLKHSTTLTNRQRGNEVSALPATLDSLSIHQLAAQGE LDQLKEHLRKGDNLVNKPDERGFTPLIWASAFGEIETVRFLLEWGADPHILAKERESA LSLASTGGYTDIVGLLLERDVDINIYDWNGGTPLLYAVRGNHVKCVEALLARGADLTT EADSGYTPMDLAVALGYRKVQQVIENHILKLFQSNLVPADPE
ccggacgccgcacggctcctgttccggtgtcagagggcccgccctccccgctcctcagtctttgcggacaagaaaggggctgtgtgagacgcagggaaggaggcacacccgggggtggcgcagtgaggagggggcgcgacggccaggaggctggtggagcgacacccaggcaggagagggggaagaactctctccctttctgaacccccttttccttgagagacgagttgggggagtcctccacgcattacccactcgggccgcaaaaactcccttctttagccctctgcccccgcccttgcttataagcctttgagaccgcagaagggaccttgttgtggaacgggacggccaagaggaagccagatcgctgagggtccggtctccagtttgcctcctgctatatccattggaagagaaaagtttgtgacttgggcccccaagttttgagagaactgggctttcggcgcggggggacagaggaggctcgtggggagctttccccatggagcttacccagcctgcagaagacctcatccagacccagcagacccctgcctcagaacttggggaccctgaagaccccggagaggaggctgcagatggctcagacactgtggtcctcagtctctttccctgcacccctgagcctgtgaatcctgaaccggatgccagtgtttcctctccacaggcaggcagctccctgaagcactccaccactctcaccaaccggcagcgagggaacgaggtgtcagctctgccggccaccctagactccctgtccatccaccagctcgcagcacagggggagctggaccagctgaaggagcatttgcggaaaggtgacaacctcgtcaacaagccagacgagcgcggcttcacccccctcatctgggcctccgcctttggagagattgagaccgttcgcttcctgctggagtggggtgccgacccccacatcctggcaaaagagcgagagagcgccctgtcgctggccagcacaggcggctacacagacattgtggggctgctgctggagcgtgacgtggacatcaacatctatgattggaatggagggacgccactgctgtacgctgtgcgcgggaaccacgtgaaatgcgttgaggccttgctggcccgaggcgctgacctcaccaccgaagccgactctggctacaccccgatggaccttgccgtggccctgggataccggaaagtgcaacaggtgatcgagaaccacatcctcaagctcttccagagcaacctggtgcccgctgaccctgagtgaaggccgcctgccggggactcagacactcagggaacaaaatggtcagccagagctggggaaacccagaactgacttcaaaggcagcttctggacaggtggtgggaggggacccttcccaagaggaaccaataaaccttctgtgcagaatga

SEQ ID NO: 6 - protein, 260 aa, Homo sapiens
  source 1..260: note = RFXANK
MELTQPAEDLIQTQQTPASELGDPEDPGEEAADGSDTVVLSLFPCTPEPVNPEPDASVSSPQAGSSLKHSTTLTNRQRGNEVSALPATLDSLSIHQLAAQGELDQLKEHLRKGDNLVNKPDERGFTPLIWASAFGEIETVRFLLEWGADPHILAKERESALSLASTGGYTDIVGLLLERDVDINIYDWNGGTPLLYAVRGNHVKCVEALLARGADLTTEADSGYTPMDLAVALGYRKVQQVIENHILKLFQSNLVPADPE

SEQ ID NO: 7 - DNA, 3618 bp, Homo sapiens
  source 1..3618: mol_type = other DNA; note = RFX5
  CDS 222..2072: protein_id = 8; translation = MAEDEPDAKSPKTGGRAPPGGAEAGEPTTLLQRLRGTISKAVQN KVEGILQDVQKFSDNDKLYLYLQLPSGPTTGDKSSEPSTLSNEEYMYAYRWIRNHLEE HTDTCLPKQSVYDAYRKYCESLACCRPLSTANFGKIIREIFPDIKARRLGGRGQSKYC YSGIRRKTLVSMPPLPGLDLKGSESPEMGPEVTPAPRDELVEAACALTCDWAERILKR SFSSIVEVARFLLQQHLISARSAHAHVLKAMGLAEEDEHAPRERSSKPKNGLENPEGG AHKKPERLAQPPKDLEARTGAGPLARGERKKSVVESSAPGANNLQVNALVARLPLLLP RAPRSLIPPIPVSPPILAPRLSSGALKVATLPLSSRAGAPPAAVPIINMILPTVPALP GPGPGPGRAPPGGLTQPRGTENREVGIGGDQGPHDKGVKRTAEVPVSEASGQAPPAKA AKQDIEDTASDAKRKRGRPRKKSGGSGERNSTPLKSAAAMESAQSSRLPWETWGSGGE GNSAGGAERPGPMGEAEKGAVLAQGQGDGTVSKGGRGPGSQHTKEAEDKIPLVPSKVS VIKGSRSQKEAFPLAKGEVDTAPQGNKDLKEHVLQSSLSQEHKDPKATPP
attcagaaaaaggaaaataaagcaacgcacgagccggctgggccaggaccttggggctgggctggagtgaaggtggctacgagttttccagatttaggagacttcagaaaggtggggcagatagaatggagatggcaaagatctctttgggcatatatgggcctggcgaagtaatggaataatttctaattttcggagaaggcaagtgccctcatgccgggatggcagaagatgagcctgatgctaagagccccaagactgggggaagggcccccccaggtggtgctgaggctggggaacctaccacccttcttcagaggctccgaggtaccatttccaaggccgtgcagaacaaagtagaggggatcctgcaagatgtacagaaattttctgacaatgacaagctgtatctctaccttcagctcccctcaggacccaccactggagacaaaagctcagagccaagtacactgagcaatgaggagtacatgtatgcctataggtggatccgcaaccacctggaagagcacactgacacctgtctgccaaagcaaagtgtttatgatgcctatcggaagtactgtgagagtcttgcctgttgccgcccactcagcacagccaactttggcaagatcatcagagagatcttccctgacatcaaagctcgaaggcttggtggccggggccagtccaaatattgctacagtggcataaggaggaagaccttggtgtctatgccacccctgcctggacttgacctaaagggttctgagagtccagaaatgggcccagaagtaaccccagcacctcgagatgaactggtggaggcagcgtgtgccctgacctgtgactgggcagagcggatcctgaaacggtccttcagttccatcgttgaggtcgcccgcttcctgctacagcagcatctcatctctgcccgatctgcacatgcccatgtgcttaaggccatggggcttgctgaagaggacgaacatgcacctcgggaacggtcatctaaaccaaagaatggtttagagaacccagagggtggagcccacaagaagccagagagactggcccagcctcctaaggatctggaagcccgaactggggccggtcctctcgcacgtggagagcggaagaagagtgtagttgagagctcggccccaggagccaataacctgcaggttaatgccctagtggctcggctgcctctgctccttccccgggcccctcgctcactaattccgccaatcccagtctctccacctattctggcccccaggctttcttcaggtgccctgaaagtggctacactgcctctgtctagtagggccggggcacccccagcagctgtgcccatcattaacatgatcttaccaactgttcctgctttgcctggacctggacctgggcctgggcgagctccacctgggggactcactcagccccggggcacagagaacagagaggtaggcataggtggtgaccaaggaccacatgacaagggtgtcaagaggacagctgaagtacctgtgagtgaggccagtgggcaggctccaccagctaaagcagcaaagcaggatatagaggatacagcaagtgatgccaaaaggaaacgggggcgccctcgaaaaaagtcaggtggaagtggggaaaggaattctacccctctcaagtcagcagctgccatggaatctgcccagtcctcaaggttaccatgggagacatggggctcaggaggggaaggcaactcagctggaggggcagagaggccagggccaatgggagaggctgaaaagggggcagtacttgcccagggtcagggagatggtactgtttccaaaggaggaaggggccccggttcccagcataccaaagaagcagaagataaaattcccttggtcccctcaaaagtgagtgtcatcaagggcagcagaagccaaaaggaggcttttcctttggcaaagggagaggtagacactgcaccacagggtaataaagacttaaaggagcatgtgcttcaaagttccttatcccaggagcataaagacccaaaagcaacacccccatgatacaggtctgtggggaagagtgtttatatccctacgttaactttgcctagtagaggcccttctttgcacttgcttctcatttggctattcttttcctaaggaagtccattctcctctgtacagacagctgagtcacccagtctacttagtacctggttgctgcctctgaccttttcagcttgataccctgggctttagtgtaaccaataaatctgtagtgaccttacctgtattccctgtgctatcctgtgggaaggtaggaatgggctaagtatgatgaatatataggttagggatcttttggttttaaatcacagaaaacctaattcaaactggcttaaaataaaaaggatttattggttcatgtaactagaaagtccataggtagtgctggctccaggtgaagacttgacccagtagttcagtatgtctctaaataccggactgacttttttctcactgttgcatcttctgtaggaccatttaagtctgggccacttaatggctgccagcattcctaagattacacttttccccatttatgtccaatcagaaaaagaaggcatctttgtaccagaaatctcagcaaaagccctaatattcacactgattaggcctgggtcacatgtccaccctgaccaatcactgtggccaggaggatgatacatgctaatttgcttattctatatcatggacaacacctttggggaaaagggtgggggtcagcctccccaaaatcacatggattccccaagtggaaactaggagcagggagttgcttgggtggccgctaacaccaggctactcttattttagcttgctaagttgagatcagctagacctgctttcttttctcctcagtcttgcatttccctcaatacaagctgtagcctctttcctcgtttctagtctcagaaggaaggagagggaagccattctcctctagggactcttcagtctcatttagatgatagtccctttttttctacctccatattagagatggagctccttccttttcctgtttcttaatttttgtcttctcattcctgcttccctctcaccctattgccagttccaccaactagagtgaaagacttcctagccatttcattaaatctattctgtatccaccaggtggcagcatcttgtcatacgtgtcaggacttaggactgcggggtttaggttagatgtcacggaaaaagctagttctgtggtcaggcggcaccaatgagaaaggaatgcagaccctccagatgtatccttgggaaaagcagtaaaccaactaatatttattgaagacctactttgtcctctacatagggtagcttctgtcagggaatcttggttcttcccaagaaacactgattttctttcagggagacttcatgtgttcatttatttccaccacagcagattttaagaaattataatatgtaatatttgatatctataaagagtatatctaacgtgaataaattatgaagcatactaatgagtacctatgacccataacacatatacattaaaacattttaaatacca

SEQ ID NO: 8 - protein, 616 aa, Homo sapiens
  source 1..616: note = RFX5
MAEDEPDAKSPKTGGRAPPGGAEAGEPTTLLQRLRGTISKAVQNKVEGILQDVQKFSDNDKLYLYLQLPSGPTTGDKSSEPSTLSNEEYMYAYRWIRNHLEEHTDTCLPKQSVYDAYRKYCESLACCRPLSTANFGKIIREIFPDIKARRLGGRGQSKYCYSGIRRKTLVSMPPLPGLDLKGSESPEMGPEVTPAPRDELVEAACALTCDWAERILKRSFSSIVEVARFLLQQHLISARSAHAHVLKAMGLAEEDEHAPRERSSKPKNGLENPEGGAHKKPERLAQPPKDLEARTGAGPLARGERKKSVVESSAPGANNLQVNALVARLPLLLPRAPRSLIPPIPVSPPILAPRLSSGALKVATLPLSSRAGAPPAAVPIINMILPTVPALPGPGPGPGRAPPGGLTQPRGTENREVGIGGDQGPHDKGVKRTAEVPVSEASGQAPPAKAAKQDIEDTASDAKRKRGRPRKKSGGSGERNSTPLKSAAAMESAQSSRLPWETWGSGGEGNSAGGAERPGPMGEAEKGAVLAQGQGDGTVSKGGRGPGSQHTKEAEDKIPLVPSKVSVIKGSRSQKEAFPLAKGEVDTAPQGNKDLKEHVLQSSLSQEHKDPKATPP

SEQ ID NO: 9 - DNA, 3611 bp, Homo sapiens
  source 1..3611: mol_type = other DNA; note = RFX5
  CDS 215..2065: protein_id = 10; translation = MAEDEPDAKSPKTGGRAPPGGAEAGEPTTLLQRLRGTISKAVQN KVEGILQDVQKFSDNDKLYLYLQLPSGPTTGDKSSEPSTLSNEEYMYAYRWIRNHLEE HTDTCLPKQSVYDAYRKYCESLACCRPLSTANFGKIIREIFPDIKARRLGGRGQSKYC YSGIRRKTLVSMPPLPGLDLKGSESPEMGPEVTPAPRDELVEAACALTCDWAERILKR SFSSIVEVARFLLQQHLISARSAHAHVLKAMGLAEEDEHAPRERSSKPKNGLENPEGG AHKKPERLAQPPKDLEARTGAGPLARGERKKSVVESSAPGANNLQVNALVARLPLLLP RAPRSLIPPIPVSPPILAPRLSSGALKVATLPLSSRAGAPPAAVPIINMILPTVPALP GPGPGPGRAPPGGLTQPRGTENREVGIGGDQGPHDKGVKRTAEVPVSEASGQAPPAKA AKQDIEDTASDAKRKRGRPRKKSGGSGERNSTPLKSAAAMESAQSSRLPWETWGSGGE GNSAGGAERPGPMGEAEKGAVLAQGQGDGTVSKGGRGPGSQHTKEAEDKIPLVPSKVS VIKGSRSQKEAFPLAKGEVDTAPQGNKDLKEHVLQSSLSQEHKDPKATPP
attcagaaaaaggaaaataaagcaacgcacgagccggctgggccaggaccttggggctgggctggagtgaaggtggctacgagttttccagatttaggagacttcagaaaggtggggcagatagaatggagatggcaaagatctctttgggcatatatgggcctggcgaagtaatggaataatttctaattttcggagaagccctcatgccgggatggcagaagatgagcctgatgctaagagccccaagactgggggaagggcccccccaggtggtgctgaggctggggaacctaccacccttcttcagaggctccgaggtaccatttccaaggccgtgcagaacaaagtagaggggatcctgcaagatgtacagaaattttctgacaatgacaagctgtatctctaccttcagctcccctcaggacccaccactggagacaaaagctcagagccaagtacactgagcaatgaggagtacatgtatgcctataggtggatccgcaaccacctggaagagcacactgacacctgtctgccaaagcaaagtgtttatgatgcctatcggaagtactgtgagagtcttgcctgttgccgcccactcagcacagccaactttggcaagatcatcagagagatcttccctgacatcaaagctcgaaggcttggtggccggggccagtccaaatattgctacagtggcataaggaggaagaccttggtgtctatgccacccctgcctggacttgacctaaagggttctgagagtccagaaatgggcccagaagtaaccccagcacctcgagatgaactggtggaggcagcgtgtgccctgacctgtgactgggcagagcggatcctgaaacggtccttcagttccatcgttgaggtcgcccgcttcctgctacagcagcatctcatctctgcccgatctgcacatgcccatgtgcttaaggccatggggcttgctgaagaggacgaacatgcacctcgggaacggtcatctaaaccaaagaatggtttagagaacccagagggtggagcccacaagaagccagagagactggcccagcctcctaaggatctggaagcccgaactggggccggtcctctcgcacgtggagagcggaagaagagtgtagttgagagctcggccccaggagccaataacctgcaggttaatgccctagtggctcggctgcctctgctccttccccgggcccctcgctcactaattccgccaatcccagtctctccacctattctggcccccaggctttcttcaggtgccctgaaagtggctacactgcctctgtctagtagggccggggcacccccagcagctgtgcccatcattaacatgatcttaccaactgttcctgctttgcctggacctggacctgggcctgggcgagctccacctgggggactcactcagccccggggcacagagaacagagaggtaggcataggtggtgaccaaggaccacatgacaagggtgtcaagaggacagctgaagtacctgtgagtgaggccagtgggcaggctccaccagctaaagcagcaaagcaggatatagaggatacagcaagtgatgccaaaaggaaacgggggcgccctcgaaaaaagtcaggtggaagtggggaaaggaattctacccctctcaagtcagcagctgccatggaatctgcccagtcctcaaggttaccatgggagacatggggctcaggaggggaaggcaactcagctggaggggcagagaggccagggccaatgggagaggctgaaaagggggcagtacttgcccagggtcagggagatggtactgtttccaaaggaggaaggggccccggttcccagcataccaaagaagcagaagataaaattcccttggtcccctcaaaagtgagtgtcatcaagggcagcagaagccaaaaggaggcttttcctttggcaaagggagaggtagacactgcaccacagggtaataaagacttaaaggagcatgtgcttcaaagttccttatcccaggagcataaagacccaaaagcaacacccccatgatacaggtctgtggggaagagtgtttatatccctacgttaactttgcctagtagaggcccttctttgcacttgcttctcatttggctattcttttcctaaggaagtccattctcctctgtacagacagctgagtcacccagtctacttagtacctggttgctgcctctgaccttttcagcttgataccctgggctttagtgtaaccaataaatctgtagtgaccttacctgtattccctgtgctatcctgtgggaaggtaggaatgggctaagtatgatgaatatataggttagggatcttttggttttaaatcacagaaaacctaattcaaactggcttaaaataaaaaggatttattggttcatgtaactagaaagtccataggtagtgctggctccaggtgaagacttgacccagtagttcagtatgtctctaaataccggactgacttttttctcactgttgcatcttctgtaggaccatttaagtctgggccacttaatggctgccagcattcctaagattacacttttccccatttatgtccaatcagaaaaagaaggcatctttgtaccagaaatctcagcaaaagccctaatattcacactgattaggcctgggtcacatgtccaccctgaccaatcactgtggccaggaggatgatacatgctaatttgcttattctatatcatggacaacacctttggggaaaagggtgggggtcagcctccccaaaatcacatggattccccaagtggaaactaggagcagggagttgcttgggtggccgctaacaccaggctactcttattttagcttgctaagttgagatcagctagacctgctttcttttctcctcagtcttgcatttccctcaatacaagctgtagcctctttcctcgtttctagtctcagaaggaaggagagggaagccattctcctctagggactcttcagtctcatttagatgatagtccctttttttctacctccatattagagatggagctccttccttttcctgtttcttaatttttgtcttctcattcctgcttccctctcaccctattgccagttccaccaactagagtgaaagacttcctagccatttcattaaatctattctgtatccaccaggtggcagcatcttgtcatacgtgtcaggacttaggactgcggggtttaggttagatgtcacggaaaaagctagttctgtggtcaggcggcaccaatgagaaaggaatgcagaccctccagatgtatccttgggaaaagcagtaaaccaactaatatttattgaagacctactttgtcctctacatagggtagcttctgtcagggaatcttggttcttcccaagaaacactgattttctttcagggagacttcatgtgttcatttatttccaccacagcagattttaagaaattataatatgtaatatttgatatctataaagagtatatctaacgtgaataaattatgaagcatactaatgagtacctatgacccataacacatatacattaaaacattttaaatacca

SEQ ID NO: 10 - protein, 616 aa, Homo sapiens; identical to SEQ ID NO: 8
  source 1..616: note = RFX5

SEQ ID NO: 11 - DNA, 2825 bp, Homo sapiens
  source 1..2825: mol_type = other DNA; note = RFXAP
  CDS 157..975: protein_id = 12; translation = MEAQGVAEGAGPGAASGVPHPAALAPAAAPTLAPASVAAAASQF TLLVMQPCAGQDEAAAPGGSVGAGKPVRYLCEGAGDGEEEAGEDEADLLDTSDPPGGG ESAASLEDLEDEETHSGGEGSSGGARRRGSGGGSMSKTCTYEGCSETTSQVAKQRKPW MCKKHRNKMYKDKYKKKKSDQALNCGGTASTGSAGNVKLEESADNILSIVKQRTGSFG DRPARPTLLEQVLNQKRLSLLRSPEVVQFLQKQQQLLNQQVLEQRQQQFPGTSM
cgcgcaggctgcggtcgcgcaggcgcagtcggggcgccttcccggtataggcgccttttaccccagcgtgtcctgagtctttggttcgcgaagtgccgttaggccaagcaggtgctaaaagcccggggtcgtggaccccggccaggtcttagcagcatggaggcgcagggtgtagcggagggcgcggggccgggcgccgccagcggcgtgccccaccccgcggccctagccccggctgcggctcccaccttggcgccagcctcggtggcggccgcggcctctcaattcaccctgctagtgatgcaaccctgtgctgggcaggacgaggctgcggcccccgggggcagcgttggggcgggcaagcccgttaggtacctgtgcgaaggggccggggatggcgaagaggaggctggggaggacgaggcggacctgttagacacttcggaccctccggggggaggcgagagcgcggctagtttggaggatctagaggacgaggagactcactcggggggcgagggcagcagcgggggcgcccggaggcggggcagcggtgggggcagcatgagcaagacctgcacctacgaaggctgcagcgagaccacgagccaggtggccaagcagcgcaaaccgtggatgtgcaagaaacaccgcaacaagatgtacaaggacaagtataaaaagaagaagagcgaccaggccctgaactgcggtgggactgcctcgactggcagcgcgggaaacgtcaaactcgaggaaagtgcagataacatactctccattgttaaacaaagaacaggatcttttggggatcgtcctgcaagacctactcttttagaacaagtgttaaatcaaaaaagactgtcgttactaagaagtccagaagtagtgcaatttttacagaaacagcaacagctattaaatcagcaagttttggagcaaagacaacagcagtttccaggaacatcaatgtgagggaacttaccaagaacatctacatggttttttatcttattgtaatagatgagcatatttttttaccagacataaatggggtaataatctatgcctgtagaacataaacattttcctgtaaatgtatgtgtgcatttggggataagtaagtattgcactttgtgcatctaatctttcagattactgtgagtttgaagaagtcagcttatctttccaaataacatttaattataatgttttttaaaaaatatattcctcttcagtcattgttactgaaggtaatgaagcagttactttctgtggaagtcataaagttaatagatattaatcttgactcatctagctcagtggttctcatcaagggtcaatttgattgtcatagtgaccttgaaaaccactggcttttagtgagtggccaggaatgctaaatgttctgcagtgtcaggggtagtcccacatactaaagattgtctcacccgcagtgccaataacactcctaagaaatgttgatggctattttgtggtgctaacatgtagttggggcacctataattgggttctcttaataacctttctttgcagttaagactgaagctgtcaaagaggtaagcacattttatatagacgtaaggaaagtgattattgtttaatatctgtgaatttaggatgtgcatctcttttcagaggtgtgttagtaaaacctgacggattaactaagcacactgggatgtgtctcctacagttggcttctctctttgatgttacctgttagtgctgatctcttaaagcagacatttcttgtttgttgaatttgtgaacagtatagatctcagcccaccaatgccaagacaaaattatttttcttatacttattttttattaaacaaaatgaaaaagatccttttcaaaaaggtgatcctgaaaataaaactaacactccagtattttgtcattgtttttcgcaattgagctatctgaaaactgttattcctaagtaatgttcaaaaatgataagtaatctggatacctttttcttatactttctcctaggaaaactttaaaactttaaaaaggcaaacctaccaataggaataacaaattaaatgtcaagagagtatatccaatattaggatataaatgtatgtgtctcaagtttaactctacaaaaatttgttacttgttttttaaactctatatataaagttcgacttaatcatggctgttctaagaagtacttatggagagcaagaacgtttttgttcatttcttaatgtgtgtgtttttacttgcatatctgttcaaaacacttttaacaaaattaattcattaaagtccagttgttgacctttgagttagctgatttctttattctgttctttagtttattcttactagatgcagaggaattcatctactgtctgttattaactgttagtttattctcatacttacgatgttgagagtttttttgaagcttaagttaccctttatggtggaaaacattagcttatgcttctttagatggaataatgggaaaggagggaaatgggaaatggatggaaatgggaaaggagggaaaataatagcccagtgagagctgaatgaaaagggactgaatttaaatatttgtaagaactttgtgatgatgagtaattgtcagacgtgggatagataactgagaggctcagaatctttaccaaggatattttttaggataaggtagctgcctgttcatgaatttggataagaatagtaggacaatattcaacacaatttaatttttgtctgccacattagacatttttttaccttataaaatgatcaataaagcaataaggtttattttgggt

SEQ ID NO: 12 - protein, 272 aa, Homo sapiens
  source 1..272: note = RFXAP
MEAQGVAEGAGPGAASGVPHPAALAPAAAPTLAPASVAAAASQFTLLVMQPCAGQDEAAAPGGSVGAGKPVRYLCEGAGDGEEEAGEDEADLLDTSDPPGGGESAASLEDLEDEETHSGGEGSSGGARRRGSGGGSMSKTCTYEGCSETTSQVAKQRKPWMCKKHRNKMYKDKYKKKKSDQALNCGGTASTGSAGNVKLEESADNILSIVKQRTGSFGDRPARPTLLEQVLNQKRLSLLRSPEVVQFLQKQQQLLNQQVLEQRQQQFPGTSM

SEQ ID NO: 13 - DNA, 4654 bp, Homo sapiens
  source 1..4654: mol_type = other DNA; note = CIITA
  CDS 134..3526: protein_id = 14; translation = MRCLAPRPAGSYLSEPQGSSQCATMELGPLEGGYLELLNSDADP LCLYHFYDQMDLAGEEEIELYSEPDTDTINCDQFSRLLCDMEGDEETREAYANIAELD QYVFQDSQLEGLSKDIFKHIGPDEVIGESMEMPAEVGQKSQKRPFPEELPADLKHWKP AEPPTVVTGSLLVGPVSDCSTLPCLPLPALFNQEPASGQMRLEKTDQIPMPFSSSSLS CLNLPEGPIQFVPTISTLPHGLWQISEAGTGVSSIFIYHGEVPQASQVPPPSGFTVHG LPTSPDRPGSTSPFAPSATDLPSMPEPALTSRANMTEHKTSPTQCPAAGEVSNKLPKW PEPVEQFYRSLQDTYGAEPAGPDGILVEVDLVQARLERSSSKSLERELATPDWAERQL AQGGLAEVLLAAKEHRRPRETRVIAVLGKAGQGKSYWAGAVSRAWACGRLPQYDFVFS VPCHCLNRPGDAYGLQDLLFSLGPQPLVAADEVFSHILKRPDRVLLILDGFEELEAQD GFLHSTCGPAPAEPCSLRGLLAGLFQKKLLRGCTLLLTARPRGRLVQSLSKADALFEL SGFSMEQAQAYVMRYFESSGMTEHQDRALTLLRDRPLLLSHSHSPTLCRAVCQLSEAL LELGEDAKLPSTLTGLYVGLLGRAALDSPPGALAELAKLAWELGRRHQSTLQEDQFPS ADVRTWAMAKGLVQHPPRAAESELAFPSFLLQCFLGALWLALSGEIKDKELPQYLALT PRKKRPYDNWLEGVPRFLAGLIFQPPARCLGALLGPSAAASVDRKQKVLARYLKRLQP GTLRARQLLELLHCAHEAEEAGIWQHVVQELPGRLSFLGTRLTPPDAHVLGKALEAAG QDFSLDLRSTGICPSGLGSLVGLSCVTRFRAALSDTVALWESLQQHGETKLLQAAEEK FTIEPFKAKSLKDVEDLGKLVQTQRTRSSSEDTAGELPAVRDLKKLEFALGPVSGPQA FPKLVRILTAFSSLQHLDLDALSENKIGDEGVSQLSATFPQLKSLETLNLSQNNITDL GAYKLAEALPSLAASLLRLSLYNNCICDVGAESLARVLPDMVSLRVMDVQYNKFTAAG AQQLAASLRRCPHVETLAMWTPTIPFSVQEHLQQQDSRISLR
ggttagtgatgaggctagtgatgaggctgtgtgcttctgagctgggcatccgaaggcatccttggggaagctgagggcacgaggaggggctgccagactccgggagctgctgcctggctgggattcctacacaatgcgttgcctggctccacgccctgctgggtcctacctgtcagagccccaaggcagctcacagtgtgccaccatggagttggggcccctagaaggtggctacctggagcttcttaacagcgatgctgaccccctgtgcctctaccacttctatgaccagatggacctggctggagaagaagagattgagctctactcagaacccgacacagacaccatcaactgcgaccagttcagcaggctgttgtgtgacatggaaggtgatgaagagaccagggaggcttatgccaatatcgcggaactggaccagtatgtcttccaggactcccagctggagggcctgagcaaggacattttcaagcacataggaccagatgaagtgatcggtgagagtatggagatgccagcagaagttgggcagaaaagtcagaaaagacccttcccagaggagcttccggcagacctgaagcactggaagccagctgagccccccactgtggtgactggcagtctcctagtgggaccagtgagcgactgctccaccctgccctgcctgccactgcctgcgctgttcaaccaggagccagcctccggccagatgcgcctggagaaaaccgaccagattcccatgcctttctccagttcctcgttgagctgcctgaatctccctgagggacccatccagtttgtccccaccatctccactctgccccatgggctctggcaaatctctgaggctggaacaggggtctccagtatattcatctaccatggtgaggtgccccaggccagccaagtaccccctcccagtggattcactgtccacggcctcccaacatctccagaccggccaggctccaccagccccttcgctccatcagccactgacctgcccagcatgcctgaacctgccctgacctcccgagcaaacatgacagagcacaagacgtcccccacccaatgcccggcagctggagaggtctccaacaagcttccaaaatggcctgagccggtggagcagttctaccgctcactgcaggacacgtatggtgccgagcccgcaggcccggatggcatcctagtggaggtggatctggtgcaggccaggctggagaggagcagcagcaagagcctggagcgggaactggccaccccggactgggcagaacggcagctggcccaaggaggcctggctgaggtgctgttggctgccaaggagcaccggcggccgcgtgagacacgagtgattgctgtgctgggcaaagctggtcagggcaagagctattgggctggggcagtgagccgggcctgggcttgtggccggcttccccagtacgactttgtcttctctgtcccctgccattgcttgaaccgtccgggggatgcctatggcctgcaggatctgctcttctccctgggcccacagccactcgtggcggccgatgaggttttcagccacatcttgaagagacctgaccgcgttctgctcatcctagacggcttcgaggagctggaagcgcaagatggcttcctgcacagcacgtgcggaccggcaccggcggagccctgctccctccgggggctgctggccggccttttccagaagaagctgctccgaggttgcaccctcctcctcacagcccggccccggggccgcctggtccagagcctgagcaaggccgacgccctatttgagctgtccggcttctccatggagcaggcccaggcatacgtgatgcgctactttgagagctcagggatgacagagcaccaagacagagccctgacgctcctccgggaccggccacttcttctcagtcacagccacagccctactttgtgccgggcagtgtgccagctctcagaggccctgctggagcttggggaggacgccaagctgccctccacgctcacgggactctatgtcggcctgctgggccgtgcagccctcgacagcccccccggggccctggcagagctggccaagctggcctgggagctgggccgcagacatcaaagtaccctacaggaggaccagttcccatccgcagacgtgaggacctgggcgatggccaaaggcttagtccaacacccaccgcgggccgcagagtccgagctggccttccccagcttcctcctgcaatgcttcctgggggccctgtggctggctctgagtggcgaaatcaaggacaaggagctcccgcagtacctagcattgaccccaaggaagaagaggccctatgacaactggctggagggcgtgccacgctttctggctgggctgatcttccagcctcccgcccgctgcctgggagccctactcgggccatcggcggctgcctcggtggacaggaagcagaaggtgcttgcgaggtacctgaagcggctgcagccggggacactgcgggcgcggcagctgctggagctgctgcactgcgcccacgaggccgaggaggctggaatttggcagcacgtggtacaggagctccccggccgcctctcttttctgggcacccgcctcacgcctcctgatgcacatgtactgggcaaggccttggaggcggcgggccaagacttctccctggacctccgcagcactggcatttgcccctctggattggggagcctcgtgggactcagctgtgtcacccgtttcagggctgccttgagcgacacggtggcgctgtgggagtccctgcagcagcatggggagaccaagctacttcaggcagcagaggagaagttcaccatcgagcctttcaaagccaagtccctgaaggatgtggaagacctgggaaagcttgtgcagactcagaggacgagaagttcctcggaagacacagctggggagctccctgctgttcgggacctaaagaaactggagtttgcgctgggccctgtctcaggcccccaggctttccccaaactggtgcggatcctcacggccttttcctccctgcagcatctggacctggatgcgctgagtgagaacaagatcggggacgagggtgtctcgcagctctcagccaccttcccccagctgaagtccttggaaaccctcaatctgtcccagaacaacatcactgacctgggtgcctacaaactcgccgaggccctgccttcgctcgctgcatccctgctcaggctaagcttgtacaataactgcatctgcgacgtgggagccgagagcttggctcgtgtgcttccggacatggtgtccctccgggtgatggacgtccagtacaacaagttcacggctgccggggcccagcagctcgctgccagccttcggaggtgtcctcatgtggagacgctggcgatgtggacgcccaccatcccattcagtgtccaggaacacctgcaacaacaggattcacggatcagcctgagatgatcccagctgtgctctggacaggcatgttctctgaggacactaaccacgctggaccttgaactgggtacttgtggacacagctcttctccaggctgtatcccatgagcctcagcatcctggcacccggcccctgctggttcagggttggcccctgcccggctgcggaatgaaccacatcttgctctgctgacagacacaggcccggctccaggctcctttagcgcccagttgggtggatgcctggtggcagctgcggtccacccaggagccccgaggccttctctgaaggacattgcggacagccacggccaggccagagggagtgacagaggcagccccattctgcctgcccaggcccctgccaccctggggagaaagtacttctttttttttatttttagacagagtctcactgttgcccaggctggcgtgcagtggtgcgatctgggttcactgcaacctccgcctcttgggttcaagcgattcttctgcttcagcctcccgagtagctgggactacaggcacccaccatcatgtctggctaatttttcatttttagtagagacagggttttgccatgttggccaggctggtctcaaactcttgacctcaggtgatccacccacctcagcctcccaaagtgctgggattacaagcgtgagccactgcaccgggccacagagaaagtacttctccaccctgctctccgaccagacaccttgacagggcacaccgggcactcagaagacactgatgggcaacccccagcctgctaattccccagattgcaacaggctgggcttcagtggcagctgcttttgtctatgggactcaatgcactgacattgttggccaaagccaaagctaggcctggccagatgcaccagcccttagcagggaaacagctaatgggacactaatggggcggtgagaggggaacagactggaagcacagcttcatttcctgtgtcttttttcactacattataaatgtctctttaatgtcacaggcaggtccagggtttgagttcataccctgttaccattttggggtacccactgctctggttatctaatatgtaacaagccaccccaaatcatagtggcttaaaacaacactcacattta

SEQ ID NO: 14 - protein, 1130 aa, Homo sapiens
  source 1..1130: note = CIITA
MRCLAPRPAGSYLSEPQGSSQCATMELGPLEGGYLELLNSDADPLCLYHFYDQMDLAGEEEIELYSEPDTDTINCDQFSRLLCDMEGDEETREAYANIAELDQYVFQDSQLEGLSKDIFKHIGPDEVIGESMEMPAEVGQKSQKRPFPEELPADLKHWKPAEPPTVVTGSLLVGPVSDCSTLPCLPLPALFNQEPASGQMRLEKTDQIPMPFSSSSLSCLNLPEGPIQFVPTISTLPHGLWQISEAGTGVSSIFIYHGEVPQASQVPPPSGFTVHGLPTSPDRPGSTSPFAPSATDLPSMPEPALTSRANMTEHKTSPTQCPAAGEVSNKLPKWPEPVEQFYRSLQDTYGAEPAGPDGILVEVDLVQARLERSSSKSLERELATPDWAERQLAQGGLAEVLLAAKEHRRPRETRVIAVLGKAGQGKSYWAGAVSRAWACGRLPQYDFVFSVPCHCLNRPGDAYGLQDLLFSLGPQPLVAADEVFSHILKRPDRVLLILDGFEELEAQDGFLHSTCGPAPAEPCSLRGLLAGLFQKKLLRGCTLLLTARPRGRLVQSLSKADALFELSGFSMEQAQAYVMRYFESSGMTEHQDRALTLLRDRPLLLSHSHSPTLCRAVCQLSEALLELGEDAKLPSTLTGLYVGLLGRAALDSPPGALAELAKLAWELGRRHQSTLQEDQFPSADVRTWAMAKGLVQHPPRAAESELAFPSFLLQCFLGALWLALSGEIKDKELPQYLALTPRKKRPYDNWLEGVPRFLAGLIFQPPARCLGALLGPSAAASVDRKQKVLARYLKRLQPGTLRARQLLELLHCAHEAEEAGIWQHVVQELPGRLSFLGTRLTPPDAHVLGKALEAAGQDFSLDLRSTGICPSGLGSLVGLSCVTRFRAALSDTVALWESLQQHGETKLLQAAEEKFTIEPFKAKSLKDVEDLGKLVQTQRTRSSSEDTAGELPAVRDLKKLEFALGPVSGPQAFPKLVRILTAFSSLQHLDLDALSENKIGDEGVSQLSATFPQLKSLETLNLSQNNITDLGAYKLAEALPSLAASLLRLSLYNNCICDVGAESLARVLPDMVSLRVMDVQYNKFTAAGAQQLAASLRRCPHVETLAMWTPTIPFSVQEHLQQQDSRISLR

SEQ ID NO: 15 - DNA, 2679 bp, Homo sapiens
  source 1..2679: mol_type = other DNA; note = HLA-E
  CDS 127..1203: protein_id = 16; translation = MVDGTLLLLLSEALALTQTWAGSHSLKYFHTSVSRPGRGEPRFI SVGYVDDTQFVRFDNDAASPRMVPRAPWMEQEGSEYWDRETRSARDTAQIFRVNLRTL RGYYNQSEAGSHTLQWMHGCELGPDGRFLRGYEQFAYDGKDYLTLNEDLRSWTAVDTA AQISEQKSNDASEAEHQRAYLEDTCVEWLHKYLEKGKETLLHLEPPKTHVTHHPISDH EATLRCWALGFYPAEITLTWQQDGEGHTQDTELVETRPAGDGTFQKWAAVVVPSGEEQ RYTCHVQHEGLPEPVTLRWKPASQPTIPIVGIIAGLVLLGSVVSGAVVAAVIWRKKSS GGKGGSYSKAEWSDSAQGSESHSL
gcagactcagttctcattcccaatgggtgtcgggtttctagagaagccaatcagcgtcgccacgactcccgactataaagtccccatccggactcaagaagttctcaggactcagaggctgggatcatggtagatggaaccctccttttactcctctcggaggccctggcccttacccagacctgggcgggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaaagcctgagacagctgccttgtgtgcgactgagatgcacagctgccttgtgtgcgactgagatgcaggatttcctcacgcctcccctatgtgtcttaggggactctggcttctctttttgcaagggcctctgaatctgtctgtgtccctgttagcacaatgtgaggaggtagagaaacagtccacctctgtgtctaccatgacccccttcctcacactgacctgtgttccttccctgttctcttttctattaaaaataagaacctgggcagagtgcggcagctcatgcctgtaatcccagcacttagggaggccgaggagggcagatcacgaggtcaggagatcgaaaccatcctggctaacacggtgaaaccccgtctctactaaaaaatacaaaaaattagctgggcgcagaggcacgggcctgtagtcccagctactcaggaggcggaggcaggagaatggcgtcaacccgggaggcggaggttgcagtgagccaggattgtgcgactgcactccagcctgggtgacagggtgaaacgccatctcaaaaaataaaaattgaaaaataaaaaaagaacctggatctcaatttaatttttcatattcttgcaatgaaatggacttgaggaagctaagatcatagctagaaatacagataattccacagcacatctctagcaaatttagcctattcctattctctagcctattccttaccacctgtaatcttgaccatataccttggagttgaatattgttttcatactgctgtggtttgaatgttccctccaacactcatgttgagacttaatccctaatgtggcaatactgaaaggtggggcctttgagatgtgattggatcgtaaggctgtgccttcattcatgggttaatggattaatgggttatcacaggaatgggactggtggctttataagaagaggaaaagagaactgagctagcatgcccagcccacagagagcctccactagagtgatgctaagtggaaatgtgaggtgcagctgccacagagggcccccaccagggaaatgtctagtgtctagtggatccaggccacaggagagagtgccttgtggagcgctgggagcaggacctgaccaccaccaggaccccagaactgtggagtcagtggcagcatgcagcgcccccttgggaaagctttaggcaccagcctgcaacccattcgagcagccacgtaggctgcacccagcaaagccacaggcacggggctacctgaggccttgggggcccaatccctgctccagtgtgtccgtgaggcagcacacgaagtcaaaagagattattctcttcccacagataccttttctctcccatgaccctttaacagcatctgcttcattcccctcaccttcccaggctgatctgaggtaaactttgaagtaaaataaaagctgtgtttgagcatcatttgtatttcaaaaaaaaaaaaaaaaaa

SEQ ID NO: 16 - protein, 358 aa, Homo sapiens
  source 1..358: note = HLA-E
MVDGTLLLLLSEALALTQTWAGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 17 - DNA, 1301 bp, Homo sapiens
  source 1..1301: mol_type = other DNA; note = HLA-F
  CDS 125..1165: protein_id = 18; translation = MAPRSLLLLLSGALALTDTWAGSHSLRYFSTAVSRPGRGEPRYI AVEYVDDTQFLRFDSDAAIPRMEPREPWVEQEGPQYWEWTTGYAKANAQTDRVALRNL LRRYNQSEAGSHTLQGMNGCDMGPDGRLLRGYHQHAYDGKDYISLNEDLRSWTAADTV AQITQRFYEAEEYAEEFRTYLEGECLELLRRYLENGKETLQRADPPKAHVAHHPISDH EATLRCWALGFYPAEITLTWQRDGEEQTQDTELVETRPAGDGTFQKWAAVVVPPGEEQ RYTCHVQHEGLPQPLILRWEQSPQPTIPIVGIVAGLVVLGAVVTGAVVAAVMWRKKSS DRNRGSYSQAAV
tttctcactcccattgggcgtcgcgtttctagagaagccaatcagtgtcgccgcagttcccaggttctaaagtcccacgcaccccgcgggactcatatttttcccagacgcggaggttggggtcatggcgccccgaagcctcctcctgctgctctcaggggccctggccctgaccgatacttgggcgggctcccactccttgaggtatttcagcaccgctgtgtcgcggcccggccgcggggagccccgctacatcgccgtggagtacgtagacgacacgcaattcctgcggttcgacagcgacgccgcgattccgaggatggagccgcgggagccgtgggtggagcaagaggggccgcagtattgggagtggaccacagggtacgccaaggccaacgcacagactgaccgagtggccctgaggaacctgctccgccgctacaaccagagcgaggctgggtctcacaccctccagggaatgaatggctgcgacatggggcccgacggacgcctcctccgcgggtatcaccagcacgcgtacgacggcaaggattacatctccctgaacgaggacctgcgctcctggaccgcggcggacaccgtggctcagatcacccagcgcttctatgaggcagaggaatatgcagaggagttcaggacctacctggagggcgagtgcctggagttgctccgcagatacttggagaatgggaaggagacgctacagcgcgcagatcctccaaaggcacacgttgcccaccaccccatctctgaccatgaggccaccctgaggtgctgggccctgggcttctaccctgcggagatcacgctgacctggcagcgggatggggaggaacagacccaggacacagagcttgtggagaccaggcctgcaggggatggaaccttccagaagtgggccgctgtggtggtgcctcctggagaggaacagagatacacatgccatgtgcagcacgaggggctgccccagcccctcatcctgagatgggagcagtctccccagcccaccatccccatcgtgggcatcgttgctggccttgttgtccttggagctgtggtcactggagctgtggtcgctgctgtgatgtggaggaagaagagctcagatagaaacagagggagctactctcaggctgcagtgtgagacagcttccttgtgtgggactgagaagcaagatatcaatgtagcagaattgcacttgtgcctcacgaacatacataaattttaaaaataaagaataaaaatatatctttttatagataaaaaaaaaaaaaaaaaa

SEQ ID NO: 18 - protein, 346 aa, Homo sapiens
  source 1..346: note = HLA-F
MAPRSLLLLLSGALALTDTWAGSHSLRYFSTAVSRPGRGEPRYIAVEYVDDTQFLRFDSDAAIPRMEPREPWVEQEGPQYWEWTTGYAKANAQTDRVALRNLLRRYNQSEAGSHTLQGMNGCDMGPDGRLLRGYHQHAYDGKDYISLNEDLRSWTAADTVAQITQRFYEAEEYAEEFRTYLEGECLELLRRYLENGKETLQRADPPKAHVAHHPISDHEATLRCWALGFYPAEITLTWQRDGEEQTQDTELVETRPAGDGTFQKWAAVVVPPGEEQRYTCHVQHEGLPQPLILRWEQSPQPTIPIVGIVAGLVVLGAVVTGAVVAAVMWRKKSSDRNRGSYSQAAV

SEQ ID NO: 19 - DNA, 1578 bp, Homo sapiens
  source 1..1578: mol_type = other DNA; note = HLA-G
  CDS 179..1195: protein_id = 20; translation = MVVMAPRTLFLLLSGALTLTETWAGSHSMRYFSAAVSRPGRGEP RFIAMGYVDDTQFVRFDSDSACPRMEPRAPWVEQEGPEYWEEETRNTKAHAQTDRMNL QTLRGYYNQSEASSHTLQWMIGCDLGSDGRLLRGYEQYAYDGKDYLALNEDLRSWTAA DTAAQISKRKCEAANVAEQRRAYLEGTCVEWLHRYLENGKEMLQRADPPKTHVTHHPV FDYEATLRCWALGFYPAEIILTWQRDGEDQTQDVELVETRPAGDGTFQKWAAVVVPSG EEQRYTCHVQHEGLPEPLMLRWKQSSLPTIPIMGIVAGLVVLAAVVTGAAVAAVLWRK KSSD
agtgtggtactttgtcttgaggagatgtcctggactcacacggaaacttagggctacggaatgaagttctcactcccattaggtgacaggtttttagagaagccaatcagcgtcgccgcggtcctggttctaaagtcctcgctcacccacccggactcattctccccagacgccaaggatggtggtcatggcgccccgaaccctcttcctgctgctctcgggggccctgaccctgaccgagacctgggcgggctcccactccatgaggtatttcagcgccgccgtgtcccggcccggccgcggggagccccgcttcatcgccatgggctacgtggacgacacgcagttcgtgcggttcgacagcgactcggcgtgtccgaggatggagccgcgggcgccgtgggtggagcaggaggggccggagtattgggaagaggagacacggaacaccaaggcccacgcacagactgacagaatgaacctgcagaccctgcgcggctactacaaccagagcgaggccagttctcacaccctccagtggatgattggctgcgacctggggtccgacggacgcctcctccgcgggtatgaacagtatgcctacgatggcaaggattacctcgccctgaacgaggacctgcgctcctggaccgcagcggacactgcggctcagatctccaagcgcaagtgtgaggcggccaatgtggctgaacaaaggagagcctacctggagggcacgtgcgtggagtggctccacagatacctggagaacgggaaggagatgctgcagcgcgcggacccccccaagacacacgtgacccaccaccctgtctttgactatgaggccaccctgaggtgctgggccctgggcttctaccctgcggagatcatactgacctggcagcgggatggggaggaccagacccaggacgtggagctcgtggagaccaggcctgcaggggatggaaccttccagaagtgggcagctgtggtggtgccttctggagaggagcagagatacacgtgccatgtgcagcatgaggggctgccggagcccctcatgctgagatggaagcagtcttccctgcccaccatccccatcatgggtatcgttgctggcctggttgtccttgcagctgtagtcactggagctgcggtcgctgctgtgctgtggagaaagaagagctcagattgaaaaggagggagctactctcaggctgcaatgtgaaacagctgccctgtgtgggactgagtggcaagtccctttgtgacttcaagaaccctgactcctctttgtgcagagaccagcccacccctgtgcccaccatgaccctcttcctcatgctgaactgcattccttccccaatcacctttcctgttccagaaaaggggctgggatgtctccgtctctgtctcaaatttgtggtccactgagctataacttacttctgtattaaaattagaatctgagtataaatttactttttcaaattatttccaagagagattgatgggttaattaaaggagaagattcctgaaatttgagagacaaaataaatggaagacatgagaacttt

SEQ ID NO: 20 - protein, 338 aa, Homo sapiens
  source 1..338: note = HLA-G
MVVMAPRTLFLLLSGALTLTETWAGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD